(19) United States
(12) Patent Application Publication
(10) Pub. No.: US 2005/0119489 A1
(43) Pub. Date: Jun. 2, 2005

(54) 4-Sulfide/sulfoxide/sulfonyl-1h-pyrazolyl derivative compounds, for use in diseases associated with the 5-ht2c receptor

(76) Inventors: Gaetan H. Ladouceur, Guilford, CT (US); Emil Velthuisen, Irvine, CA (US); Soongyu Choi, Skillman, NJ (US); Zhonghua Zhang, Derby, CT (US); Yamin Wang, Sandy Hook, CT (US); Jeremy L. Baryza, Mountain View, CA (US); Philip Coish, New Haven, CT (US); Roger Smith, Madison, CT (US); Jinshan Chen, Clinton, CT (US)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/499,789

(22) PCT Filed: Dec. 28, 2002
(86) PCT No.: PCT/US02/41635

Related U.S. Application Data
(60) Provisional application No. 60/343,749, filed on Dec. 28, 2001.

(51) Int. Cl. C07D 231/18; A61K 31/4152

(52) U.S. Cl. 548/370.1; 514/407

(57) ABSTRACT

Disclosed are 4-sulfide/sulfoxide/sulfonyl-1H-pyrazolyl derivative compounds of formulas (I) or (II): wherein the variables n, R, R₁, R₂, R₃, R_{3′}, R₄, R_{4′}, R₅ and R₆ are as defined in the specification. The compounds are useful for the treatment or prevention of diseases and/or behaviors involving the 5-HT_{2C} receptor.

The present invention relates to:
(1) 4-sulfide/sulfoxide/sulfonyl-1H-pyrazolyl derivative compounds or purified stereoisomers or stereoisomer mixtures of said compounds and salts or prodrug forms thereof;
(2) Pharmaceutical compositions comprising one or more of the compounds or purified stereoisomers or stereoisomer mixtures of the invention, or their salt or prodrug forms thereof, with a pharmaceutically acceptable ingredient;
(3) Methods of preparing the compounds of (1); and
(4) Methods of treating diseases associated with the 5-HT_{2C} receptor in mammals by administering an effective amount of (1) or (2) to a patient in need thereof.
   Description of the Compounds and Intermediates Thereof

The 4-sulfide/sulfoxide/sulfonyl-1H-pyrazolyl derivative compounds or purified stereoisomers or stereoisomer mixtures of said compounds and their salts or prodrug forms thereof have structural formulae: 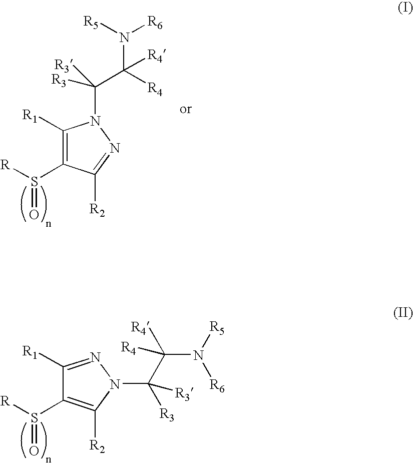
wherein:
n is 0, 1 or 2;
R is selected from the group consisting of:
   (a) (C₁-C₆)-alkyl optionally substituted by a substituent selected from the group consisting of:
      (b1) halogen,
      (b2) cyano,
      (b3) (C₁-C₅)-alkoxy,
      (b4) (C₆-C₁₀)-aryloxy,
      (b5) C(═O)NR₇R₈,
      (b6) (C₃-C₈)-cycloalkyl, and
      (b7) (C₆-C₁₀)-aryl optionally substituted with one to three substituents selected from the group consisting of cyano, halogen, nitro, (C₁-C₅)-alkyl, (C₁-C₅)-alkoxy, phenyl and arylsulfonyl,
   (b) (C₁-C₅)-alkenyl optionally substituted with (C₁-C₅)-alkyl,
   (c) (C₁-C₅)-alkynyl optionally substituted with (C₁-C₅)-alkyl,
   (d) (C₆-C₁₀)-aryl which is optionally substituted with one to three substituents selected from the group consisting of:
      (d1) halogen,
      (d2) nitro,
      (d3) (C₁-C₅)-alkyl optionally substituted with halogen,
      (d4) (C₁-C₅)-alkenyl optionally substituted with (C₁-C₅)-alkyl,
      (d5) (C₁-C₅)-alkynyl optionally substituted with (C₁-C₅)-alkyl,
      (d6) (C₁-C₅)-alkoxy,
      (d7) NR₉C(═O)R₁₀,
      (d8) NR₉S(═O)ₙ—R₁₀,
      (d9) NR₉C(═S)R₁₀,
      (d10) NR₁₁R₁₂,
      (d11) C(═O)R₁₀,
      (d12) C(═O)NR₁₃R₁₄,
      (d13) C(═O)OR₁₅,
      (d14) (C₆-C₁₀)-aryl optionally substituted with one to three substituents selected from the group consisting of:
         (d14a) halogen,
         (d14b) (C₁-C₅)-alkyl,
         (d14c) (C₁-C₅)-alkoxy,
         (d14d) a four to eight membered saturated or unsaturated heterocyclic ring which contains one to four heteroatoms selected from the group consisting of nitrogen, oxygen and sulfur, wherein said heterocyclic ring contains at least one carbon atom and wherein said heterocyclic ring is optionally substituted with one to four substituents selected from the group consisting of:
            (d14d1) nitro,
            (d14d2) NR₉C(═O)R₁₀,
            (d14d3) oxo,
            (d14d4) (C₁-C₅) alkyl optionally substituted with halogen,
            (d14d5) C(═O)R₁₅,
            (d14d6) C(═O)OR₁₅,
            (d14d7) C(═O)NR₁₃R₁₄,
            (d14d8) (C₆-C₁₀)-aryl optionally substituted with halogen, and
            (d14d9) (C₃-C₈)-cycloalkyl ring, and
         (d14e) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atom and said heterocyclic ring is optionally substituted with one to two oxo substituents, and the other ring is a saturated or unsaturated three to eight membered cycloalkyl ring,
      (d15) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atom, and the other ring is a saturated or unsaturated three to eight membered cycloalkyl ring;
      (d16) C(═O)OR₁₅,
      (d17) OH, and
      (d18) CN;
   (e) a four to eight membered saturated or unsaturated heterocyclic ring which contains one to four heteroatoms selected from the group consisting of nitrogen, oxygen and sulfur wherein said heterocyclic ring contains at least one carbon atom wherein said heterocyclic ring is optionally substituted with one to four substituents selected from the group consisting of:
      (e1) nitro,
      (e2) NR₉C(═O)R₁₀,
      (e3) oxo,
      (e4) (C₁-C₅)-alkyl optionally substituted with halogen,
      (e5) C(═O)R₁₅,
      (e6) C(═O)OR₁₅,
      (e7) C(═O)NR₁₃R₁₄,
      (e8) (C₆-C₁₀)-aryl optionally substituted with halogen, and
      (e9) (C₃-C₈)-cycloalkyl ring, and
   (f) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atoms, and the other ring is a saturated or unsaturated three to eight membered cycloalkyl ring;
R₁ and R₂ are independently selected from the group consisting of.
   (a) hydrogen,
   (b) hydroxy,
   (c) (C₁-C₅)-alkyl optionally substituted with halogen or hydroxy,
   (d) (C₁-C₅)-alkoxy,
   (e) (C₁-C₅)-alkoxy-(C₁-C₅)-alkyl,
   (f) (C₆-C₁₀)-aryl-(C₁-C₅)-alkoxy- wherein the (C₆-C₁₀)-aryl is optionally substituted with halogen,
   (g) C(═O)R₁₅, and
   (h) C(═O)NR₁₇R₁₈;
R₃, R_{3′}, R₄ and R_{4′} are independently selected from the group consisting of:
   (a) hydrogen,
   (b) (C₁-C₅)-all,
   (c) (C₆-C₁₀)-aryl,
   (d) (C₆-C₁₀)-aryl-(C₁-C₅)-alkyl, and
   (e) (C₃-C₈)-cycloalkyl ring,
R₃ and R₄ together form a four to eight membered saturated or unsaturated carbocyclic ring, or
R₄ and R_{4′} together form a C₃-C₈-cycloalkyl ring;
R₅ and R₆ are independently selected from the group consisting of hydrogen and C₁-C₅-alkyl, or
the carbon to which R₄ and R_{4′} are attached and NR₅R₆ form a —CN wherein R₄ and R₅ form a bond and R_{4′} and R₆ form a bond, or
R₃, R₄ and NR₅R₆ together form a four to eight membered saturated or unsaturated heterocyclic ring wherein the nitrogen represents the only heteroatom;
R₇ and R₈ are independently selected from the group consisting of:
   (a) hydrogen,
   (b) (C₁-C₆)-alkyl optionally substituted with (C₁-C₅)-alkoxy or a four to eight membered heterocyclic ring which contains one to four heteroatoms selected from the group consisting of nitrogen, oxygen and sulfur wherein said heterocyclic ring contains at least one carbon atom,
   (c) (C₆-C₁₀)-aryl optionally substituted with one to three substituents selected from the group consisting of halogen, (C₁-C₅)-alkoxy or (C₁-C₅)-alkyl optionally substituted by halogen,
   (d) (C₆-C₁₀)-aryl-(C₁-C₅)-alkyl wherein the (C₆-C₁₀)-aryl is optionally substituted with one to three substituents selected from the group consisting of halogen, (C₁-C₅)-alkoxy or (C₁-C₅)-alkyl optionally substituted by halogen, and
   (e) (C₃-C₈)-cycloalkyl is optionally substituted with one to three substituents selected from the group consisting of halogen, (C₁-C₅)-alkyl, or (C₁-C₅)-alkoxy,
R₉ is hydrogen or (C₁-C₅)-alkyl;
R₁₀ is selected from the group consisting of:
   (a) (C₁-C₅)-alkyl optionally substituted with C₃-C₈-carbocyclic ring or C₆-C₁₀-aryl optionally substituted with halogen,
   (b) (C₁-C₅)-alkoxy,
   (c) (C₃-C₈)-cycloalkyl optionally substituted with one to three substituents selected from the group consisting of halogen, (C₁-C₅)-alkoxy or (C₁-C₅)-alkyl optionally substituted by halogen,
   (d) a bicyclo cycloalkyl ring wherein each ring is independently a five to six membered cycloalkyl ring,
   (e) a tricyclo cycloalkyl ring wherein each ring is independently a five to six membered cycloalkyl ring,
   (f) (C₆-C₁₀)-aryl optionally substituted with one to three substituents selected from the group consisting of halogen, (C₁-C₅)-alkoxy and (C₁-C₅)-alkyl optionally substituted by halogen,
   (g) —NR₁₁R₁₂, and
   (h) a four to eight membered saturated or unsaturated heterocyclic ring which contains one to four heteroatoms selected from the group consisting of nitrogen, oxygen and sulfur wherein said heterocyclic ring contains at least two carbon atoms wherein said heterocyclic ring is optionally substituted with one to four substituents selected from the group consisting of
      (h1) nitro,
      (h2) NR₉C(═O)R₁₀,
      (h3) oxo,
      (h4) (C₁-C₅)-alkyl optionally substituted with halogen,
      (h5) C(═O)R₁₅,
      (h6) C(═O)OR₁₅,
      (h7) C(═O)NR₁₃R₁₄,
      (h8) (C₆-C₁₀)-aryl optionally substituted with halogen, and
      (h9) (C₃-C₈)-cycloalkyl ring;
R₁₁, R₁₂, R₁₃, R₁₄, R₁₇ and R₁₈ are independently selected from the group consisting of:
   (a) hydrogen,
   (b) (C₁-C₅)-alkyl,
   (c) (C₃-C₈)-cycloalkyl,
   (d) (C₆-C₁₀)-aryl, and
   (e) (C₆-C₁₀)-aryl-(C₁-C₅)-alkyl;
R¹⁵ is hydrogen or (C₁-C₅)-alkyl;
   or a purified stereoisomer or stereoisomer mixture of said compound, or salt of said compound, stereoisomer or stereoisomer mixture.

### DETAILED DESCRIPTION

The preferred compounds of the invention have general formulae (I) and (II), and are further defined below. In the following description of these preferred compounds, the definitions for the various groups and variables represent the preferred definitions when they differ from those as broadly defined above, and are to be understood as independent of each other.

In a preferred embodiment, the 4-sulfide/sulfoxide/sulfonyl-1H-pyrazolyl derivative compounds or purified stereoisomers or stereoisomer mixtures of said compounds and their salts or prodrug forms thereof have structural formulas (I) or (II): 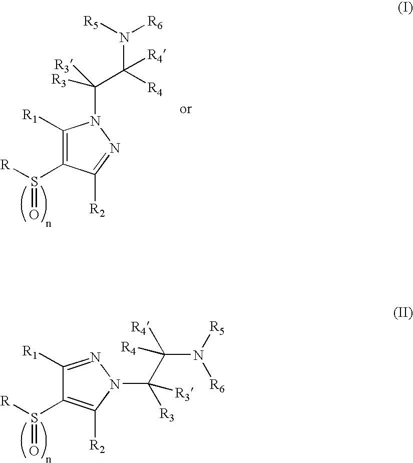
wherein:
n is 0, 1 or 2;
R is selected from the group consisting of:
   (a) (C₁-C₆)-alkyl optionally substituted by (C₃-C₈)-cycloalkyl,
   (b) (C₁-C₅)-alkenyl,
   (c) (C₁-C₅)-alkynyl, and
   (d) (C₆-C₁₀)-aryl which is optionally substituted with one to three substituents selected from the group consisting of:
      (d1) halogen,
      (d2) nitro,
      (d3) (C₁-C₅)-alkyl optionally substituted with halogen,
      (d4) (C₁-C₅)-alkenyl optionally substituted with (C₁-C₅)-alkyl,
      (d5) (C₁-C₅)-alkynyl optionally substituted with (C₁-C₅)-alkyl,
      (d6) (C₁-C₅)-alkoxy,
      (d7) NR₉C(═O)R₁₀,
      (d8) NR₉S(═O)ₙ—R₁₀,
      (d9) NR₁₁R₁₂,
      (d10) C(═O)NR₁₃R₁₄,
      (d11) (C₆-C₁₀)-aryl optionally substituted with one to three substituents selected from the group consisting of:
         (d11a) halogen,
         (d11b) (C₁-C₅)-alkyl,
         (d11c) (C₁-C₅)-alkoxy,
      (d12) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atom, and the other ring is a saturated or unsaturated three to eight membered cycloalkyl ring,
      (d13) OH, and
      (d14) CN;
R₁ and R₂ are independently selected from the group consisting of:
   (a) hydrogen, and
   (b) (C₁-C₅)-alkyl optionally substituted with halogen or hydroxy,
R₃, R_{3′}, R₄ and R_{4′} are independently selected from the group consisting of:
   (a) hydrogen, and
   (b) (C₁-C₅) alkyl;
R₃ and R₄ together form a four to eight membered saturated or unsaturated carbocyclic ring, or
R₄ and R₄, together form a (C₃-C₈)-cycloalkyl ring;
R₅ and R₆ are independently selected from the group consisting of hydrogen and (C₁-C₅)-alkyl;
R₉ is hydrogen or (C₁-C₅)-alkyl;
R₁₀ is selected from the group consisting of
   (a) (C₁-C₅)-alkyl optionally substituted with (C₃-C₈)-carbocyclic ring or (C₆-C₁₀)-aryl optionally substituted with halogen,
   (b) (C₃-C₈)-cycloalkyl optionally substituted with one to three substituents selected from the group consisting of halogen, (C₁-C₅)-alkoxy or (C₁-C₅)-alkyl optionally substituted by halogen,
   (c) (C₆-C₁₀)-aryl optionally substituted with one to three substituents selected from the group consisting of halogen, (C₁-C₅)-alkoxy and (C₁-C₅)-alkyl optionally substituted by halogen, and
   (d) —NR₁₁R₁₂;
R₁₁, R₁₂, R₁₃, R₁₄, R₁₇ and R₁₈ are independently selected from the group consisting of:
   (a) hydrogen,
   (b) (C₁-C₅)-alkyl,
   (c) (C₃-C₈)-cycloalkyl,
   (d) (C₆-C₁₀)-aryl, and
   (e) (C₆-C₁₀)-aryl-(C₁-C₅)-alkyl;
      or a purified stereoisomer or stereoisomer mixture of said compound, or salt of said compound, stereoisomer or stereoisomer mixture.

The more preferred compounds of the invention have general formulae (I) and (II), and are further defined below. In the following description of these more preferred compounds, the definitions for the various groups and variables represent the more preferred definitions when they differ from those as broadly defined above, and are to be understood as independent of each other.

In this more preferred embodiment, the 4-sulfide/sulfoxide/sulfonyl-1H-pyrazolyl derivative compounds or purified stereoisomers or stereoisomer mixtures of said compounds and their salts or prodrug forms thereof have structural formulas (I) or (II): 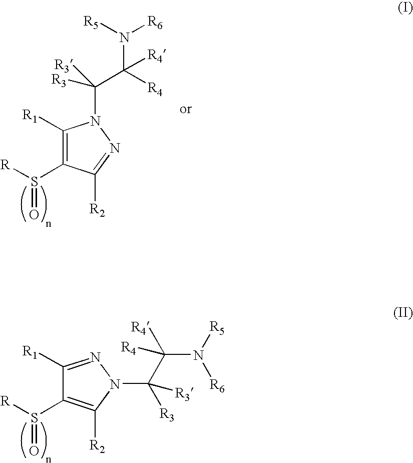
wherein:
n is 0, 1 or 2;
R is selected from the group consisting of:
   (a) (C₆-C₁₀)-aryl which is optionally substituted with one to three substituents selected from the group consisting of:
      (a1) halogen,
      (a2) nitro,
      (a3) (C₁-C₅)-alkyl optionally substituted with halogen,
      (a4) (C₁-C₅)-alkenyl optionally substituted with (C₁-C₅)-alkyl,
      (a5) (C₁-C₅)-alkynyl optionally substituted with (C₁-C₅)-alkyl,
      (a6) (C₁-C₅)-alkoxy,
      (a7) NR₉C(═O)R₁₀,
      (a8) NR₉S(═O)ₙ—R₁₀,
      (a9) NR₁₁R₁₂,
      (a10) C(═O)NR₁₃R₁₄,
      (a11) (C₆-C₁₀)-aryl optionally substituted with one to three substituents selected from the group consisting of:
         (a11a) halogen,
         (a11b) (C₁-C₅)-alkyl, and
         (a11c) (C₁-C₅)-alkoxy,
      (a12) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atom, and the other ring is a saturated or unsaturated three to eight membered cycloalkyl ring;
      (a13) OH, and
      (a14) CN;
R₁ and R₂ are independently selected from the group consisting of:
   (a) hydrogen, and
   (b) (C₁-C₅)-alkyl optionally substituted with halogen or hydroxy;
R₃, R_{3′}, R₄ and R_{4′} are independently selected from the group consisting of:
   (a) hydrogen, and
   (b) (C₁-C₅) alkyl;
R₅ and R₆ are independently selected from the group consisting of hydrogen and methyl,
R₉ is hydrogen or (C₁-C₅)-alkyl;
R₁₀ is selected from the group consisting of:
   (a) (C₁-C₅)-alkyl optionally substituted with (C₃-C₈)-carbocyclic ring or (C₆-C₁₀)-aryl optionally substituted with halogen,
   (b) (C₃-C₈)-cycloalkyl optionally substituted with one to three substituents selected from the group consisting of halogen, (C₁-C₅)-alkoxy or (C₁-C₅)-alkyl optionally substituted by halogen,
   (c) (C₆-C₁₀)-aryl optionally substituted with one to three substituents selected from the group consisting of halogen, (C₁-C₅)-alkoxy and (C₁-C₅)-alkyl optionally substituted by halogen, and
   (d) —NR₁₁R₁₂;
R₁₁, R₁₂, R₁₃, R₁₄, R₁₇ and R₁₈ are independently selected from the group consisting of:
   (a) hydrogen,
   (b) (C₁-C₅)-alkyl, and
   (c) (C₃-C₈)-cycloalkyl,
      or a purified stereoisomer or stereoisomer mixture of said compound, or salt of said compound, stereoisomer or stereoisomer mixture.

The compounds of the present invention may contain asymmetric centers on the molecule, depending upon the nature of the various substituents. Each such asymmetric center will produce two optical isomers. In certain instances, asymmetry may also be present due to restricted rotation about a central bond joining the two aromatic rings of the specified compounds. It is intended that all isomers, either by nature of asymmetric centers or by restricted rotation as described above, as separated, pure or partially purified isomers or racemic mixtures thereof, be included within the scope of the invention.

In cases in which the compounds have unsaturated carbon-carbon double bonds, both the cis (Z) and trans (E) isomers are within the scope of this invention.

In cases where the compounds may exist in tautomeric forms, each tautomeric form is contemplated as being encompassed by the scope of the invention whether existing in equilibrium with its corresponding tautomeric form or whether set in that form due through chemical derivatization.

Pharmaceutically acceptable salts of these compounds as well as commonly used prodrugs of these compounds are also within the scope of the invention.

Salts are especially the pharmaceutically acceptable salts of compounds of formulas (I) or (II) such as, for example, organic or inorganic acid addition salts of compounds of formulas (I) or (II). Suitable inorganic acids include but are not limited to halogen acids (such as hydrochloric acid), sulfuric acid, or phosphoric acid. Suitable organic acids include but are not limited to carboxylic, phosphonic, sulfonic, or sulfamic acids, with examples including acetic acid, trifluoroacetic acid, propionic acid, octanoic acid, decanoic acid, dodecanoic acid, glycolic acid, lactic acid, 2- or 3-hydroxybutyric acid, γ-aminobutyric acid (GABA), gluconic acid, glucosemonocarboxylic acid, benzoic acid, salicylic acid, phenylacetic acid, mandelic acid, methanesulfonic acid, trifluoromethanesulfonic acid, fumaric acid, oxalic acid, succinic acid, adipic acid, pimelic acid, suberic acid, azeiaic acid, malic acid, tartaric acid, citric acid, glucaric acid, galactaric acid, amino acids (such as glutamic acid, aspartic acid, N-methylglycine, acetytaminoacetic acid, N-acetylasparagine or N-acetylcysteine), pyruvic acid, acetoacetic acid, phosphoserine, and 2- or 3-glycerophosphoric acid.

In addition, pharmaceutically acceptable salts include acid salts of inorganic bases, such as salts containing alkaline cations (e.g., Li⁺ Na⁺ or K⁺), alkaline earth cations (e.g., Mg⁺², Ca⁺² or Ba⁺²), the ammonium cation, as well as acid salts of organic bases, including aliphatic and aromatic substituted ammonium, and quaternary ammonium cations such as those arising from protonation or peralkylation of triethylamine, N,N-diethylamine, N,N-dicyclohexylamine, pyridine, N,N-dimethylaminopyridine (DMAP), 1,4-diazabicyclo[2.2.2]octane (DABCO), 1,5-diazabicyclo[4.3.0]non-5-ene (DBN) and 1,8-diazabicyclo[5.4.0]undec-7-ene (DBU).

Prodrugs are considered to be any covalently bonded carriers which release the active parent compound of formula (I) or (II) in vivo. Formation of prodrugs is well known in the art in order to enhance the properties of the parent compound; such properties include solubility, absorption, biostability and release time (see “Pharmaceutical Dosage Form and Drug Delivery Systems” (Sixth Edition), edited by Ansel et al., publ. by Williams & Wilkins, pgs. 27-29, (1995) which is hereby incorporated by reference).

Commonly used prodrugs of the disclosed compounds of formulas (I) and (II) are designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Tenth Edition), editor Hardman et al., publ. by McGraw-Hill, pages 12-18, (2001), which is hereby incorporated by reference).

Definitions

The term “halogen” or “halo” as it appears in the specification and claims refers to fluorine, chlorine, bromine, and iodine substituents for the purposes of this invention. When halogen is a possible substituent on an alkyl group, the alkyl may be fully substituted, up to perhalo.

The term “fused bicyclo ring” as it appears in the specification and claims refers to a substituent which is a two ring structure which share two carbon atoms. The bonding between the fused bicyclo ring and the compound and/or atom to which it is attached can be through either of the two rings.

The term “spiro” ring as it appears in the specification and claims refers to a two ring system having one atom in common (e.g. a spiro ring attached to a phenyl group means that the spiro ring shared a carbon with the phenyl group).

Description of the Compositions

The invention also includes pharmaceutical compositions comprising one or more of the compounds of formulas (I) or (II), or a purified stereoisomer or stereoisomer mixture or their salt or prodrugs form thereof, with a pharmaceutically acceptable ingredient.

The invention also relates to pharmaceutical compositions containing a therapeutically effective amount of the compounds of formulas (I) and (II), or a purified stereoisomer or stereoisomer mixture or their salt or prodrug form thereof, and their use in combination with other drugs or therapies for the treatment of diseases and/or behaviors associated with the 5-HT_{2C} receptor.

The pharmaceutical compositions are prepared so that they may be administered orally, dermally, parenterally, nasally, ophthalmically, otically, sublingually, rectally or vaginally. Dermal administration includes topical application or transdermal administration. Parenteral administration includes intravenous, intraarticular, intramuscular, and subcutaneous injections, as well as use of infusion techniques. One or more compounds of the invention may be present in association with one or more non-toxic pharmaceutically acceptable ingredients and optionally, other active anti-proliferative agents, to form the pharmaceutical composition. These compositions can be prepared by applying known techniques in the art such as those taught in Remington's Pharmaceutical Sciences (Fourteenth Edition), Managing Editor, John E. Hoover, Mack Publishing Co., (1970) or Pharmaceutical Dosage Form and Drug Delivery Systems (Sixth Edition), edited by Ansel et al., publ. by Williams & Wilkins, (1995), each of which is hereby incorporated by reference.

Commonly used pharmaceutical ingredients which can be used as appropriate to formulate the composition for its intended route of administration include:
acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃)
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones and styrene-butadiene copolymers);
buffering agents (examples include but are not limited to potassium metaphosphate, potassium phosphate monobasic, sodium acetate, sodium citrate anhydrous and sodium citrate dihydrate)
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)
chelating agents (examples include but are not limited to edetate disodium and edetic acid)
colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyethylene 50 stearate);
encapsulating agents (examples include but are not limited to gelatin and cellulose acetate phthalate)
flavorants (examples include but are not limited to anise oil, cinnamon oil, cocoa, menthol, orange oil, peppermint oil and vanillin);
humectants (examples include but are not limited to glycerin, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)
plasticizers (examples include but are not limited to diethyl phthalate and glycerin);
solvents (examples include but are not limited to alcohol, corn oil, cottonseed oil, glycerin, isopropyl alcohol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan monopalmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methyl cellulose, tragacanth and veegum);
sweetening agents (examples include but are not limited to aspartame, dextrose, glycerin, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, povidone and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, povidone, sodium alginate and tragacanth); and
wetting agents (examples include but are not limited to heptadecaethylene oxycetanol, lecithins, polyethylene sorbitol monooleate, polyoxyethylene sorbitol monooleate, polyoxyethylene stearate,).

Depending on the route of administration, the compositions can take the form of aerosols, capsules, creams, elixirs, emulsions, foams, gels, granules, inhalants, lotions, magmas, ointments, peroral solids, powders, sprays, syrups, suppositories, suspensions, tablets and tinctures.

Optional additional agents which can be added to the composition include but are not limited to compounds which are known to treat obesity and obesity related disorder such as diabetes, abnormal feeding behavior, eating disorders (such as bulimia nervosa and anorexia nervosa) and premenstrual tension.

Examples of agents for treating obesity include appetite suppressants such as benzphetamine, diethylpropion, Mazindol, phendimetrazine and phentermine.

Examples of agents for treating diabetes include insulin for insulin-dependent diabetes (IDDM) and sulfonylurea compounds for non-insulin dependent diabetes (NIDDM). Examples of sulfonylureas include tolbutamide, chlorpropamide, tolazamide, acetohexamide, glycburide, glipizide and gliclazide.

It had previously been disclosed that psychosomatic disorders such as bulimia nervosa may respond at least partly to treatment with antidepressants such as tricyclic monoamine oxidase (MAO) inhibitors and serotonin reuptake inhibitors (see Goodman and Gilman's The Pharmacological Basis of Therapeutics (Tenth Edition), editor Hardman et al., publ. by McGraw-Hill, page 469, (2001), the contents of which is hereby incorporated by reference. Likewise it would be expected that these agents (e.g. fluoxetine) in combination with the applicants described compounds would have similar effects.

For all regimens of use disclosed herein for compounds of formulas (I) or (II), the daily oral dosage regimen will preferably be from 0.01 to 200 mg/kg of total body weight. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

It will be appreciated by those skilled in the art that the particular method of administration will depend on a variety of factors, all of which are considered routinely when administering therapeutics. It will also be understood, however, that the specific dose level for any given patient will depend upon a variety of factors, including, but not limited to the activity of the specific compound employed, the age of the patient, the body weight of the patient, the general health of the patient, the gender of the patient, the diet of the patient, time of administration, route of administration, rate of excretion, drug combinations, and the severity of the condition undergoing therapy. It will be further appreciated by one skilled in the art that the optimal course of treatment, i.e., the mode of treatment and the daily number of doses of a compound of formulas (I) or (II) or a pharmaceutically acceptable salt thereof given for a defined number of days, can be ascertained by those skilled in the art using conventional treatment tests.

Description of Preparative Methods

General Methods of Preparation of Formula I and II Compounds

Compounds of formulas I and II may generally be prepared by the route illustrated in Reaction Scheme I below. A 1,3-diketone of formula 3, either commercially available or readily prepared by well-known methods (e.g. condensation of esters), may be halogenated to the corresponding halo diketones of formula 4 (where X is halo) using standard conditions such as sulfuryl chloride in a suitable solvent, and can, in turn, S-alkylate a thiol of formula RSH, facilitated by a base such as pyridine or an inorganic carbonate. Alternatively, the diketone 3 may be allowed to react with a disulfide of formula RS—SR, facilitated by base, to provide the mercapto diester of formula 5. Reaction of the diester 5 with a substituted hydrazine gives a mixture compounds of formulas I and II directly. An alternative method is a two step sequence involving reaction of 5 with hydrazine to give the unsubstituted pyrazole of formula Ia. Alkylation of Ia in the presence of a base such as triethyl amine and a suitable reagent, R₅R₆NCH(R)CH(R₃)—X′, where X′ represents a leaving group such as halo, an arylsulfonate or an alkylsulfonate, provides a mixture of compounds of formulas I and II.

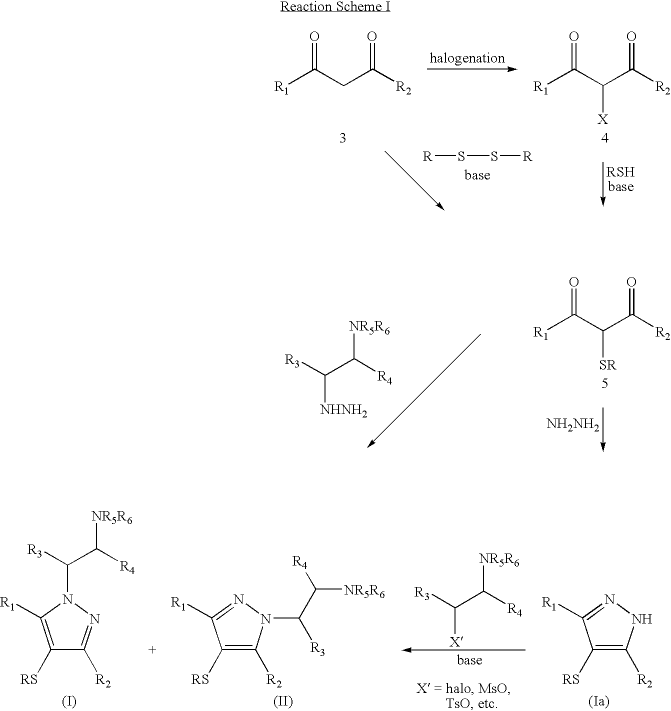

The choice of routes depends on the specific compound to be prepared and the availability of the starting materials. It is also understood that the R₅R₆NCH(R₄)CH(R₃)—NHNH₂ compound may be protected and deprotected (e.g., BocNH—CH₂CH₂NHNH₂) as needed in order to carry out the above Scheme. Oxidation of the RS group with an oxidative agent, such as hydrogen peroxide or MCPBA, to either the RSO— or RSO₂— groups may also be accomplished at an appropriate stage of the synthesis, utilizing protection/deprotection steps, if necessary.

In the foregoing and in the following examples, all temperatures are set forth uncorrected in degrees Celsius; and, unless otherwise indicated, all parts and percentages are by weight.

The entire disclosure of all applications, patents and publications, cited above or below, are hereby incorporated by reference.

### Abbreviations and Acronyms

When the following abbreviations are used herein, they have the following meaning:
Ac₂O acetic anhydride
anhy anhydrous
n-BuOH n-butanol
t-BuOH t-butanol
CD₃OD methanol-d₄
Celite® diatomaceous earth filter agent, ® Celite Corp.
CH₂Cl₂ methylene chloride
CI-MS chemical ionization mass spectroscopy
conc concentrated
dec decomposition
DME dimethoxyethane
DMF N,N-dimethylformamide
DMSO dimethylsulfoxide
ELSD evaporative light scattering detector
EtOAc ethyl acetate
EtOH ethanol (100%)
Et₂O diethyl ether
Et₃N triethylamine
HATU O-(7-azabenzotriazol-1-yl)-N,N,N′N′-tetramethyluronium hexafluorophosphate
HPLC ES-MS high performance liquid chromatography-electrospray mass spectroscopy
Ms methanesulfonyl (mesyl)
NMM 4-methylmorpholine
Ph₃P triphenylphosphine
Pd(OAc)₂ palladium acetate
RT retention time (HPLC0)
rt room temperature
THF tetrahydrofuran
TFA trifluoroacetic acid
TLC thin layer chromatography
Ts p-toluenesulfonyl (tosyl)

### EXPERIMENTAL EXAMPLES

All reactions were performed in flame-dried or oven-dried glassware under a positive pressure of dry argon, and were stirred magnetically unless otherwise indicated. Sensitive liquids and solutions were transferred via syringe or cannula, and introduced into reaction vessels through rubber septa. Commercial grade reagents and solvents were used without further purification. Thin layer chromatography (TLC) was performed on Analtech UNIPLATE™ pre-coated glass-backed silica gel 60 A F-254 250 μm plates. Column chromatography (flash chromatography) was performed on a Biotage system using 32-63 micron, 60 A, silica gel pre-packed cartridges. Proton (¹H) nuclear magnetic resonance (NMR) spectra were measured with a Varian (300 MHz) spectrometer with residual protonated solvent (CHCl₃ δ 7.26; MeOH δ 3.30; DMSO δ 2.49) as standard. Low-resolution mass spectra (MS) were either obtained as electron impact (EI) mass spectra or as fast atom bombardment (FAB) mass spectra. HPLC-electrospray mass spectra (HPLC ES-MS) were obtained using a Hewlett-Packard 1100 HPLC equipped with a quaternary pump, a variable wavelength detector, a YMC Pro C18 2.0 mm×23 mm column, and a Finnigan LCQ ion trap mass spectrometer with electrospray ionization. Gradient elution from 90% A to 95% B over 4 minutes was used on the HPLC. Buffer A was 98% water, 2% Acetonitrile and 0.02% TFA. Buffer B was 98% Acetonitrile, 2% water and 0.018% TFA. Spectra were scanned from 140-1200 amu using a variable ion time according to the number of ions in the source.

The IUPAC name was obtained using the ACD/ILab Web service.

### EXPERIMENTAL

### Example 1

### Preparation of 2-{4-[(4′-chloro-1,1′-biphenyl-4-yl)sulfanyl]-3,5-diethyl-1H-pyrazol-1-yl}ethylamine

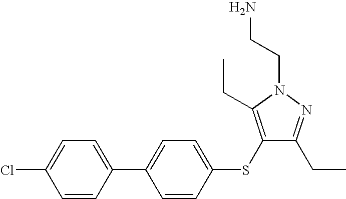

### Step 1. Preparation of 4-chloro 3,5-heptadione

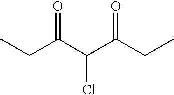

A solution of sulfuryl chloride (6.29 mL, 0.0783 mol), in toluene (20 mL) was added dropwise to a solution of 3,5-heptadione (10.04 g, 0.0783 mol) in toluene (100 mL, 0.78 M) and the resulting yellow solution stirred at room temperature for 18 h and concentrated to a yellow oil (11.26 g, 88%). GC/MS 163 (M+, 100%), ¹H NMR (300 MHz, CDCl₃) δ 2.64 (m, 4H), 1.16 (m, 6H).

### Step 2. Preparation of 4-[4-bromophenyl] 4-sulfanyl-heptane 3,5-dione

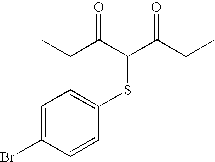

Pyridine (0.27 mL, 3.40 mmol) was added very slowly to a mixture of 4-chloro 3,5-heptadione (0.5 g, 3.08 mmol) and 4-bromothiophenol (0.581 g, 3.08 mmol) and the resulting slurry was stirred at room temperature for 3 h. The mixture was diluted with ether and filtered, and then the filtrate was concentrated to give yellow oil. (0.945 g, 98%, used for the next step without further purification). ¹H NMR (300 MHz, CDCl₃) δ 7.38 (d, 2H), 6.94 (d, 2H), 2.74-2.62 (m, 4H), 1.09 (t, 6H).

### Step 3. Preparation of 3,5-diethyl 4-[4-bromophenyl] sulfanyl-1H-pyrazole

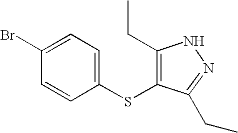

Hydrazine (0.14 mL, 4.50 mmol), was added to a mixture of the product prepared in Step 2 above (0.945 g, 3.00 mmol) and acetic acid (2 drops) in ethanol (15 mL). The mixture was stirred at room temperature for 4 h and concentrated. The product (0.91 g, 97%) was isolated by column chromatography (30% EtOAc in Hexanes). R=0.42 (30% EtOAc in Hexanes), MS (Electronspray) 313 (M+2)⁺, ¹H NMR (300 MHz, CDCl₃) δ 7.29 (d, 2H), 6.83 (d, 2H), 2.65 (q, 4H), 1.19 (t, 6H).

### Step 4. Preparation of 4-[(4′-chloro-1,1′-biphenyl-4-yl)sulfanyl]-3,5-diethyl-1H-pyrazole

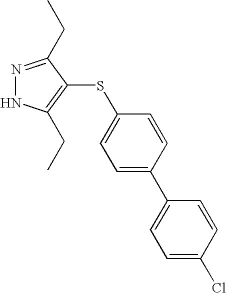

A mixture of the product prepared in Step 3 above (0.2 g, 0.643 mmol), 4-chlorophenyl boronic acid (0.2 g, 1.29 mmol), PdCl₂(PPh₃)₂ (0.009 g, 0.0129 mmol) and Na₂CO₃ (1.3 mL, 2N) in toluene (3 mL) was heated at 90° C. for 18 h and cooled to room temperature. Some ice was added and the mixture extracted with dichloromethane (3×20 mL) and dried over MgSO₄ and concentrated. The product was isolated by column chromatography (50% hexane in EtOAc) to give a cream colored solid (0.18 g, 82%). R=0.48 (50% hexane in EtOAc), GC/MS 345 (M+2)⁺, ¹H NMR (300 MHz, CDCl₃) δ 7.45-7.34 (m, 6H), 7.03 (d, 2H), 3.49 (q, 2H), 2.71 (q, 2), 1.28-1.21 (m, 6H).

### Step 5. Preparation of 2-{4-[(4′-chloro-1,1′-biphenyl-4-yl)sulfanyl]-3,5-diethyl-1H-pyrazol-1-yl}ethylamine

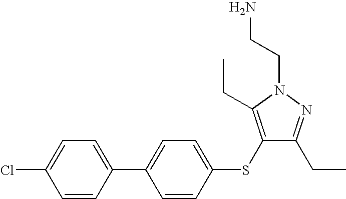

To a suspension of the product obtained in step 5 above (0.3 g, 0.875 mmol) in acetonitrile (1.5 mL) was added sodium hydroxide (0.14 g, 3.50 mmol). The mixture was stirred under argon for 30 min at room temperature. 2-Chloroethylamine hydrochloride (0.122 g, 1.05 mmol) was added, followed by tetrabutylammonium hydrogen sulfate (0.012 g, 0.0399 mol), the reaction mixture was stirred at reflux for 3 h and diluted with ethyl acetate (20 mL) dried over Na₂SO₄. The mixture was filtered and the filtrate concentrated. The residue was dissolved in ethyl acetate (35 mL) and filtered through a silica gel plug, using ethyl acetate as the initial eluant, then 5% methanol in ethyl acetate and finally 10% methanol in ethyl acetate. The eluants were concentrated to give 0.245 g, 72% of product. MS (Electrospray) 386 (M+H)⁺, ¹HNMR (300 MHz, CDCl₃) 7.46-7.35 (m, 6H), 7.02 (d, 2H), 4.14 (t, 2H), 3.21 (t, 2H), 2.73 (q, 2H), 2.62 (q, 2H), 1.22-1.09 (m, 61).

The hydrochloride salt was prepared by dissolving the product (0.245 g, 0.635 mmol) in ether (3 mL) and treatment with HCl in ether (6.35 mL, 1 M). The mixture was stirred for 1 h and concentrated. The residue was washed with ether (2×15 mL) and dried under vacuum to give a sticky solid (0.29 g, 100.%). ¹H NMR (300 MHz, DMSO) 7.63-7.44 (m, 6H), 7.06 (d, 2H), 4.37 (t, 2H), 3.24 (q, 2H), 2.71 (q, 2H), 2.49 (q, 2H), 1.12-1.00 (m, 6H).

### Example 2

### Preparation of 2-[3,5-dimethyl-4-(phenylsulfanyl)-1H-pyrazol-1-yl]ethylamine

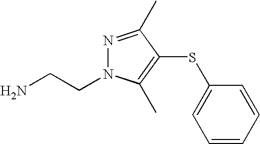

The desired compound was prepared by the same process as used for Example 1, starting from pentane 2,4-dione:

Pale yellow oil (0.406 g, 77%). R_{f}=0.12 (EtOAc), GC/MS 247 (M)⁺, ¹H NMR (300 MHz, CDCl₃) δ 7.27-6.96 (m, 5H), 4.13 (t, 2H), 3.19 (t, 2H), 2.29 (s, 3H), 2.2 (s, 3H).

HCl salt: Pale yellow solid. Mp 185-188° C., ¹H NMR (300 MHz, D₂O) δ 7.26-6.97 (m, 5H), 4.31 (t, 21H), 3.22 (q, 2H), 2.25 (s, 3H), 2.06 (s, 3H).

### Example 3

### Preparation of 2-[3,5-diethyl-4-(phenylsulfanyl)-1H-pyrazol-1-yl]ethylamine

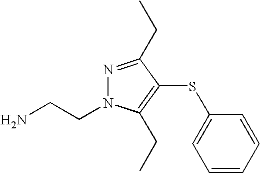

The compound was prepared using the same procedure described for Example 1. Product (0.348 g, 83%): ¹H NMR (300 MHz, CDCl₃) δ 7.45-7.37 (m, 5H), 4.11 (t, 2H), 3.18 (t, 2H), 2.70 (q, 21), 2.59 (q, 2H), 1.16 (t, 3H), 1.08 (t, 3H). HCl salt: (0.452 g, 85%). ¹H NMR (300 MHz, DMSO) δ 7.24-6.94 (m, 5H), 4.35 (t, 2H), 3.21 (q, 2H), 2.70 (q, 2H), 2.67 (q, 2H), 2.44 (q, 2H), 1.08-0.95 (t, 6H).

### Example 4

### Preparation of 2-{3,5-diethyl-4-[(4-nitrophenyl)sulfanyl]-1H-pyrazol-1-yl}ethylamine

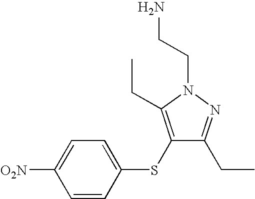

The compound was prepared using the same procedure described for as the procedure for Example 1. Product (13.12 g, 93%): MS (Electronspray) 321 (M+H)⁺, ¹H NMR (300 MHz, CDCl₃) δ 8.04 (d, 2H), 7.05 (d, 2H), 4.13 (t, 2H), 3.21 (t, 2H), 2.69 (q, 2H), 2.57 (q, 2H), 1.19-1.07 (m, 6H).

### Example 5

### Preparation of 2-{3,5-diethyl-4-[(4-nitrophenyl)sulfanyl]-1H-pyrazol-1-yl}ethylamine, Compound with Maleic Acid (1:1)

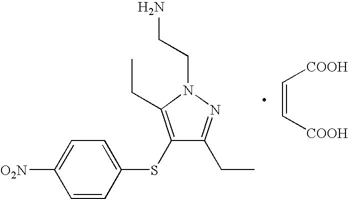

A solution of maleic acid (0.188 g, 1.62 mmol) in ether (5 mL) was added to a solution of free amine of Example 4 (0.45 g, 1.40 mmol) in ether (5 mL). The mixture was stirred under argon for 30 min and ether removed with a syringe. The residue was washed twice with ether (10 mL) and dried under vacuum to give 0.55 g, 90% of product: mp. 159-161° C., ¹H NMR (300 MHz, DMSO) δ 8.08 (d, 2H), 7.19 (d, 2H), 6.03 (s, 2H), 4.30 (t, 2H), 3.27 (t, 2H), 2.65 (q, 2H), 2.46 (q, 2H), 1.08 (t, 3H), 1.00 (t, 3H).

### Example 6

### Preparation of tert-butyl 2-{3,5-diethyl-4-[(4-nitrophenyl)sulfanyl]-1H-pyrazol-1-yl}ethylcarbamate

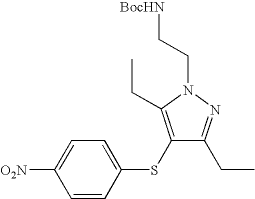

Di-tert-butyl dicarbonate (7.16 g, 0.0328 mol) was added in one portion to a solution of the compound prepared in Example 4 (10.21 g, 0.0319 mol) in dichloromethane (70 mL). The mixture was stirred at room temperature for 1 h and concentrated to give 13.40 g, 100% of bright yellow solid as the product. R_{f}=0.55 (50% EtOAc in Hexane), MS (Electronspray) 421 (M+H)⁺, ¹H NMR (300 MHz, CDCl₃) δ 8.05 (d, 2H), 7.06 (d, 2H), 4.97 (t, 1H), 4.19 (t, 2H), 3.61 (q, 2H), 2.66 (q, 2H), 2.56 (q, 2H), 1.43 (s, 9H), 1.17 (t, 3H), 1.08 (t, 3H).

### Example 7

### Preparation of tert-butyl 2-{4-[(4-aminophenyl)sulfanyl]-3,5-diethyl-1H-pyrazol-1-yl}ethylcarbamate

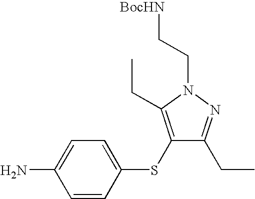

A solution of the compound prepared in Example 6 (5.3 g, 0.0126 mol) in ethyl acetate (150 mL) was subjected to hydrogenation using 10% palladium on carbon (0.53 g) at 50 psi of hydrogen for 24 h to give 4.52 g, 92% of pale yellow solid as product. R=0.35 (50% EtOAc in Hexane), MS (Electronspray) 391 (M+H)⁺, ¹H NMR (300 MHz, CDCl₃) δ 6.88 (d, 2H), 6.56 (d, 2H), 5.00 (t, 1H), 4.14 (t, 2H), 3.58 (q, 2H), 2.70 (q, 2H), 2.62 (q, 2H), 1.44 (s, 9H), 1.18 (t, 3H), 1.08 (t, 3H).

TFA salt: To a solution of above product (0.10 g, 0.256 mmol) in CH₂Cl₂ (0.5 mL) was added trifluoroacetic acid (1 mL). The mixture was stirred for 2 h and concentrated under reduced pressure to give a viscous yellow oil (0.104 g, 64%). ¹H NMR (300 MHz, CDCl₃) δ 6.99 (s, 4H), 4.27 (t, 2H), 3.25 (q, 2H), 2.67 (q, 2H), 2.48 (q, 2H), 1.07 (t, 3H), 0.99 (t, 3H).

### Example 8

### Preparation of tert-butyl 2-[4-({4-[(cyclopropylcarbonyl)amino]phenyl}sulfanyl)-3,5-diethyl-1H-pyrazol-1-yl]ethylcarbamate

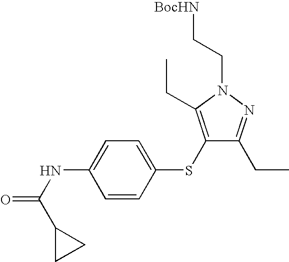

Cyclopropanecarbonyl chloride (0.022 mL, 0.238 mmol) was added to a mixture of the free base prepared in Example 7 (0.09 g, 0.231 mmol) and triethylamine (0.065 mL, 0.462 mmol) in dichloromethane (1 mL) at room temperature. The mixture was stirred for 5 h, diluted with dichloromethane (15 mL), washed with water (5 mL), dried over MgSO₄ and concentrated to give a bright yellow solid (0.1 g, 94%, used in the next step without further purification): R_{f}=0.25, MS (Electronspray) 459 (M+H)⁺, ¹H NMR (300 MHz, CDCl₃) δ 7.35 (d, 2H), 6.93 (d, 2H), 4.99 (t, 1H), 4.15 (t, 2H), 3.59 (q, 2H), 2.67 (q, 2H), 2.58 (q, 2H), 1.44 (s, 9H), 1.16 (t, 31H), 1.09-1.04 (m, 6H), 0.85-0.82 (m, 2H).

### Example 9

### Preparation of N-(4-{[1-(2-aminoethyl)-3,5-diethyl-1H-pyrazol-4-yl]sulfanyl}phenyl)propanamide, Trifluoroacetic Acid Salt

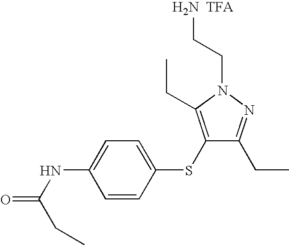

The compound was prepared using the same procedure described for the TFA salt in Example 7. Product (0.12 g, 100%): Mp. 209-211° C., ¹H NMR (300 MHz, CD₃OD) δ 7.39 (d, 2H), 6.94 (d, 2H), 4.35 (t, 2H), 3.43 (t, 2H), 2.76 (q, 2H), 2.58 (q, 2H), 1.78-1.66 (m, 1H), 1.16-1.03 (m, 6H), 0.96-0.77 (m, 4H).

### Example 10

### Preparation of tert-butyl 2-[3,5-diethyl-4-({4-[(methylsulfonyl)amino]phenyl}sulfanyl)-1H-pyrazol-1-yl]ethylcarbamate

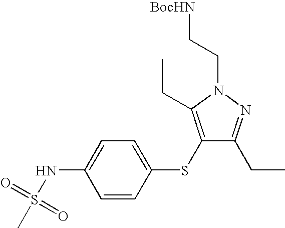

Methanesulfonyl chloride (0.059 mL, 0.757 mmol) was added to mixture of the free base prepared in Example 7 (0.29 g, 0.743 mmol) and pyridine (0.12 mL, 0.149 mmol) in dichloromethane (3 mL) at room temperature. The mixture was stirred for 2 h, diluted with dichloromethane (15 mL), washed with water (5 mL), dried over MgSO₄ and concentrated. The product was isolated by column chromatography (50% Hexane in EtOAc) to give a brown solid (0.26 g, 75%). R_{f}=0.29 (50% EtOAc in Hexane), MS (Electronspray): 469 (M+H)⁺, ¹H NMR (300 M[Hz, CDCl₃) δ 7.09 (d, 2H), 6.92 (d, 2H), 5.02 (t, 1H), 4.20 (t, 2H), 3.62 (q, 2H), 2.98 (s, 3H), 2.69 (q, 2H), 2.60 (q, 2H), 1.44 (s, 9H), 1.18 (t, 3H), 1.09 (t, 3H).

TFA salt: (0.23 g, 65%): ¹H NMR (300 MHz, CD₃OD) δ 7.12 (d, 2H), 6.99 (d, 2H), 4.37 (t, 2H), 3.45 (t, 2H), 2.91 (s, 3H), 2.77 (q, 2H), 2.60 (q, 2H), 1.16 (t, 3H), 1.10 (t, 3H).

### Example 11

### Preparation of tert-butyl 2-(3,5-diethyl-4-{[4-(methylamino)phenyl]sulfanyl}-1H-pyrazol-1-yl)ethylcarbamate

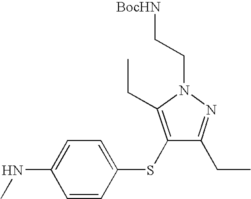

To a solution of sodium methoxide (0.138 g, 2.56 mmol) in anhydrous methanol (2 mL) was added a solution of the free base prepared in Example 7 (0.4 g, 1.02 mmol) in methanol (2 mL). The resultant mixture was added to a suspension of parafomaldehyde (0.31 g, 10.2 mmol) in methanol (2 mL). The mixture was stirred at room temperature for 2 h. Sodium borohydride (0.116 g, 3.07 mmol) was added in one portion and the mixture stirred at room temperature for 2 h, quenched with 1N sodium hydroxide (2 mL) and extracted with dichloromethane (2×30 mL). The combined organic extracts were dried over Na₂SO₄ and concentrated under reduced pressure. The product was isolated by column chromatography (50% Hexane in EtOAc) to give 0.162 g, 39% of product. MS (Electronspray) 405 (M+H)⁺, ¹H NMR (300 MHz, CDCl₃) δ 6.94 (d, 2H), 6.50 (d, 2H), 5.01 (t, 1H), 4.13 (t, 2H), 3.58 (q, 2H), 2.80 (s, 3H), 2.71 (q, 2H), 2.63 (q, 2H), 1.44 (s, 9H), 1.18 (t, 3H), 1.09 (t, 3H).

TFA salt: The compound was prepared using the same procedure described for TFA salt of Example 7. Product (0.07 g, 83%): ¹H NMR (300 MHz, CD₃OD) δ 7.23 (d, 2H), 7.12 (d, 2H), 4.39 (t, 2H), 3.46 (t, 2H), 2.98 (s, 3H), 2.76 (q, 2H), 2.58 (q, 2H), 1.16 (t, 3H), 1.09 (t, 3H).

### Example 12

### Preparation of tert-butyl 2-(4-{[4-(dimethylamino)phenyl]sulfanyl}-3,5-diethyl-1H-pyrazol-1-yl)ethylcarbamate (37)

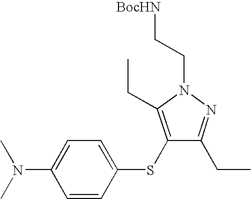

The compound was prepared using the same procedure described for as the procedure for Example 11. Product (0.21 g, 49%): MS (Electronspray) 419 (M+H)⁺, ¹H NMR (300 MHz, CDCl₃) δ 6.98 (d, 2H), 6.62 (d, 2H), 5.01 (t, 1H), 4.14 (t, 2H), 3.59 (q, 2H), 2.90 (s, 6H), 2.72 (q, 2H), 2.64 (q, 2H), 1.45 (s, 9H), 1.19 (t, 3H), 1.09 (t, 3H).

TFA salt: The compound was prepared using the same procedure described for Example 7, TFA salt Product (0.196 g, 100%): ¹H NMR (300 MHz, CD₃OD) δ 7.36 (d, 2H), 7.15 (d, 2H), 4.40 (t, 2H), 3.50-3.45 (m, 2H), 3.21 (s, 6H), 2.77 (q, 2H), 2.59 (q, 2H), 1.19-1.08 (m, 6H).

### Example 13

### Preparation of tert-butyl 2-[4-({4-[(2,2-dimethylpropanoyl)(methyl)amino]phenyl}sulfanyl)-3,5-diethyl-1H-pyrazol-1-yl]ethylcarbamate

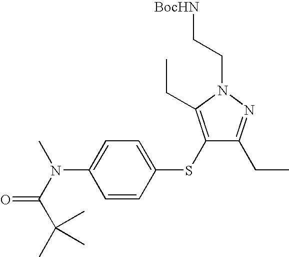

The compound was prepared using the same procedure described for Example 8. Product (0.16 g, 92%): R_{f}=0.38 (50% EtOAc in Hexane). MS (Electronspray) 489 (M+H)⁺. ¹H NMR (300 MHz, CDCl₃) δ 7.02 (d, 2H), 6.96 (d, 2H), 5.00 (t, 1H), 4.16 (t, 2H), 3.59 (q, 2H), 3.15 (s, 3H), 2.68 (q, 2H), 2.58 (q, 2H), 1.42 (s, 9H), 1.15 (t, 3H), 1.07 (t, 3H), 1.00 (s, 9H).

TFA salt: The compound was prepared using the same procedure described in Example 7. Product (0.075 g, 44%): ¹H NMR (300 MHz, CD₃OD) δ 7.15 (d, 2H), 7.05 (d, 2H), 4.39 (t, 2H), 3.4.6 (q, 2H), 3.30 (s, 3H), 2.78 (q, 2H), 2.60 (q, 2H), 1.19-1.08 (s, 6H), 1.01 (s, 9H).

### Example 14

### Preparation of methyl 4-[(1-{2-[(tert-butoxycarbonyl)amino]ethyl}-3,5-diethyl-1H-pyrazol-4-yl)sulfanyl]benzoate

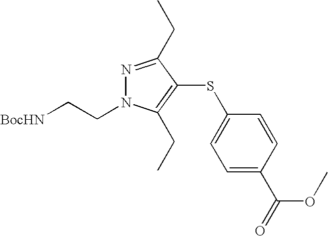

The compound was prepared as in Example 1, starting from ethyl 4-mercaptobenzoate\ and the product of Step 1, Example 1. Cesium carbonate (3.9 g, 11.9 mmol) was added to a solution of hydrazine (1.16 g, 3.99 mmol) and 2-(bromoethyl)-carbamic acid tert-butyl ester (1.61 g, 7.19 mmol) in N,N′-dimethylformamide (27 mL). The mixture was stirred at room temperature for 16 h and diluted with ethyl acetate and washed with water (20 mL) and dried over MgSO₄ and concentrated. The product (1.49 g, 86%) was isolated by column chromatography (45% EtOAc in Hexane). R=0.50 (50% EtOAc in Hexane), MS (Electronspray) 434 (M+H)⁺, ¹H NMR (300 MHz, CDCl₃) δ 7.84 (d, 2H), 6.99 (d, 2H), 4.99 (t, 1H), 4.18 (t, 2H), 3.87 (s, 3H), 3.61 (q, 2H), 2.66 (q, 4H), 2.57 (q, 2H), 1.43 (s, 9H), 1.16 (t, 3H), 1.07 (t, 3H).

### Example 15

### Preparation of methyl 4-{[1-(2-aminoethyl)-3,5-diethyl-1H-pyrazol-4-yl]sulfanyl}benzoate, Trifluoroacetic Acid Salt

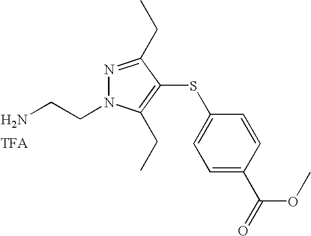

The compound was prepared by the procedure for the TFA salt of Example 7. Product (0.239 g, 98%): ¹H NMR (300 MHz, CD₃OD) δ 7.81 (d, 2H), 7.03 (d, 2H), 4.37 (t, 2H), 3.83 (s, 3H), 3.45 (t, 2H), 2.73 (q, 4H), 2.55 (q, 2H), 1.13 (t, 3H), 1.07 (t, 3H).

### Example 16

### Preparation of 4-[(1-{2-[(tert-butoxycarbonyl)amino]ethyl}-3,5-diethyl-1H-pyrazol-4-yl)sulfanyl]benzoic Acid

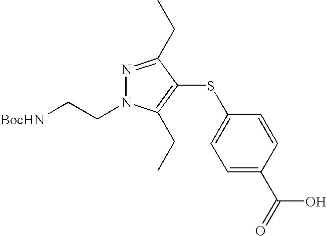

Lithium hydroxide (17 mL 1 N) was added to a solution of Example 14 (1.49 g, 3.44 mmol) in dimethoxyethane (20 mL). The cloudy solution was stirred for 2.5 h and concentrated. The residue was washed with dichloromethane (2 ×10 mL) and dissolved in water (10 mL) and acidified to pH=5 with 10% citric acid. The mixture was extracted with ethyl acetate (3×25 mL) and the extract dried over Na₂SO₄ and concentrated to give a white solid (1.38 g, 96%). R=0.17 (66% EtOAc in Hexane), MS (Electronspray) 420 (M+H)⁺, ¹H NMR (300 MHz, CDCl₃) δ 7.85 (d, 2H), 6.84 (d, 2H), 5.02 (t, 1H), 4.18 (t, 2H), 3.61 (t, 2H), 2.67-2.56 (m, 4H), 1.27 (s, 9H), 0.91 (t, 6H).

### Example 17

### Preparation of 4-[(3,5-diethyl-1-propyl-1H-pyrazol-4-yl)sulfanyl]benzoic acid, Trifluoroacetic Acid Salt

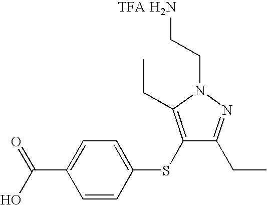

The compound was prepared by the procedure for the TFA salt of Example 7. Product (0.097 g, 94%): ¹H NMR (300 MHz, CD₃OD) δ 7.83 (d, 2H), 7.04 (d, 2H), 4.39 (t, 2H), 3.46 (t, 2H), 2.74 (q, 2H), 2.57 (q, 2H), 1.15 (t, 3H), 1.09 (t, 3H).

### Example 18

### Preparation of tert-butyl 2-[4-({4-[(cyclopropylamino)carbonyl]phenyl}sulfanyl)-3,5-diethyl-1H-pyrazol-1-yl]ethylcarbamate

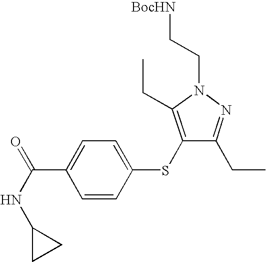

Cyclopropylamine (0.343 mL, 4.85 mmol) was added to a solution of the compound prepared in Example 16 (0.407 g, 0.97 mmol), N-methylmorpholine (0.107 mL, 0.97 mmol) and O-(7-azabenzotiazole-1-yl)-N,N,N,N-tetramethyluronium hexafluorophosphate (0.494 g, 1.26 mmol) in dichloromethane (3 mL). The mixture was stirred at room temperature for 16 h and concentrated. The product (0.359 g, 81%) was isolated by column chromatography (66% EtOAc in Hexane). R_{f} 0.35 (66% EtOAc in Hexane), MS (Electronspray) 459 (M+H)⁺. ¹H NMR (300 MHz, CDCl₃) δ 7.51 (d, 2H), 6.94 (d, 2H), 4.94 (t, 1H), 4.14 (t, 2H), 3.56 (q, 2H), 2.84-2.81 (m, 1H), 2.61 (q, 2H), 2.52 (q, 2H), 1.39 (s, 9H), 1.11 (t, 3H), 0.89-0.78 (m, 2H), 0.60-0.52 (m, 2H).

### Example 19

### Preparation of N-cyclopropyl-4-[(3,5-diethyl-1-propyl-1H-pyrazol-4-yl)sulfanyl]benzamide, Trifluoroacetic Acid Salt

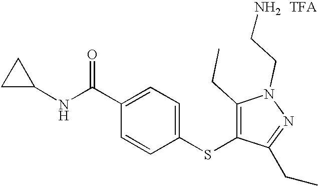

The compound was prepared by the procedure for the TFA salt of Example 7 Product (0.118 g, 98%): ¹H NMR (300 MHz, CD₃OD) δ 7.64 (d, 2H), 7.03 (d, 2H), 4.39 (t, 2H), 3.47 (t, 2H), 2.84-2.71 (m, 3H), 2.58 (q, 2H), 1.18 (t, 3H), 1.09 (t, 3H), 0.82-0.76 (m, 2H), 0.63-0.58 (m, 2H).

### Example 20

### Preparation of tert-butyl 2-[4-(1,3-benzoxazol-2-ylsulfanyl)-3,5-diethyl-1H-pyrazol-1-yl]ethylcarbamate

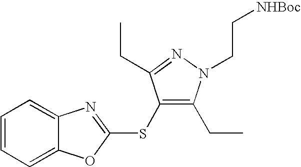

The compound was prepared by the procedure described for Example 14. Product (0.94 g, 69%). R_{f}=0.40 (50% EtOAc in Hexane), MS (Electronspray) 417 (M+H)⁺, ¹H NMR (300 MHz, CDCl₃) δ 7.58-7.55 (m, 1H), 7.41-7.37 (m, 1H), 7.25-7.21 (m, 2H), 5.06 (t, 1H), 4.17 (t, 2H), 3.61 (q, 2H), 2.80-2.66 (m, 4H), 1.43 (s, 9H), 1.23 (t, 3H), 1.16 (t, 3H).

### Example 21

### Preparation of 2-[(3,5-diethyl-1-propyl-1H-pyrazol-4-yl)sulfanyl]-1,3-benzoxazole, Trifluoroacetic Acid Salt

The compound was prepared by the procedure for the TFA salt of Example 7 Product (0.314 g, 84%): ¹H NMR (300 MHz, CDCl₃) δ 7.54-7.46 (m, 2H), 7.33-7.30 (m, 2H), 4.43 (t, 2H), 3.51 (q, 2H), 2.83 (q, 2H), 2.67 (q, 2H), 1.21 (t, 3H), 1.17 (t, 3H).

### Example 22

### Preparation of tert-butyl 2-{3,5-diethyl-4-[(4-nitrophenyl)sulfanyl]-1H-pyrazol-1-yl}ethyl(methyl)carbamate

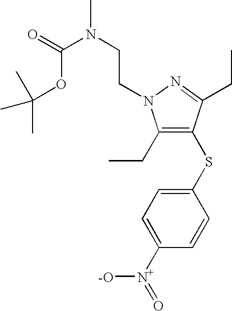

Methanesulfonyl chloride (0.39 mL, 5.02 mmol) was added to a cooled (0° C.) solution of tert-butyl 3-hydroxypropyl(methyl)carbamate (0.8 g, 4.57 mmol) and triethylamine (0.76 mL, 5.48 mmol) in dichloromethane (10 mL). The resulting cloudy mixture was stirred at 0° C for 30 min and concentrated. The residue was taken up in ethyl acetate (20 mL) and filtered through a plug of silica gel. The filtrate was concentrated and dissolved in N,N′-dimethylformamide (3 mL), and the solution added to a mixture of Example 4 (1.27 g, 4.57 mmol) and sodium hydride (0.274 g, 6.85 mmol, 60%) in N,N′-dimethylformamide (7 mL). The resulting dark golden yellow suspension was heated at 50° C. for 15 h, cooled and diluted with ethyl acetate (50 mL) and water (10 mL). The organic washed with water (2×10 mL), dried over MgSO₄ and concentrated. The product (1.25 g, 63%) was isolated by column chromatography (50% EtOAc in Hexane). R_{f}=0.49 (50% EtOAc in Hexane), MS (Electronspray) 435 (M+H)⁺, ¹H NMR (300 MHz, CDCl₃) δ 8.04 (d, 2H), 7.05 (d, 2H), 4.26-4.20 (m, 2H), 3.67 (t, 2H), 2.70-2.52 (m, 7H), 1.44 (s, 9H), 1.15 (t, 3H), 1.07 (t, 3H).

### Example 23

### Preparation of tert-butyl 2-{4-[(4-aminophenyl)sulfanyl]-3,5-diethyl-1H-pyrazol-yl}ethyl(methyl)carbamate

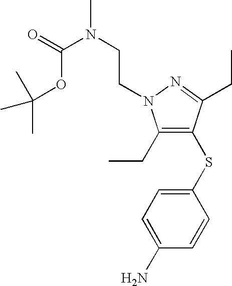

The compound was prepared by the reduction procedure described in Example 7. Product (0.72 g, 93%): R=0.42 (50% EtOAc in Hexane), MS (Electronspray) 405 (M+H)⁺, ¹H NMR (300 MHz, CDCl₃) δ 6.87 (d, 2H), 6.57 (d, 2H), 4.19-4.13 (m, 2H), 3.62 (q, 2H), 2.71-2.53 (m, 7H), 1.45 (s, 9H), 1.15 (t, 3H), 1.06 (t, 3H).

### Example 24

### Preparation of tert-butyl 2-[4-({4-[(2,2-dimethylpropanoyl)amino]phenyl}sulfanyl)-3,5-diethyl-1H-pyrazol-1-yl]ethyl(methyl)carbamate

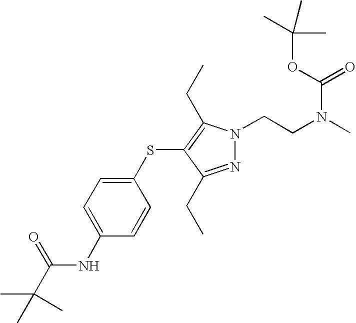

The compound was prepared by the procedure for Example 8. Product (0.42 g, 99%): R_{f}=0.33 (50% EtOAc in Hexane), MS (Electronspray) 489 (M+H)⁺, ¹H NMR (300 MHz, CDCl₃) δ 7.35 (d, 2H), 6.92 (d, 2H), 4.22-4.17 (m, 2H), 3.63 (q, 2H), 2.67-2.54 (m, 7H), 1.44 (s, 9H), 1.28 (s, 9H), 1.14 (t, 3H), 1.05 (t, 3H).

### Example 25

### Preparation of N-[4-({3,5-diethyl-1-[2-(methylamino)ethyl]-1H-pyrazol-4-yl}sulfanyl)phenyl]-2,2-dimethylpropanamide, Trifluoroacetic Acid Salt

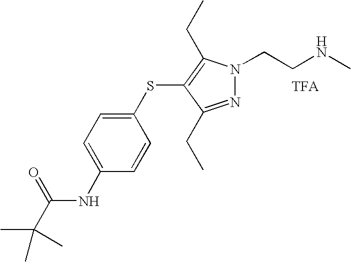

The compound was prepared by the procedure for the TFA salt of Example 7. Product (0.43 g, 99%): MS (Electronspray) 389 (M+H)⁺, ¹H NMR (300 MHz, CD₃OD) δ 7.39 (d, 2H), 6.96 (d, 2H), 4.42 (t, 2H), 3.53 (t, 2H), 2.81 (s, 3H), 2.77 (q, 2H), 2.60 (q, 2H), 1.26 (s, 9H), 1.16 (t, 3H), 1.10 (t, 3H).

### Example 26

### Preparation of tert-butyl 2-{3,5-diethyl-4-[(5-nitro-2-pyridinyl)sulfanyl]-1H-pyrazol-1-yl}ethylcarbamate

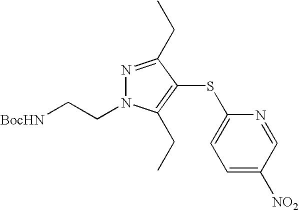

The compound was prepared by the procedure for Example 14. Product (4.33 g, 95%). R_{f}=0.46 (50% EtOAc in Hexane), MS (Electronspray) 422 (M+H)⁺, ¹H NMR (300 MHz, CDCl₃) δ 9.21 (dd, 1H), 8.20 (dd, 1H), 6.91 (d, 1H), 4.97 (t, 1H), 4.20 (t, 2H), 3.61 (q, 2H), 2.68 (q, 2H), 2.58 (q, 2H), 1.43 (s, 9H), 1.18 (t, 3H), 1.11 (t, 3H).

### Example 27

### Preparation of 2-{3,5-diethyl-4-[(5-nitro-2-pyridinyl)sulfanyl]-1H-pyrazol-1-yl}ethylamine, Trifluoroacetic Acid Salt

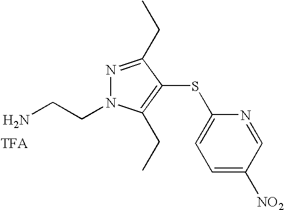

The compound was prepared by the procedure for the TFA salt of Example 7. Product (0.185 g, 89%): Mp. 166-168° C., ¹H NMR (300 MHz, CD₃OD) δ 9.12 (dd, 1H), 8.36 (dd, 1H), 7.20 (d, 1H), 4.42 (t, 1H), 3.49 (t, 2H), 2.76 (q, 2H), 2.59 (q, 2H), 1.18 (t, 3H), 1.12 (t, 3H).

### Example 28

### Preparation of tert-butyl 2-{4-[(5-amino-2-pyridinyl)sulfanyl]-3,5-diethyl-1H-pyrazol-1-yl}ethylcarbamate

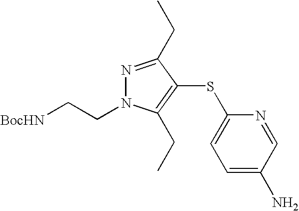

The compound was prepared by the reduction procedure described for Example 7. Product (0.65, 69%): R_{f}=0.20 (66% EtOAc in Hexane), MS (Electronspray) 392 (M+H)⁺, ¹H NMR (300 MHz, CDCl₃) δ 7.92 (d, 1H), 6.79 (dd, 1H), 6.52 (d, 1H), 5.03 (t, 1H), 4.11 (t, 2H), 3.54 (q, 2H), 2.65 (q, 2H), 2.57 (q, 2H), 1.39 (s, 9H), 1.13 (t, 3H), 1.04 (t, 3H).

### Example 29

### Preparation of tert-butyl 2-[4-({5-[(2,2-dimethylpropanoyl)amino]-2-pyridinyl}sulfanyl)-3,5-diethyl-1H-pyrazol-1-yl]ethylcarbamate (67)

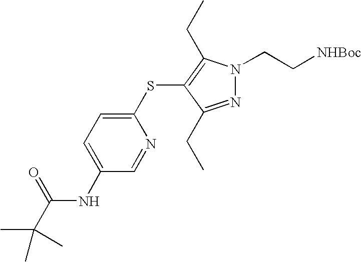

The compound was prepared by the procedure for Example 8. Product (0.185 g, 76%): R_{f}=0.66 (EtOAc), MS (Electronspray) 476 (M+H)⁺. ¹H NMR (300 MHz, CDCl₃) δ 8.37 (d, 1H), 7.97 (dd, 1H), 6.68 (d, 1H), 5.01 (t, 1H), 4.17 (t, 2H), 3.60 (q, 2H), 2.68 (q, 2H), 2.60 (q, 2H), 1.43 (s, 9H), 1.31 (s, 9H), 1.17 (t, 3H), 1.08 (t, 3H).

### Example 30

### Preparation of N-{6-[(3,5-diethyl-1-propyl-1H-pyrazol-4-yl)sulfanyl]-3-pyridinyl}-2,2 dimethylpropanamide, Trifluoroacetic Acid Salt

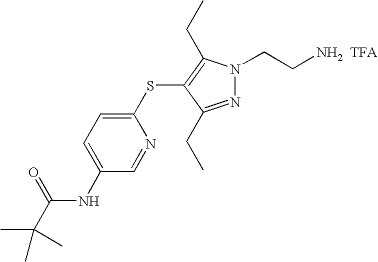

The compound was prepared by the procedure for the TFA salt of Example 7. Product (0.189 g, 100%): ¹H NMR (300 MHz, CD₃OD) δ 8.58 (d, 1H), 7.89 (dd, 1H), 7.01 (d, 1H), 4.40 (t, 2H), 3.49-3.46 (m, 2H), 2.75 (q, 2H), 2.58 (q, 2H), 1.27 (s, 9H), 1.19-1.08 (m, 6H).

### Example 31

### Preparation of 2-{4-[(4-chlorophenyl)sulfinyl]-3,5-diethyl-1H-pyrazol-1-yl}ethylamine

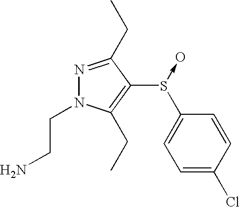

The compound was prepared by the procedure for Example 1. Product (0.146 g, 39%): MS (Electronspray) 326 (M+H)⁺, ¹H NMR (300 MHz, CDCl₃) δ 7.49-7.42 (m, 4H), 4.02 (t, 2H), 3.15 (t, 2H), 2.86-2.69 (m, 2H), 2.54-2.36 (m, 2H), 1.13-1.05 (m, 6H).

### Example 32

### Preparation of (2Z)-2-butenedioic Acid Compound with 2-{4-[(4-chlorophenyl)sulfinyl]-3,5-diethyl-1H-pyrazol-1-yl}ethanamine (1:1)

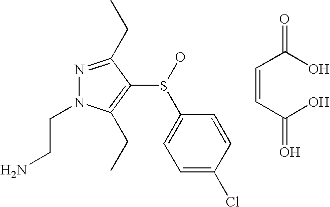

The maleic acid salt was prepared from Example 31 by the procedure for Example 5. Product (0.188 g, 95%): Mp. 159-161° C., ¹H NMR (300 MHz, CD₃OD) δ 7.61-7.54 (m, 4H), 6.25 (s, 2H), 4.35 (t, 2H), 3.45 (t, 2H), 2.97-2.77 (m, 2H), 2.52-2.35 (m, 2H), 1.18-1.06 (m, 6H).

### Example 33

### Preparation of methyl 6-[(1-{2-[(tert-butoxycarbonyl)amino]ethyl}-3,5-diethyl-1H-pyrazol-4-yl)sulfanyl]nicotinate

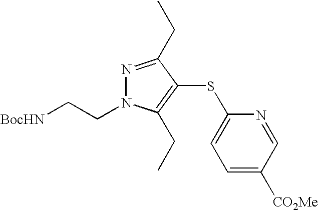

### Step 1. Preparation of methyl 6-[(5-methyl-2-pyridinyl)disulfanyl]nicotinate

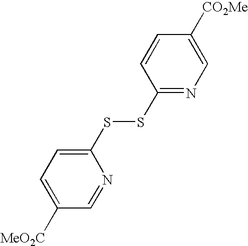

Sulfuryl chloride (0.95 mL, 11.8 mmol) was added to a suspension of 6-mercapto-nicotinic acid methyl ester (1 g, 5.91 mmol) in carbon tetrachloride (25 mL). The mixture was stirred at room temperature for 1.5 days and concentrated. The product (0.62 g, 63%) was isolated by column chromatography (50% EtOAc in Hexane). R_{f}=0.48 (50% EtOAc in Hexane), MS (Electronspray) 337 (M+H)⁺, ¹H NMR (300 MHz, CDCl₃) δ 8.97 (d, 2H), 8.27 (dd, 2H), 7.79 (d, 2H), 3.92 (s, 6H).

### Step 2. Preparation of methyl 6-[(2-oxo-1-propionylbutyl)sulfanyl]nicotinate

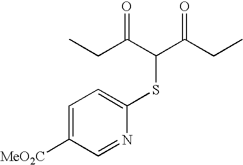

Sodium hydride (0.119 g, 2.975 mmol, 60%) was added to a solution of the product of step 1 (0.6 g 1.786 mmol) and 3,5 heptadione (0.229 g, 1.786 mmol) at room temperature. The mixture was stirred at room temperature for 16 h and quenched with water (10 mL), extracted with ethyl acetate (3×25 mL) and the organic extract dried over MgSO₄ and concentrated. The product (0.13 g, 25%) was isolated by column chromatography (33% EtOAc in Hexane). R_{f}=0.20 (33% EtOAc in Hexane), MS (Electronspray) 296 (M+H)⁺, ¹H NMR (300 MHz, CDCl₃) δ 8.95 (d, 2H), 8.06 (dd, 2H), 7.08 (d, 2H), 3.86 (s, 3H), 2.76-2.53 (m, 4H), 1.08-0.99 (m, 6H).

### Step 3. Preparation of methyl 6-[(1-{2-[(tert-butoxycarbonyl)amino]ethyl}-3,5-diethyl-1H-pyrazol-4-yl)sulfanyl]nicotinate

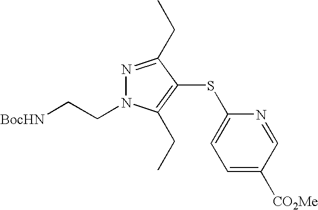

(2-Hydrazino-ethyl)-carbamic acid tert-butyl ester, prepared by reation of 2(BOC amino)ethyl bromide (Aldrich Chemical Co.) and hydrazine, (0.081 g, 0.462 mmol) was added to a solution of the product of step 1 (0.13 g, 0.44 mmol) in ethanol (2 mL) at room temperature. The mixture was stirred at room temperature for 16 h and refluxed for 24 h and concentrated. The product (0.16 g, 63%) was isolated by column chromatography (50% EtOAc in Hexane). R_{f}=0.42 (50% EtOAc in Hexane). MS (Electronspray) 335 (M+H)⁺. ¹H NMR (300 MHz, CDCl₃) δ 8.95 (d, 1H), 7.98 (dd, 1H), 6.76 (d, 1H), 5.04 (t, 1H), 4.17 (t, 2H), 3.59 (q, 2H), 2.66 (q, 2H), 2.57 (q, 2H), 1.41 (s, 9H), 1.15 (t, 3H), 1.07 (t, 3H).

### Example 34

### Preparation of (2Z)-2-butenedioic Acid Compound with methyl 6-{[1-(2-aminoethyl)-3,5-diethyl-1H-pyrazol-4-yl]sulfanyl}nicotinate (1:1)

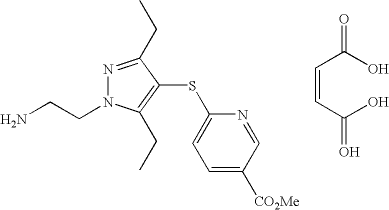

A solution of Example 33 (0.13 g, 0.44 mmol) in methanol (1 mL) was treated with HCl in dioxane (1 mL, 4M). The solution was stirred at room temperature for 16 h and concentrated. The residue was in saturated sodium bicarbonate (3 mL), extracted with ethyl acetate (2×10 mL) and the organic extract dried over MgSO₄ and concentrated. The resulting oil was dissolved in ethyl acetate (1 mL) and treated with maleic acid at room temperature. The mixture was filtered and the solid dried under vacuum to give a white solid (0.04 g). ¹H NMR (300 MHz, CDCl₃) δ 8.88 (d, 1H), 8.09 (dd, 1H), 7.03 (d, 1H), 6.21 (s, 2H), 4.65 (t, 21), 4.33 (q, 2H), 3.88 (s, 31H), 2.76 (q, 2H), 2.54 (q, 2H), 1.13 (t, 3H), 1.09 (t, 3H).

### Example 35

### Preparation of 2-{3,5-diethyl-4-[(4-fluorophenyl)sulfanyl]-1Ipyrazol-1-yl}ethylamine

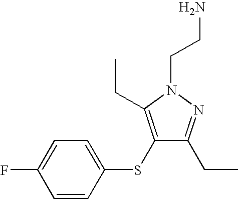

To a suspension of 3,5-diethyl-4-[(4-fluorophenyl)sulfanyl]-1H-pyrazole, prepared as in Example 1, (0.6 g, 2.4 mmol) in acetonitrile (1.5 mL) was added sodium hydroxide (0.38 g, 9.60 mmol) and the mixture was stirred under argon for 30 min at room temperature. Chloroethylamine hydrochloride (0.42 g, 3.6 mmol) and tetrabutylammonium hydrogen sulfate (0.33 g, 0.096 mmol) were added and the mixture refluxed for 3 h and diluted with ethyl acetate (20 mL). The solid was filtered and the filtrate was concentrated under reduced pressure. The residue was dissolved in ethyl acetate (20 mL), dried over Na₂SO₄ and filtered through a plug of silica gel using 5% methanol in ethyl acetate as the eluant to give 0.7 g, 100% of the product. R_{f}=0.08 (5% MeOH in EtOAc), MS (Electronspray) 294 (M+H)⁺, ¹H NMR (300 MHz, CDCl₃) δ 6.95-6.86 (m, 4H), 4.10 (t, 1H), 3.17 (t, 21), 2.71 (q, 2M), 2.59 (q, 2H), 1.16 (t, 3H), 1.08 (t, 3H).

### Example 36

### Preparation of N-(2-{3,5-diethyl-4-[(4-fluorophenyl)sulfanyl]-1H-pyrazol-1-yl}ethyl)-N-methylamine

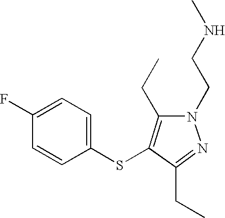

To a solution of sodium methoxide (0.24 g, 4.41 mmol) in anhydrous methanol (2.2 mL) was added a solution of Example 35 (0.59 g, 2 mmol) in methanol (2.2 mL). The resultant mixture was added to a suspension of parafomaldehyde (0.083 g, 3.75 mmol) in methanol (2.3 mL). The mixture was stirred at room temperature for 16 h. Sodium borohydride (0.076 g, 2.mmol) was added in one portion and the mixture stirred at room temperature for 1 h, quenched with I N sodium hydroxide (2 mL) and extracted with ethyl acetate (2×30 mL). The combined organic extracts were concentrated under reduced pressure. The product was isolated by column chromatography (25% MeOH in EtOAc) (0.44 g, 72%). R_{f}=0.07 (5% MeOH in EtOAc), MS (Electronspray) 308 (M+H)⁺, ¹H NMR (300 MHz; CDCl₃) δ 6.94-6.89 (m, 4H), 4.16 (t, 1H), 3.06 (t, 3H), 2.70 (q, 2H), 2.59 (q, 2H), 1.16 (t, 3H), 1.05 (t, 3H).

### Example 37

### Preparation of (2Z)-2-butenedioic Acid Compound with 2-{3,5-diethyl-4-[(4-fluorophenyl)sulfanyl]-1H-pyrazol-1-yl}-N-methylethanamine (1:1)

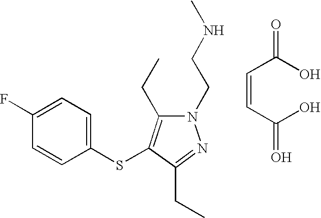

A solution of maleic acid (0.17 g, 1.46 mmol) in ether (5 mL) was added to a solution of Example 36 (0.44 g, 1.43 mmol) in ether (5 mmol). The mixture was stirred under argon for 30 min and ether removed with a syringe. The residue was washed twice with ether (10 mL) and dried under vacuum to give 0.5 g, 84% of product. MS (Electronspray) 308 (M+H)⁺, RT=3.57, Mp. 95-97° C. ¹H NMR (300 MHz, CDCl₃) δ 7.13-6.99 (m, 4H), 5.99 (s, 2H), 4.32 (t, 1H), 3.35 (t, 3H), 2.68-2.43 (m, 6H), 1.07 (t, 3H), 1.00 (t, 3H).

### Example 38

### Preparation of N-(2-{3,5-diethyl-4-[(4-fluorophenyl)sulfanyl]-1H-pyrazol-1-yl}ethyl)-N,N-dimethylamine

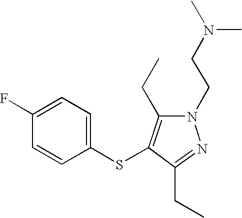

To a solution of sodium methoxide (0.24 g, 4.41 mmol) in anhydrous methanol (2.2 mL) was added a solution of Example 35 (0.59 g, 2 mmol) in methanol (2.2 mL). The resultant mixture was added to a suspension of parafomaldehyde (0.083 g, 3.75 mmol) in methanol (2.3 mL). The mixture was stirred at room temperature for 16 h. Sodium borohydride (0.076 g, 2.mmol) was added in one portion and the mixture stirred at room temperature for 1 h, quenched with I N sodium hydroxide (2 mL) and extracted with ethyl acetate (2×30 mL). The combined organic extracts were concentrated under reduced pressure. The product was isolated by column chromatography (25% MeOH in EtOAc) (0.05 g, 8%). R_{f}=0.22 (5% MeOH in EtOAc), ¹H NMR (300 MHz, CDCl₃) δ 6.93-6.89 (m, 4H), 4.15 (t, 1H), 2.78-2.57 (m, 6H), 2.30 (s, 6H), 1.15 (t, 3H), 1.09 (t, 3H).

### Example 39

### Preparation of (2Z)-2-butenedioic Acid Compound with 2-{3,5-diethyl-4-[(4-fluorophenyl)sulfanyl]-1H-pyrazol-1-yl}-N,N-dimethylethanamine (1:1)

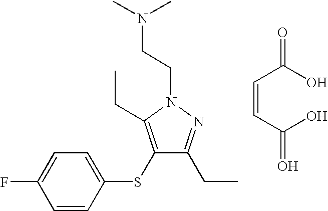

A solution of maleic acid (0.13 g, 1.12 mmol) in ether (5 mL) was added to a solution of Example 38 (0.37 g, 1.151 mmol) in ether (5 mL). The mixture was stirred under argon for 30 min and ether removed with a syringe. The residue was washed twice with ether (10 mL) and dried under vacuum to give 0.43 g, 86% of product. MS (Electronspray) 322 (M+H)⁺, RT=3.54, ¹H NMR (300 MHz, DMSO) δ 7.13-6.96 (m, 4H), 6.03 (s, 2H), 4.15 (t, 1H), 3.52 (t, 2H), 2.85 (s, 6H), 2.69 (q, 2H), 2.47 (q, 2H), 1.09-0.98 (m, 6H).

### Example 40

### Preparation of tert-butyl (3R)-3-{3,5-diethyl-4-[(4-fluorophenyl)sulfanyl]-1H-pyrazol-1-yl}-1-pyrrolidinecarboxylate

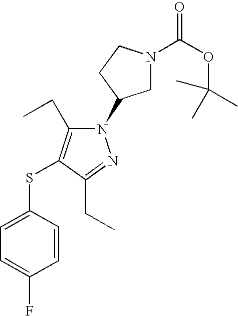

### Step 1. Preparation of tert-butyl (3S)-3-hydroxy-1-pyrrolidinecarboxylate

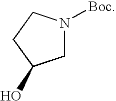

To a solution of (R)-3-pyrrolidinol (1.00 g, 11 mmol) in tetrahydrofuran (22 mL) were treated with di-tert-butyl dicarbonate (3.01 g, 14 mmol) and dichloromethane (2.5 mL) at 0 C. The reaction mixture was stirred at room temperature for 5 hours and concentrated under vacuum. The residue was diluted with ethyl acetate and quenched with saturated sodium bicarbonate. The aqueous layer was extracted with ethyl acetate (3×). The combined organics were dried over MgSO₄ and concentrated under vacuum to provide an oil (2.00 g, 100%). R_{f}=0.57 (67% hexane in ethyl acetate); MS (Electronspray) 188 (M+H)⁺, ¹H NMR (300 MHz, CDCl₃) δ 4.45-4.43 (m, 1H), 3.49-3.36 (m, 4H), 2.20-1.80 (m, 2H), 1.45 (s, 9H).

### Step 2. Preparation of tert-butyl (3S)-3-[(methylsulfonyl)oxy]-1-pyrrolidinecarboxylate

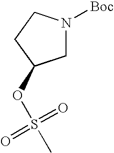

Methanesulfonyl chloride (0.72 mL, 9.24 mmol) was added to a solution of the product of step 1 (1.45 g, 7.744 mmol), dimethylamino pyridine (0.047 g) and triethylamine (1.4 mL, 10.067 mmol) in acetonitrile (13 mL). The mixture was stirred at 0° C. under argon for 30 min and quenched with water (10 mL) and extracted with ethyl acetate (3×30 mL). The combined extracts were dried over MgSO₄ and concentrated and purified by column chromatography (50% EtOAc in Hexane) to give 1.54 g of product (75%). R_{f}=0.25 (50% EtOAc in Hexane), ¹H NMR (300 MHz, CDCl₃) δ 5.28-5.23 (m, 1H), 3.72-3.43 (m, 4H), 3.04 (s, 3H), 2.27-2.04 (m, 2H), 1.49 (s, 9H).

### Step 3. Preparation of tert-butyl (3R)-3-{3,5-diethyl-4-[(4-fluorophenyl)sulfanyl]-1H-pyrazol-1-yl}-1-pyrrolidinecarboxylate

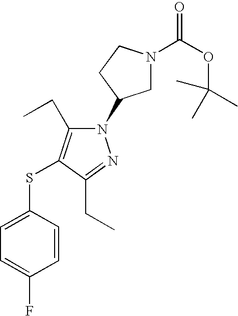

To a solution of the product of step 2 (0.17 g, 0.641 mmol) in tetrahydrofuran (4 mL) were added the pyrazole 3,5-diethyl-4-[(4-fluorophenyl)sulfanyl]-1H-pyrazole (0.107 g, 0.427 mmol) and then sodium hydride (0.026 g, 0.641 mmol) at 0° C. The reaction mixture was stirred at 0° C. for 1 h and warmed up to room temperature. Stirring was continued at room temperature for 2 h. The mixture was then refluxed for 17 h, quenched with water and extracted with ethyl acetate (3×20 mL). Combined organic extracts was dried over MgSO₄ and the residue purified by column chromatography (17% EtOAc in Hexane) to give 0.1 g, 56% of product. R_{f}=0.58 (50% EtOAc in Hexane), ¹H NMR (300 MHz, CDCl₃) δ 6.93-6.90 (m, 4H), 4.77-4.72 (m, 1H), 3.80-3.71 (m, 2H), 3.50-3.41 (m, 2H), 2.75-2.57 (m, 5H), 2.27 (m, 1H), 1.47 (s, 9H), 1.17-1.05 (m, 6H).

### Example 41

### Preparation of 3,5-diethyl-4-[(4-fluorophenyl)sulfanyl]-1-[(3R)-3-pyrrolidinyl]-1H-pyrazole, Hydrochloride

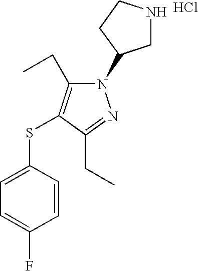

To a solution of Example 40 (0.1 g, 0.238 mmol) in ether (1 mL) was added HCl (6.4 mL, 2 M) in ether at room temperature. The mixture was stirred at room temperature for 2 days and concentrated. MS (Electronspray) 320 (M+H)⁺, ¹H NMR (300 MHz, DMSO) δ 7.12-6.95 (m, 4H), 5.21-5.16 (m, 1H), 3.63-3.30 (m, 4H), 2.70 (q, 2H), 2.50-2.12 (m, 4H), 1.08-0.97 (m, 6H).

### Example 42

### Preparation of N-(2-{3,5-diethyl-4-[(4-fluorophenyl)sulfanyl]-1H-pyrazol-1-yl}-1,1-dimethylethyl)-4-nitrobenzenesulfonamide

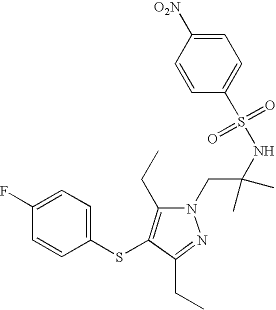

### Step 1. Preparation of N-(2-hydroxy-1,1-dimethylethyl)-4-nitrobenzenesulfonamide

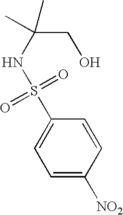

Nosyl chloride (12.43 g, 0.0561 mol) was added in portions to a cooled (0° C.) solution of 2-amino-2-methyl propanol (5 g, 0.0561 mol) and triethylamine (7.8 mL, 0.0841 mol) in dichloromethane (110 mL). The resulting cloudy yellow solution was allowed to warm up to room temperature and stirred for 1 h and quenched with water (20 mL). The organic layer was isolated and dried over MgSO₄ and concentrated. The product (9.43 g, 62%) was purified by column chromatography (50% EtOAc in Hexane). R_{f}=0.30 (50% EtOAc in Hexane), MS (Electronspray) 274 (M)⁺, ¹H NMR (300 MHz, CDCl₃) δ 8.35 (d, 2H), 8.09 (d, 2H), 5.14 (s, 1H), 3.49 (d, 2H), 2.08 (t, 1H), 1.20 (s, 6H).

### Step 2. Preparation of 2,2-dimethyl-1-[(4-nitrophenyl)sulfonyl]aziridine

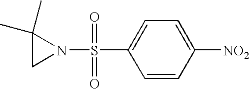

Methanesulfonyl chloride (2.67 mL, 0.0344 mol) was added to a suspension of the product of step 1 (9 g, 0.0328 mol), and triethylamine (9.15 mL, 0.0656 mol) in dichloromethane (100 mL). The mixture was stirred at room temperature for 4 h and quenched with water (30 mL). The organic layer was isolated and dried over MgSO₄ and concentrated. The product (8.32 g, 98%) was purified by column chromatography (33% EtOAc in Hexane). R_{f}=0.63 (50% EtOAc in Hexane), ¹H NMR (300 MHz, CDCl₃) δ 8.36 (d, 2H), 8.13 (d, 2H), 2.52 (s, 2H), 1.60 (s, 6H).

### Step 3. Preparation of N-(2-{3,5-diethyl-4-[(4-fluorophenyl)sulfanyl]-1H-pyrazol-1-yl}-1,1-dimethylethyl)-4-nitrobenzenesulfonamide

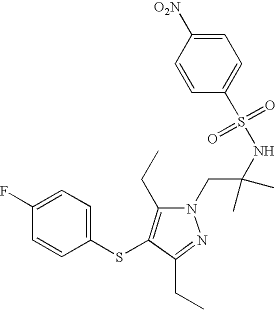

To a solution of the pyrazole 3,5-diethyl-4[(4-fluorophenyl)sulfanyl]-1H-pyrazole (0.98 g, 3.9 mmol) in tetrahydrofuran (39 mL) was added sodium hydride (0.234 g, 5.85 mmol) at room temperature. The mixture was stirred for 5 min and the product from step 2 (1 g, 3.90 mmol) was added, stirring was continued at room temperature under argon for 16 h. The reaction mixture was quenched with water and extracted with ethyl acetate (3×40 mL). Combined organic extracts were washed with brine, dried over MgSO₄ and concentrated under reduced pressure. The residue purified by column chromatography (33% EtOAc in Hexanes) to give 1.65 g, 83% of product. R=0.38, MS (Electronspray) 507 (M+H)⁺, ¹H NMR (300 MHz, CDCl₃) δ 8.31 (d, 2H), 8.08 (d, 2H), 6.91 (d, 4H), 3.90 (s, 2H), 2.6 (q, 4H), 1.28-1.22 (m, 12H).

### Example 43

### Preparation of 2-{3,5-diethyl-4-[(4-fluorophenyl)sulfanyl]-1H-pyrazol-1-yl}-1,1-dimethylethylamine

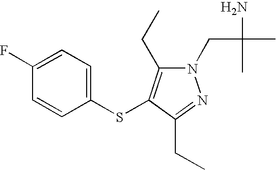

The mixture of Example 42 (1.34 g, 2.645 mmol), Benzenethiol (0.81 mL, 7.935 mmol), potassium carbonate (1.46 g, 10.58 mmol), acetonitrile (65 mL) and dimethylsulfoxide (1.32 mL) was heated at 50° C. for 2 days. Water (5 mL) was added and the mixture extracted with ethyl acetate (3×30 mL). Combined organic extracts were dried over Na₂SO₄ and concentrated under reduced pressure. The residue purified by column chromatography (25% MeOH in EtOAc) to give 0.81 g, 95% of product. MS (Electronspray) 322 (M+H)⁺, ¹H NMR (300 MHz, CDCl₃) δ 6.93-6.89 (m, 4H), 3.94 (s, 2H), 2.68 (q, 2H), 2.59 (q, 2H), 1.26-1.04 (m, 12H).

### Example 44

### Preparation of (2Z)-2-butenedioic Acid Compound with 1-{3,5-diethyl-4-[(4-fluorophenyl)sulfanyl]-1H-pyrazol-1-yl}-2-methyl-2-propanamine (1:1)

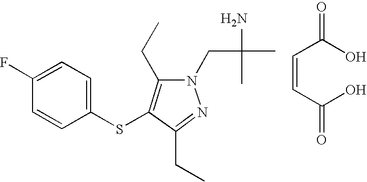

A solution of maleic acid (0.195 g, 1.68 mmol) in ether (5 mL) was added to a solution of Example 43 (0.54 g, 1.68 mmol) in ether (5 mL). The mixture was stirred under argon for 30 min and the ether was removed with a syringe. The residue was washed twice with ether (10 mL) and dried under vacuum to give 0.69 g, 93% of product. MS (Electronspray) 322 (M+H)⁺, RT=3.98, Mp. 120-122° C. ¹H NMR (300 MHz, DMSO) δ 7.14-6.98 (m, 4H), 6.00 (s, 2H), 4.20 (s, 2H), 2.70 (q, 2H), 2.50 (q, 2H), 1.22 (s, 6H), 1.09 (t, 3H), 0.99 (t, 3H).

### Example 45

### Preparation of ethyl 2-{3,5-diethyl-4-[(4-fluorophenyl)sulfanyl]-1H-pyrazol-1-yl}propanoate

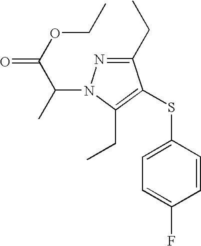

To a solution of 3,5-diethyl-4-[(4-fluorophenyl)sulfanyl]-1H-pyrazole (0.17 g, 0.641 mmol), prepared as in Example 1, in tetrahydrofuran (5 mL) were added ethyl 2-bromopropionate (0.107 g, 0.427 mmol) and then sodium hydride (0.026 g, 0.641 mmol) at room temperature. The reaction mixture was stirred at room temperature C for 30 min, quenched with water and extracted with ethyl acetate (2×20 mL). Combined organic extracts were dried over MgSO₄ and concentrated under reduced pressure. The residue purified by column chromatography (17% EtOAc in Hexane) to give 0.1 g, 56% of product. R_{f}=0.6 (50% EtOAc in Hexane). ¹H NMR (300 MHz, CDCl₃) δ 6.94-6.86 (m, 4H), 4.93 (q, 1H), 4.22-4.15 (m, 4H), 2.71-2.56 (m, 4H), 1.87 (d, 3H), 1.32-1.04 (m, 12H).

### Example 46

### Preparation of 2-{3,5-diethyl-4-[(4-fluorophenyl)sulfanyl]-1H-pyrazol-1-yl}propanamide

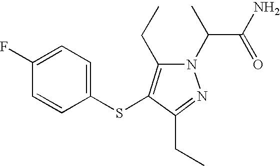

The compound prepared in Example 45 (0.43 g, 1.198 mmol) was treated with ammonia in methanol (10 mL, 2 M) at room temperature. The solution was stirred at room temperature for 2 days and concentrated to give 0.4 g of product (used in the next step without further purification). R_{f}=0.16 (50% EtOAc in Hexanes), MS (Electronspray) 322 (M+H)⁺, ¹H NMR (300 MHz, CDCl₃) δ 6.94-6.88 (m, 4H), 4.83 (q, 1H), 2.75-2.59 (m, 4H), 1.85 (d, 3H), 1.18 (t, 3H), 1.06 (t, 3H).

### Example 47

### Preparation of 2-{3,5-diethyl-4-[(4-fluorophenyl)sulfanyl]-1H-pyrazol-1-yl}propylamine

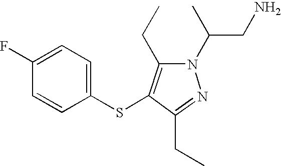

A mixture of Example 46 (0.1, 0.311 mmol) and borane-tetrahydrofuran (1.5 mL, 1M) complex in tetrahydrofuran (2 mL) was refluxed for 1 h and slowly quenched with methanol (1 mL). 6 N HCl (1 mL) was added and the mixture refluxed for 1.5 h and cooled to room temperature. The mixture was basified with 1 N sodium hydroxide and extracted with ethyl acetate (2×15 mL), dried over MgSO₄ and concentrated to give 0 1 g of product. R=0.14 (25% MeOH in EtOAc).

### Example 48

### Preparation of 2-{3,5-diethyl-4-[(4-fluorophenyl)sulfanyl]-1H-pyrazol-1-yl}propylamine

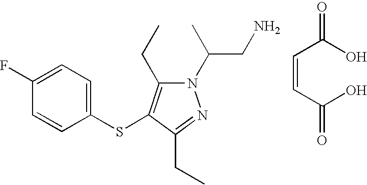

A solution of maleic acid (0.0.0332 g, 0.286 mmol) in ether (1 mL) was added to a solution of Example 47 (0.08 g, 0.26 mmol) in ether (1 mL). The mixture was stirred under argon for 1 h and filtered. The residue was washed twice with ether (10 mL) and dried under vacuum to give 0.08 g, 78% of product. MS (Electronspray) 308 (M+H)⁺, RT=3.46, mp. 162° C. ¹H NMR (300 MHz, DMSO) δ 7.12-6.99 (m, 4H), 5.99 (s, 2H), 4.60-4.54 (m, 1H), 3.39-3.18 (m, 2H), 2.71-2.43 (m, 4H), 1.37 (d, 3H), 1.07 (t, 3H), 0.99 (t, 3H).

### Example 49

### Preparation of {3,5-diethyl-4-[(4-fluorophenyl)sulfanyl]-1H-pyrazol-1-yl}acetonitrile

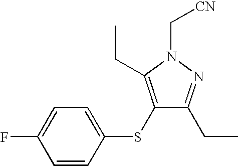

The compound was prepared by the procedure for Example 1. Product (0.55 g, 95%): R=0.53 (50% EtOAc in Hexane), GC/MS 289 (M)⁺, ¹H NMR (300 MHz, CDCl₃) δ 6.97-6.89 (m, 4H), 5.01 (s, 2H), 2.77 (q, 2H), 2.58 (q, 2H), 1.25-1.14 (m, 6H).

### Example 50

### Preparation of 3-{3,5-diethyl-4-[(4-fluorophenyl)sulfanyl]-1H-pyrazol-1-yl}-2-butanone, Dihydrochloride

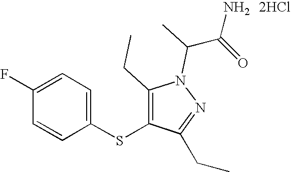

To a solution of Example 46 (0.129 g, 0.384 mmol) in ether (1 mL) was added HCl (3 mL, 2M) in ether at room temperature. The mixture was stirred at room temperature for 2 h and the solid filtered and dried under vacuum to give 0.08 g of product. mp 167° C., MS (Electronspray) 322 (M+H)⁺, ¹H NMR (300 MHz, DMSO) δ 7.08-6.97 (m, 4H), 4.92 (q, 1H), 2.68-2.41 (m, 6H), 1.61 (d, 3H), 1.03 (t, 3H), 0.94 (t, 3H).

### Example 51

### Preparation of tert-butyl 2-(4-{[4-(3,3-dimethyl-2,5-dioxo-1-pyrrolidinyl)phenyl]sulfanyl}-3,5-diethyl-1H-pyrazol-1-yl)ethylcarbamate

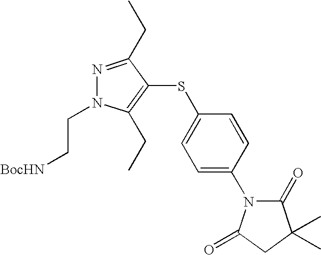

To a solution of the aniline prepared in Example 7 (0.4 g, 1.02 mmol) and triethylamine (0.06 mL, 0.41 mmol) in pyridine (5 mL) and toluene (5 mL) was added 2,2-dimethylsuccinic anhydride (0.2 g, 1.56 mmol). The mixture was refluxed under argon overnight and concentrated under reduced pressure. The product was isolated by column chromatography (33% EtOAc in Hexane) (0.41 g, 80%). MS (Electronspray) 501 (M+H)⁺, ¹H NMR (300 MHz, CDCl₃) δ 7.12 (d, 2H), 7.03 (d, 2H), 5.00 (t, 1H), 4.17 (t, 2H), 3.61 (t, 2H), 2.70-2.54 (m, 6H), 1.43 (s, 9H), 1.41 (s, 6H), 1.18 (t, 3H), 1.09 (t, 3H).

### Example 52

### Preparation of 1-(4-{[1-(2-aminoethyl)-3,5-diethyl-1I-pyrazol-4-yl]sulfanyl}phenyl)-3,3-dimethyl-2,5-pyrrolidinedione

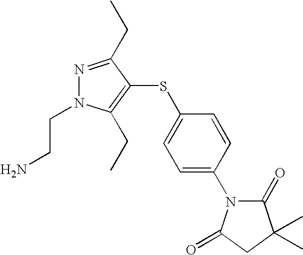

The HCl salt was prepared by the procedure described for step 5, Example 1. Product (0.39 g, 100%): mp 167° C. ¹H NMR (300 MHz, DMSO) δ 6.06 (d, 2H), 5.98 (d, 2H), 3.31 (t, 2H), 2.38 (t, 2H), 1.69 (q, 2H), 1.64 (s, 2H), 1.51 (q, 2H), 0.27 (s, 6H), 0.08 (t, 3H), 0.02 (t, 3H).

### Example 53

### Preparation of tert-butyl 2-(4-{[4-(3,5-dioxo-4-morpholinyl)phenyl]sulfanyl}-3,5-diethyl-1H-pyrazol-1-yl)ethylcarbamate

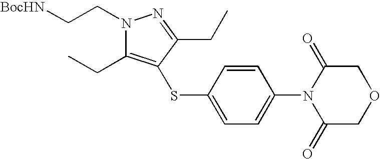

The compound was prepared by the procedure for Example 51. Product (0.11 g, 22%). MS (Electronspray) 521 (M+H)⁺, ¹H NMR (300 MHz, CDCl₃) δ 7.05 (d, 2H), 6.98 (d, 2H), 5.02 (t, 1H), 4.50 (s, 4H), 4.21 (t, 2H), 3.61 (t, 2H), 2.72-2.58 (in, 4H), 1.43 (s, 3H), 1.20 (s, 6H), 1.11 (t, 3H).

### Example 54

### Preparation of 4-(4-{[1-(2-aminoethyl)-3,5-diethyl-1H-pyrazol-4-yl]sulfanyl}phenyl)-3,5-morpholinedione hydrochloride

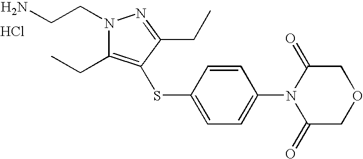

The HCl salt was prepared by the procedure described for step 5, Example 1. Product (0.08 g, 94%): Mp. 190° C. ¹H NMR (300 MHz, DMSO) δ

### Example 55

### Preparation of tert-butyl 2-{3,5-dimethyl-4-[(4-nitrophenyl)sulfanyl]-1H-pyrazol-1-yl}ethylcarbamate

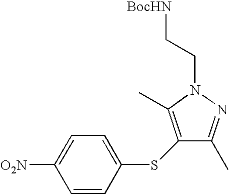

The compound was prepared by the procedure described for Example 1. R_{f}=0.50 (50% EtOAc in Hexane), MS (Electronspray) 393 (M+H)⁺, ¹H NMR (300 MHz, CDCl₃) δ 8.23 (d, 2H), 7.12 (d, 2H), 3.65 (d, 2H), 2.35 (s, 3H), 2.30 (s, 3H), 1.50 (s, 9H).

### Example 56

### Preparation of tert-butyl 2-{4-[(4-aminophenyl)sulfanyl]-3,5-dimethyl-1H-pyrazol-1-yl}ethylcarbamate

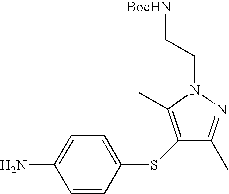

The compound was prepared by the procedure described for Example 6.

### Example 57

### Preparation of 2-{3,5-dimethyl-4-[(4-nitrophenyl)sulfanyl]-1I-pyrazol-1-yl}ethylamine

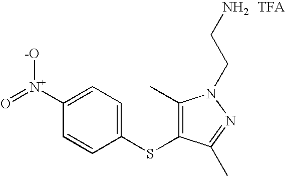

The compound was prepared by the procedure for the TFA salt of Example 7. R_{f}=0.3 (50% EtOAc in Hexanes), MS (Electronspray) 293 (M+H)⁺, RT=3.13. ¹H NMR (300 MHz, CDCl₃) δ 8.02 (d, 2H), 7.05 (d, 2H), 4.37 (m, 2H), 3.85 (t, 2H), 2.23 (s, 3H), 2.19 (s, 3H).

### Example 58

### Preparation of 4-{[1-(2-aminoethyl)-3,5-dimethyl-1H-pyrazol-4-yl]sulfanyl}aniline, Trifluoroacetic Acid Salt

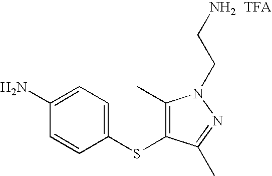

The compound was prepared by the procedure for the TFA salt of Example 7. R_{f}=0.15 (20% MeOH in CH₂Cl₂), MS (Electronspray) 263 (M+H)⁺, RT=2.47, ¹H NMR (300 MHz, CDCl₃) δ 8.09 (d, 2H), 7.10 (d, 2H), 4.20 (m, 2H), 3.20 (t, 2H), 2.23 (s, 3H), 2.00 (s, 3H).

### Example 59

### Preparation of tert-butyl 2-[4-({4-[(2,2-dimethylpropanoyl)amino]phenyl}sulfanyl)-3,5-dimethyl-1H-pyrazol-1-yl]ethylcarbamate

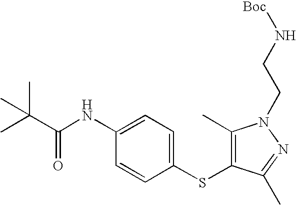

To solution of Example 56 (0.45 g, 1.24 mmol) in dichloromethane (5 mL) was added poly-4-vinyl-pyridine (0.409 g, 3.72 mmol) followed by trimethylacetyl chloride (0.153 mL, 1.24 mmol), the reaction mixture stirred 18 hours at room temperature. The reaction mixture was filtered through a coarse filter frit and the filtrate was concentrated to produce a yellow oil that was chromatographed using 30% ethyl acetate in hexane to afford a yellow solid (0.27 g, 49%). MS (Electronspray) 447 (M+H)⁺. R_{f}=0.5 (50% EtOAc/hexane)¹H NMR (300 MHz, CDCl₃) δ 7.39 (d, 2H), 6.95 (d, 2H), 4.18 (m, 2H), 3.53 (m, 2H), 2.21 (s, 3H), 2.18 (s, 3H), 1.64 (s, 9H), 1.35 (s, 9H).

### Example 60

### Preparation of N-(4-{[1-(2-aminoethyl)-3,5-dimethyl-1H-pyrazol-4-yl]sulfanyl}phenyl)-2,2-dimethylpropanamide, Trifluoroacetic Acid Salt

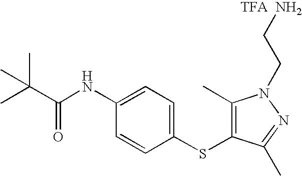

The compound was prepared by the procedure for the TFA salt of Example 7. R_{f}=0.30 (20% MeOH in CH₂Cl₂), MS (Electronspray) 347 (M+H)⁺, RT=3.15. ¹H NMR (300 MHz, CDCl₃) δ 7.36 (d, 2H), 7.00 (d, 2H), 4.67 (m, 2H), 3.62 (m, 2H), 2.39 (s, 3H), 2.25 (s, 3H), 1.64 (s, 9H).

### Example 61

### Preparation of tert-butyl 2-{4-[(4-{[(ethylamino)carbonothioyl]amino}phenyl)sulfanyl]-3,5-dimethyl-1H-pyrazol-1-yl}ethylcarbamate

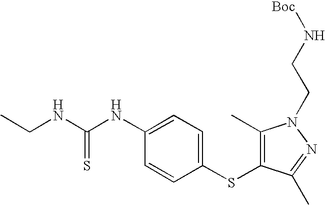

The compound was prepared by the procedure described for Example 8. Product (0.02 g, 54%), R_{f}=0.30 (20% MeOH in CH₂Cl₂), MS (Electronspray) 450 (M+H)⁺.

### Example 62

### Preparation of N-(4-{[1-(2-aminoethyl)-3,5-dimethyl-1H-pyrazol-4-yl}sulfanyl}phenyl)-N′-ethylthiourea, Trifluoroacetic Acid Salt

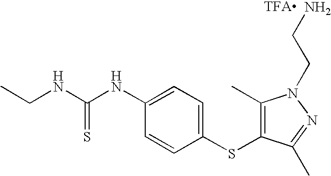

The compound was prepared by the procedure described for Example 7. R=0.3 (20% MeOH in CH₂Cl₂), MS (Electronspray) 350 (M+H)⁺, RT=2.69, ¹H NMR (300 MHz, CDCl₃) 7.05 (d, 2H), 7.00 (d, 2H), 4.50 (t, 2H), 2.64 (t, 2H), 2.64 (q, 2H), 2.36 (s, 3H), 2.20 (s, 3H), 1.20 (t, 3H).

### Example 63

### Preparation of tert-butyl 2-{3,5-dimethyl-4-[(4-nitrophenyl)sulfanyl]-1H-pyrazol-1-yl}ethyl(methyl)carbamate

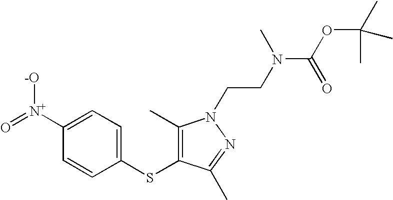

The compound was prepared by the procedure described for Example 22. R_{f}=0.45 (50% EtOAc in Hexane), MS (Electronspray) 406 (M+H)⁺, ¹H NMR (300 MHz, CDCl₃) δ 8.07 (d, 2H), 7.05 (d, 2H), 4.23 (m, 2H), 3.64 (t, 2H), 2.7 (d, 3H), 2.23 (s, 3H), 2.19 (s, 3H), 1.44 (s, 9H).

### Example 64

### Preparation of tert-butyl 2-{4-[(4-aminophenyl)sulfanyl]-3,5-dimethyl-1H-pyrazol-1-yl}ethyl(methyl)carbamate

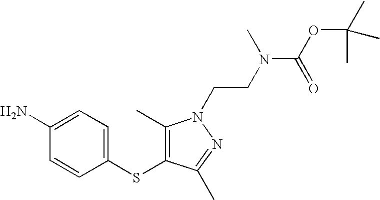

The compound was prepared by the procedure for the TFA salt of Example 7. R_{f}=0.35 (50% EtOAc in Hexane), MS (Electronspray) 377 (M+H)⁺.

### Example 65

### Preparation of tert-butyl 2-[4-({4-[(2,2-dimethylpropanoyl)amino]phenyl}sulfanyl)-3,5-dimethyl-1H-pyrazol-1-yl]ethyl(methyl)carbamate

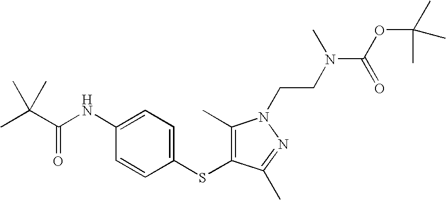

The compound was prepared by the procedure described for Example 8. R_{f}=0.30 (50% EtOAc in Hexane), MS (Electronspray) 461 (M+H)⁺. ¹H NMR (300 MHz, CDCl₃) δ 7.39 (d, 2H), 6.97 (d, 2H), 4.20 (m, 2H), 3.60 (t, 2H), 2.8 (s, 3H), 2.23 (s, 3H), 2.20 (s, 3H), 1.45 (s, 9H), 1.34 (s, 9H).

### Example 66

### Preparation of N-[4-({3,5-dimethyl-1-[2-(methylamino)ethyl]-1H-pyrazol-4-yl}sulfanyl)phenyl]-2,2-dimethylpropanamide, Trifluoroacetic Acid Salt

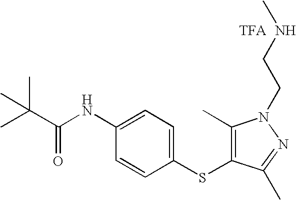

The compound was prepared by the procedure for the TFA salt of Example 7. MS (Electronspray) 361 (M+H)⁺. ¹H NMR (300 MHz, CDCl₃) δ 7.37 (d, 2H), 3.97 (d, 2H), 4.43 (t, 2H), 3.55 (t, 2H), 2.88 (s, 3H), 2.29 (s, 3H), 2.19 (s, 3H), 1.30 (s, 9H).

### Example 67

### Preparation of tert-butyl 2-{4-[(4-bromophenyl)sulfanyl]-3,5-diethyl-1H-pyrazol-1-yl}ethylcarbamate

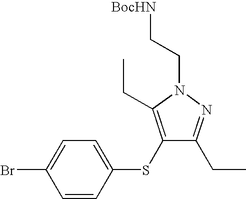

The compound was prepared by the procedure described for Example 14. MS (Electronspray) 455 (M+2)⁺. ¹H NMR (300 MHz, CDCl₃) δ 7.28 (d, 2H), 6.78 (d, 2H), 4.92 (s, 1H), 4.12 (t, 2H), 3.56 (q, 2H), 2.65 (q, 2H), 2.51 (q, 2H), 1.41 (s, 9H), 1.14 (t, 3H), 1.03 (t, 3H).

### Example 68

### Preparation of tert-butyl 2-[4-({4-[(1E)-3,3-dimethyl-1-butenyl]phenyl}sulfanyl)-3,5-diethyl-1H-pyrazol-1-yl]ethylcarbamate

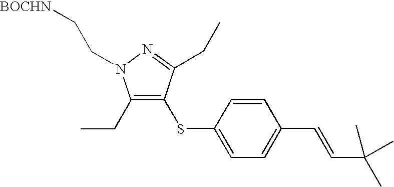

### Step 1. Preparation of 2-[(1E)-3,3-dimethyl-1-butenyl]-1,3,2-benzodioxaborole

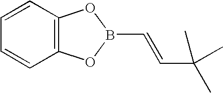

Catechol borane (2.6 mL, 24.34 mmol) and 3,3-dimethyl-1-bytyne were combined under argon at 5° C. in a flask. The flask was the sealed and solution was stirred for 30 minutes at room temperature and 2 hours at 70° C. The solution was then cooled to room temperature and concentrated under reduced pressure to afford 2-[(1E)-3,3-dimethyl-1-butenyl]-1,3,2-benzodioxaborole (4.1 g, 700%), GC/MS 203 (M+H). ¹H NMR (300 MHz, CDCl₃) δ 7.20 (m, 2H), 7.05 (m, 2H), 5.74 (s, 1H), 5.68 (s, 1H), 1.11 (s, 9H).

### Step 2. Preparation of tert-butyl 2-[4-({4-[(1E)-3,3-dimethyl-1-butenyl]phenyl}sulfanyl)-3,5-diethyl-1H-pyrazol-1-yl]ethylcarbamate

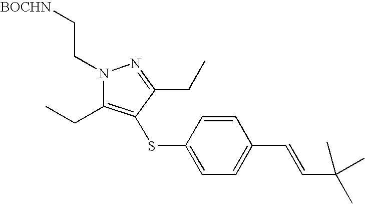

The compound of Example 67 (0.1 g, 0.22 mmol) was dissolved in N,N-dimethylformamide (1.5 mL) and degassed for 15 minutes. Degassing was continued during the addition of 2-[(1E)-3,3-dimethyl-1-butenyl]-1,3,2-benzodioxaborole (step 1, 0.067 g, 0.33 mmol), saturated sodium carbonate (0.22 mL, 0.44 mmol), palladium acetate (5 mg, 0.022 mmol) and tri-o-tolylphosphine (0.0134 g, 0.044 mmol). The mixture was then heated to reflux for 5 hours and then cooled to room temperature and extract with ethyl acetate. Combined organic were then dried over anhydrous sodium sulfate and concentrate under reduced pressure. The resulting residue was purified with flash chromatography (Biotage flash 40M ) using 50% hexane in ethyl acetate to afford product (51 mg, 51%). MS (Electronspray) 458 (M+H)+¹H NMR (300 MHz, CDCl₃) δ 7.17 (d, 1H), 6.85 (m, 3H), 6.17 (d, 2H, 4.95 (br s, H), 4.15 (t, 2H), 3.57 (q, 2H), 2.57 (m, 4H), 1.43 (s, 9H), 1.16 (t, 3H), 1.08 (s, 9H), 1.04 (t, 3H).

### Example 69

### Preparation of 2-[4-({4-[(1E)-3,3-dimethyl-1-butenyl]phenyl}sulfanyl)-3,5-diethyl-1H-pyrazol-1-yl]ethylamine, Trifluoroacetic Acid Salt

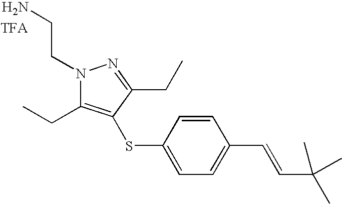

To a solution of the compound of Example 68 (50 mg, 0.11 mmol) in dichloromethane (1.8 mL) was added trifluoroacetic acid (0.5 mL). The resulting solution was stirred at room temperature for 4 hours and then concentrated under reduced pressure. Residue was purified with reversed phase HPLC to give the product (18 mg, 35%). MS (Electronspray) 358 (M+H)⁺, HPLC, RT=3.54. ¹H NMR (300 MHz, CDCl₃) δ 7.19 (d, 2H), 6.89 (d, 2H), 6.19 (s, 2H), 4.35 (s, 2H), 3.56 (m, 2H), 2.65 (q, 2H), 2.53 (q, 2H), 1.65 (s, 2H), 1.09 (s, 9H), 1.04 (m, 6H).

### Example 70

### Preparation of 2-tert-butyl 2-(3,5-diethyl-4-{[4-(1,3-thiazol-2-yl)phenyl]sulfanyl}-1H-pyrazol-1-yl)ethylcarbamate

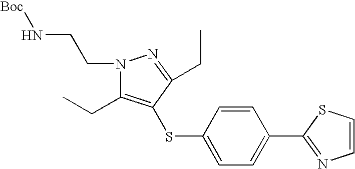

The product of Example 67 (100 mg, 0.22 mmol) was dissolved in N,N-dimethylformamide (1.5 mL) and degassed for 15 minutes. Degassing was continued during the addition of 2-(trimethylstannyl)-1,3-thiazole (82 mg, 0.33 mmol) and Tetrakis (triphenylphosphine) palladium (0) (25 mg, 0.022 mmol). Mixture was then heated to reflux for 18 hours and then cooled to room temperature and extract with ethyl acetate. Combined organic extracts were then dried over anhydrous sodium sulfate and concentrate under reduced pressure. The resulting residue was purified with reversed phase HPLC to afford the product (22 mg, 30%). MS (Electronspray) 459 (M+H)⁺. ¹H NMR (300 MHz, CDCl₃) δ 8.80 (s, 1H), 8.02 (s, 1H), 7.39 (d, 2H), 7.00 (d, 2H), 5.13 (s, 1H), 4.23 (s, 2H), 3.59 (s, 2H), 2.63 (m, 4H), 1.42 (s, 9H), 1.11 (m, 6H).

### Example 71

### Preparation of 2-(3,5-diethyl-4-{[4-(1,3-thiazol-2-yl)phenyl]sulfanyl}-1H-pyrazol-1-yl)ethylamine, Trifluoroacetic Acid Salt

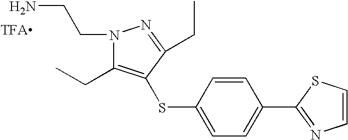

To a solution of Example 70 (20 mg, 0.044 mmol) in dichloromethane (0.75 mL) was added trifluoroacetic acid (0.1 mL). The resulting solution was stirred at room temperature for 3 hours and then concentrated under reduced pressure to afford product (16.5 mg, 80%). MS (Electronspray) 359 (M+H)⁺, HPLC RT=2.86. ¹H NMR (300 MHz, CDCl₃) δ 8.84 (s, 1H), 7.98 (s, 1H), 7.35-7.37 (m, 3H), 6.94 (d, 2H), 4.31 (s, 2H), 3.48 (s, 2H), 2.65 (q, 2H), 2.52 (q, 2H), 1.01-1.15 (m, 6H).

### General Methods of Combinatorial Chemistry Syntheses

Reactions were carried out in 8-mL glass vials with Teflon-lined screw caps, or in a polypropylene reaction block consisting of a 6×8 matrix of forty-eight 5.6-mL reaction wells, with each reaction well incorporating a 15-45 micron polyethylene frit; reaction blocks of this type are commercially available as FlexChem™ reactor blocks from Robbins Scientific Corporation, Sunnyvale, Calif. The reactor blocks are sealed with rubber gaskets and a clamping device, and can be heated with mixing by rotation in an oven (Robbins Scientific). The following are specific examples of the above method.

### Example 72

### Step 1. Preparation of N-alkylated pyrazoles

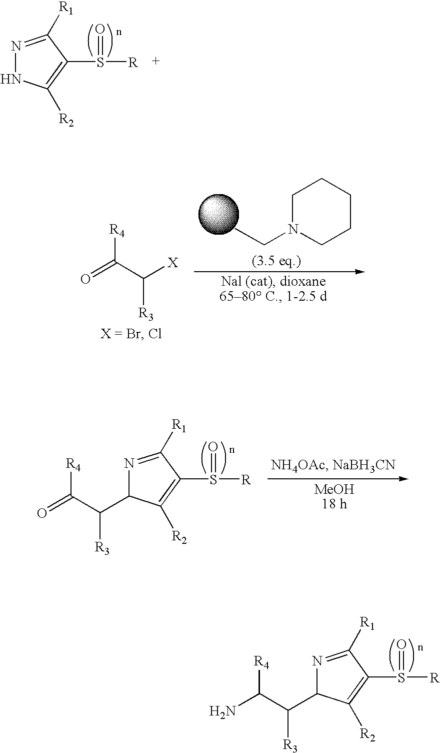

In a typical procedure, solutions of α-bromomethyl and/or x-chloromethyl ketones were prepared as 1.0 M in dioxane, and solutions of pyrazoles (commercially-available or prepared as described as in Example 1, steps 3 and 4) were prepared as 250 mM in dioxane. To each reaction well in a polypropylene reaction block was added sodium iodide (5 mg), followed by piperidinomethyl polystyrene (200 mg, 0.7 mmol, 3.5 mmol/g), a solution of the desired pyrazole (800 μL, 0.2 mmol), and a solution of the desired α-halomethyl ketone (600 μL, 0.6 mmol). The reaction block was sealed with rubber gaskets and clamped, then heated at 65-80° C. for 1-2.5 days, with mixing by rotation. After allowing the reaction block to cool to room temperature, the block was disassembled, and the reaction well contents were filtered into a collection 96-well deep-well microtiter plate, washing with acetonitrile or dichloromethane. The filtrate solutions were analyzed for purity and correct identity by HPLC/UV/ELSD and LC/MS, and were evaporated to dryness using a multiple sample centrifugal vacuum evaporator.

Step 2. Reductive Amination of N-Acylated pyrazoles with Ammonium Acetate and Sodium Cyanoborohydride

Crude products from the previous step (0.2 mmol scale) were dissolved in 400 μL methanol, and 50 μL of this solution (ca. 15-25 μmol) was added to each reaction well, as desired. Solutions of ammonium acetate in methanol (1.5 M) and sodium cyanoborohydride in methanol (1.0 M) were prepared. To each reaction well was added powdered 4 Å molecular sieves (25 mg), followed by the ammonium acetate solution (167 μL, 250 μmol), and then the reaction mixture was mixed by orbital shaking for 5-10 min. Sodium cyanoborohydride (25 μL, 25 μmol) solution was then added to each reaction well, and then the reaction block was sealed with a gasket and rotated at room temperature for 18 h. The reaction block was disassembled and the reaction contents reaction well contents were filtered into a collection 96-well deep-well plate, washing with 2×250 mL dichloromethane. Using a well-ventilated fume hood, 6.0 N HCl (20 μL) was added to each filtrate solution, followed by 5.0 NaOH (120 mL) and water (500 EL). After the reaction mixture was mixed for 1 h, the organic phase was removed to a clean vial or a to a well in a 96-well deep-well microtiter plate. The product solutions were analyzed for purity and correct identity by HPLC/UV/ELSD and LC/MS, and were evaporated to dryness using a multiple sample centrifugal vacuum evaporator. For compounds of particular interest, this step was carried out on four-fold scale, and the product was purified by preparative reverse phase HPLC, and characterized by LC/MS and NMR.

### Example 73

### Preparation of ethyl 1-{2-[(tert-butoxycarbonyl)amino]ethyl}-4-[(4-fluorophenyl)sulfanyl]-3-methyl-1H-pyrazole-5-carboxylate and ethyl 1-{2-[(tert-butoxycarbonyl)amino]ethyl}-4-[(4-fluorophenyl)sulfanyl]-5-methyl-1H-pyrazole-3-carboxylate

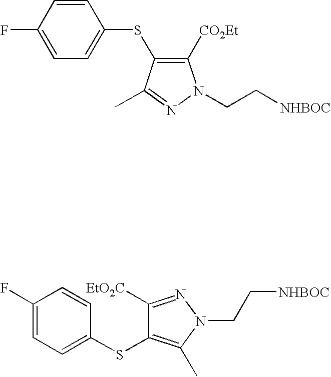

### Step 1. Preparation of ethyl 3-chloro-2,4-dioxopentanoate

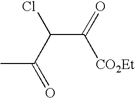

A solution of thionyl chloride (5.72 mL, 71.2 mmol) in toluene (20 mL) was added to a solution of ethyl 2,4-diovalerate (11.3 gm, 71.2 mmol) in toluene (70 mL). The resulting mixture was stirred at rt for ˜65 h and was then concentrated under reduced pressure to afford crude product (13 gm) which was used in the next step without further purification: MS (Electronspray) 193.1 (M+H)⁺.

### Step 2. Preparation of ethyl 3-[(4-fluorophenyl)sulfanyl]-2,4-dioxopentanoate

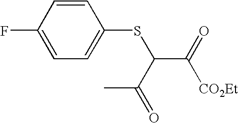

Caution: exothermic reaction. Pyridine (2.3 mL, 28.6 mmol) was added very slowly to a mixture of the compound prepared in Step 1 (5 gm, 26.0 mmol) and 4-fluorobenzenethiol (2.77 mL, 26.0 mmol), giving a dark solution which was stirred for 30 min. at rt. Ethyl acetate was added and the organic layer was washed with water and 1N HCl (2×). The organic layer was dried and concentrated to give crude product (6.79 gm) which was used in the next step without further purification: MS (Negative ion electronspray) 283.3 (M−H)—; ¹H NMR (300 MHz, CDCl₃) δ 7.25-7.15 (m, 2H), 7.05-6.95 (m, 2H), 4.28 (q, 2H), 2.34 (s, 3H), 1.21 (t, 3H).

### Step 3. Preparation of ethyl 4-[(4-fluorophenyl)sulfanyl]-3-methyl-1H-pyrazole-5-carboxylate

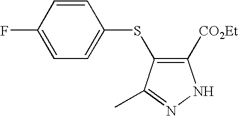

Hydrazine hydrate (1.1 mL, 22.7 mmol) was added to a solution of the product of Step 2 (6.79 gm, 23.9 mmol) in ethanol (95 mL). The mixture was stirred at rt for 1.5 h and concentrated. Purification of the crude material by flash chromatography (Biotage Flash 40, 4:2 hexane:ethyl acetate) afforded product (4.8 g, 72%): MS (Electronspray) 280.9 (M+H)⁺; ¹H NMR (300 MHz, DMSO-d₆) δ 7.10-6.90 (m, 4H), 4.08 (q, 2H), 2.17 (s, 3H), 1.08 (t, 3H).

Step 4. Preparation of ethyl 1-{2-[(tert-butoxycarbonyl)amino]ethyl}-4-[(4-fluorophenyl)sulfanyl]-3-methyl-1H-pyrazole-5-carboxylate

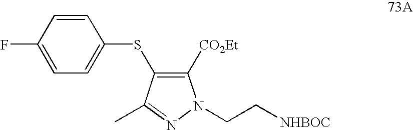

### and

### ethyl 1-{2-[(tert-butoxycarbonyl)amino]ethyl}-4-[(4-fluorophenyl)sulfanyl]-5-methyl-1H-pyrazole-3-carboxylate

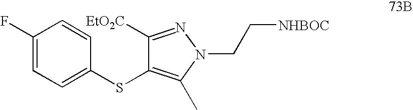

Cesium carbonate (2.97 gm, 9.14 mmol) was added to a solution of the product prepared in Step 3 (1.28 gm, 4.57 mmol) in dimethylformamide (30 mL) and the mixture was stirred for 5 min. tert-Butyl 2-bromoethylcarbamate (1.23 gA, 5.48 mmol) was added and the reaction mixture was stirred for 4 h. Water and ethyl acetate were added and the layers were separated. The aqueous layer was extracted with ethyl acetate (3×). The combined extracts were dried and concentrated. Purification by flash chromatography (Biotage Flash 40, 4:2 hexane:ethyl acetate) afforded the products: 73A (476 mg, 25%) MS (Electronspray) 423.9 (M+H)⁺; and 73B (214 mg, 11%): MS (Electronspray) 423.9 (M+H)⁺.

### Example 74

### Preparation of ethyl 1-(2-aminoethyl)-4-[(4-fluorophenyl)sulfanyl]-3-methyl-1H-pyrazole-5-carboxylate, Trifluoroacetic Acid Salt

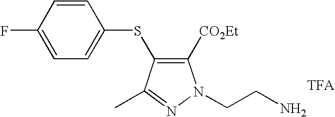

Neat TFA (0.1 mL, 1.18 mmol) was added to a solution of Example 73A (100 mg, 0.221 mmol) in dichloromethane (1.6 mL). The resulting mixture was stirred for 65 h at rt and was then concentrated under reduce pressure. The resulting mixture was then dissolved in a minimum amount of dichloromethane. Diethyl ether was added, and filtration provided product (80 mg, 83%) as a white solid: MS (Electronspray) 324.1 (M+H)⁺; ¹H NMR (300 MHz, DMSO-d₆) δ 7.92 (br, s, 3H), 7.20-7.10 (m, 4H), 4.63 (t, 2H), 4.23 (q, 2H), 3.40-3.20 (m, 2H), 2.11 (s, 3H), 1.13 (t, 3H).

### Example 75

### Preparation of ethyl 1-(2-aminoethyl)-4-[(4-fluorophenyl)sulfanyl]-5-methyl-1H-pyrazole-3-carboxylate, Trifluoroacetic Acid Salt

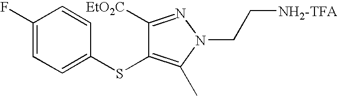

The product was prepared using a procedure similar to that above starting from Example 73B. Yield: 55 mg white solid, 52%. MS (Electronspray) 324.0 (M+H)⁺; ¹H NMR (300 MHz, DMSO-d₆) δ 7.93 (br, s, 3H), 7.11-7.05 (m, 4H), 4.40 (t, 2H), 4.16 (q, 2H), 3.40-3.20 (m, 2H), 2.32 (s, 3H), 1.15 (t, 31).

### Example 76

### Preparation of tert-butyl 2-[4-[(4-fluorophenyl)sulfanyl]-5-(hydroxymethyl)-3-methyl-1H-pyrazol-yl]ethylcarbamate

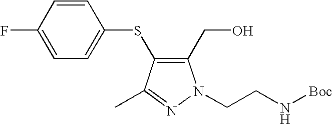

To a 0° C. solution of Example 73A (500 mg, 1.18 mmol) in dry tetrahydrofuran (1 mL) was added a solution of lithium aluminum hydride (1M, 3.54 mL) in tetrahydrofuran. The mixture was stirred for 30 min. and was then warmed to rt over 2 h. Celite® (1 gm) was added followed by slow addition of water (1 mL), sodium hydroxide (2N, 1 mL), and again water (3 mL). The suspension was stirred for 1 h and then filtered. The filtrate was concentrated to afford product (410 mg, 91%): MS (Electronspray) 381.9 (M+H)⁺; ¹H NMR (300 MHz, DMSO) δ 7.15-6.85 (m, 4H), 5.33 (t, 1H), 4.47 (d, 2H), 4.18 (t, 2H), 3.35-3.25 (m, 2H), 2.03 (s, 3H), 1.33 (t, 9H)

### Example 77

### Preparation of {1-(2-aminoethyl)-4-[(4-fluorophenyl)sulfanyl]-3-methyl-1H-pyrazol-5-yl}methanol

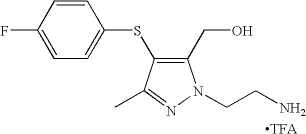

The compound was prepared using a procedure similar to that for Example 74. Yield: 47 mg, 45%. MS (Electronspray) 282.0 (M+H)⁺; ¹H NMR (300 MHz, DMSO) δ 7.90 (br s, 2H), 7.15-7.00 (m, 4H), 5.50 (br, s, 1H), 4.53 (br, s, 2H), 4.37 (t, 2H), 3.30-3.20 (m, 2H), 2.06 (s, 3H).

### Example 78

### Preparation of tert-butyl 2-[4-[(4-fluorophenyl)sulfanyl]-3-(hydroxymethyl)-5-methyl-1H-pyrazol-1-yl]ethylcarbamate

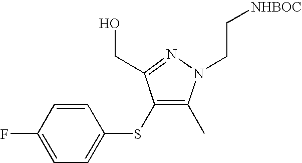

The product was prepared using a procedure similar to Example 76 starting from Example 73B. MS (Electronspray) 381.9 (M+H)⁺.

### Example 79

### Preparation of {4-[(4-fluorophenyl)sulfanyl]-5-methyl-1-propyl-1H-pyrazol-3-yl}methanol, Trifluoroacetic Acid Salt

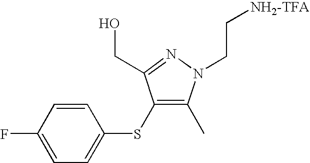

The product was prepared using a procedure similar to that for Example 74. MS (Electronspray) 282.0 (M+H)⁺.

### Example 80

### Preparation of tert-butyl 2-[4-[(4-fluorophenyl)sulfanyl]-5-(methoxymethyl)-3-methyl-1H-pyrazol-1-yl]ethylcarbamate

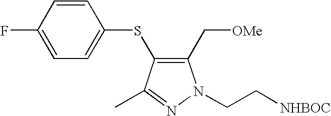

Cesium carbonate (162 mg, 0.5 mmol) was added to a solution of Example 76 in DMF (2.5 mL). Methyl iodide (0.16 mL, 2.5 mmol0 was added and the mixture was stirred at rt for 6 h. Water (5 mL), ethyl acetate (15 mL) and HCl (1N, 3 mL) were added and the layers were separated. The organic layer was dried (sodium sulfate) and concentrated. Purification of the crude material by flash chromatography (silica gel, 4:2 hexane:ethyl acetate) afforded product (60 mg, 61%): MS (Electronspray) 395.9 (M+H)⁺; ¹H NMR (300 MHz, DMSO-d₆) δ 7.10-6.90 (m, 4H), 4.41 (br, s, 1H), 4.12 (t, 2H), 3.3 (s, 3H, overlapping signal), 3.30-3.20 (m, 2H), 3.17 (s, 2H), 2.05 (s, 3H), 1.33 (t, 9H).

### Example 81

### Preparation of 4-[(4-fluorophenyl)sulfanyl]-5-(methoxymethyl)-3-methyl-1-propyl-1H-pyrazole, Trifluoroacetic Acid Salt

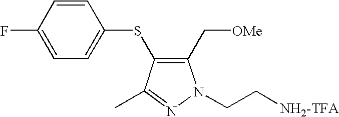

The compound was prepared using a procedure similar to that for Example 74. Yield: 60 mg, 98%. MS (Electronspray) 296.0 (M+H)⁺; ¹H NMR (300 MHz, DMSO-d₆) δ 7.84 (br, s, 3H), 7.20-7.10 (m, 4H), 4.48 (s, 2H), 4.33 (t, 2H), 3.25 (t, 2H), 3.20 (s, 3H), 2.10 (s, 3H).

### Example 82

### Preparation of tert-butyl 4-[4-[(4-fluorophenyl)sulfanyl]-3-methyl-5-(propoxymethyl)-1H-pyrazol-1-yl]butanoate

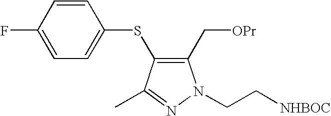

The product was prepared using a procedure similar to that of Example 80.

### Example 83

### Preparation of 2-[4-[(4-fluorophenyl)sulfanyl]-3-methyl-5-(propoxymethyl)-1H-pyrazol-1-yl]ethylamine, Trifluoroacetic Acid Salt

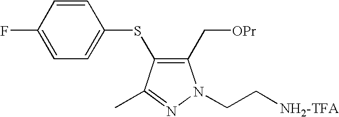

The product was prepared using a procedure similar to that of Example 74.

### Example 84

### Preparation of tert-butyl 2-{5-[(4-fluorophenoxy)methyl]-4-[(4-fluorophenyl)sulfanyl]-3-methyl-1H-pyrazol-1-yl}ethylcarbamate

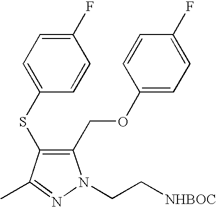

To a 0° C. solution of 4-flurophenol (48 mg, 0.43 mmol), Example 76 (180 mg, 0.472 mmol) and triphenylphosphine (113 mg, 0.43 mmol) was added diethyl azodicarboxylate (68 uL, 0.43 mmol). The mixture was allowed to warm to rt over 3 h. The mixture was concentrated and purified by flash chromatography (Biotage Flash 40, 4:2 hexane:ethyl acetate) to give product (150 mg, 73%): MS (Electronspray) 476.0 (M+H)⁺; ¹H NMR (300 MHz, DMSO) δ 7.15-6.90 (m, 8H), 5.07 (s, 2H), 4.19 (t, 2H), 3.40-3.20 (m 2H), 2.07 (s, 3H), 1.32 (s, 9H).

### Example 85

### Preparation of 2-{5-[(4-fluorophenoxy)methyl]-4-[(4-fluorophenyl)sulfanyl]-3-methyl-1H-pyrazol-1-yl}ethylamine, Trifluoroacetic Acid Salt

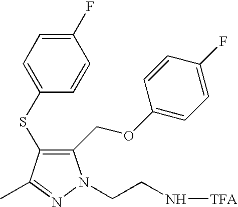

The product was prepared using a procedure similar to that of Example 74. Yield: 160 mg, quantitative. MS (Electronspray) 376.0 (M+H)⁺; ¹H NMR (300 MHz, DMSO-d₆) δ 7.90 (br, s, 3H), 7.20-6.95 (m, 8H), 5.14 (s, 2H), 4.40 (t, 2H), 3.40-3.20 (m, 2H), 2.11 (s, 3H).

### Example 86

### Preparation of tert-butyl 2-{5-(bromomethyl)-4-[(4-fluorophenyl)sulfanyl]-3-methyl-1H-pyrazol-1-yl}ethylcarbamate

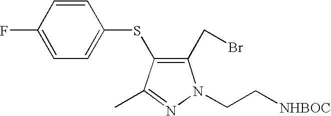

Pyridine (0.2 mL) was added to a −5° C. solution of Example 76 (1.26 mg, 3.3 mmol) in dichloromethane (25 mL). A solution of phosphorous tribromide (893 mg, 3.3 mmol) and pyridine (0.1 mL) was added dropwise and the mixture was allowed to warm to rt over 24 h. Dichloromethane and water were added and the layers were separated. The organic layer was dried (sodium sulfate) and concentrated. Purification by flash chromatography (Biotage Flash 40, 1:2 ethyl acetate:hexane) afforded product (1.2 g, 82%) as a white solid: MS (Electronspray) 443.8, 445.8 (M+H)⁺; ¹H NMR (300 MHz, DMSO-d₆) δ 7.20-6.90 (m, 4H), 4.69 (s, 2H), 4.16 (t, 2H), 3.40-3.30 (m, 2H), 2.05 (s, 3H), 1.34 (s, 9H).

### Example 87

### Preparation of tert-butyl 2-{3-(bromomethyl)-4-[(4-fluorophenyl)sulfanyl]-5-methyl-1H-pyrazol-1-yl}ethylcarbamate

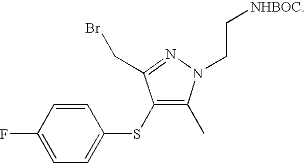

The product was prepared using a procedure similar to that of Example 86. MS (Electronspray) 443.9, 445.8 (M+H).

### Example 88

### Preparation of 2-{5-(bromomethyl)-4-[(4-fluorophenyl)sulfanyl]-3-methyl-1H-pyrazol-1-yl}ethylamine, Trifluoroacetic Acid Salt

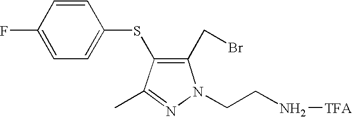

The product was prepared using a procedure similar to that of Example 74. Yield: 50 mg, 99%). MS (Electronspray) 344.0, 345.9 (M+H)⁺; ¹H NMR (300 MHz, DMSO-d₆) δ 7.91 (br, s, 3H), 7.15-7.05 (m, 4H), 4.76 (s, 2H), 3.40-3.20 (m, 2H), 2.08 (s, 3H).

### Example 89

### Preparation of 2-{3-(bromomethyl)-4-[(4-fluorophenyl)sulfanyl]-5-methyl-1H-pyrazol-1-yl}ethylamine, Trifluoroacetic Acid Salt

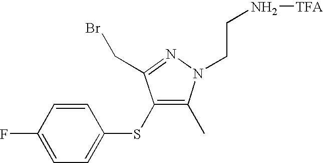

The product was prepared using a procedure similar to that of Example 74. MS (Electronspray) 344.0 (M+H)⁺, 346.0.

### Example 90

### Preparation of tert-butyl 2-{5-(fluoromethyl)-4-[(4-fluorophenyl)sulfanyl]-3-methyl-1H-pyrazol-1-yl}ethylcarbamate

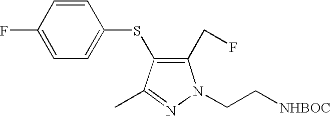

To a 0° C. solution of Example 76 (200 mg, 0.52 mmol) in dichloromethane (6 mL) was added (diethylamine)sulfur trifluoride (100 mg, 0.624 mmol). After stirring for 2 h, water (3 mL) and dichloromethane (10 mL) were added and the layers separated. The organic layer was dried (sodium sulfate) and concentrated. Purification of the crude material by flash chromatography (Biotage Flash 40, 4:2 hexane:ethyl acetate) afforded product (40 mg, 20%): MS (Electronspray) 383.9 (M+H)⁺; ¹H NMR (300 MHz, DMSO-d₆) δ 7.20-6.90 (m, 4H), 5.44 (d, 2H), 4.90 (t, 2H), 3.35-3.25 (m, 2H), 2.06 (s, 3H), 1.32 (s, 9H).

### Example 91

### Preparation of 2-{5-(fluoromethyl)-4-[(4-fluorophenyl)sulfanyl]-3-methyl-1H-pyrazol-1-yl}ethylamine, Trifluoroacetic Acid Salt

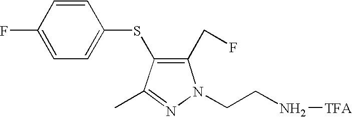

The product was prepared using a procedure similar to that of Example 74. Yield: quantitative. MS (Electronspray) 283.9 (M+H)⁺; ¹H NMR (300 MHz, DMSO-d₆) δ 7.89 (br, s, 3H), 7.20-7.05 (m, 4H), 5.52 (d, 2H), 4.41 (t, 2H), 3.30-3.20 (m, 2H), 2.11 (s, 3H).

### Example 92

### Preparation of 1-{2-[(tert-butoxycarbonyl)amino]ethyl}-4-[(4-fluorophenyl)sulfanyl]-3-methyl-1H-pyrazole-5-carboxylic Acid

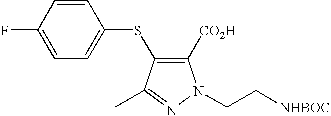

Lithium hydroxide (1N, 5 mL) was added to a solution of Example 73A (430 mg, 1.0 mmol) in dimethoxyethane (15 mL). After stirring for 1 h, the mixture was concentrated. The crude material was washed with dichloromethane (1×). Aqueous citric acid was added and the product was extracted with ethyl acetate (2×). The extracts were dried (sodium sulfate) and concentrated to afford product (400 mg, quantitative): MS (Electronspray) 395.8 (M+H)⁺; ¹H NMR (300 MHz, DMSO-d₆) δ 7.15-7.00 (m, 4H), 4.43 (t, 2H), 3.40-3.20 (m, 2H), 2.03 (s, 3H), 1.30 (s, 9H).

### Example 93

### Preparation of 1-{2-[(tert-butoxycarbonyl)amino]ethyl}-4-[(4-fluorophenyl)sulfanyl]-5-methyl-1H-pyrazole-3-carboxylic Acid

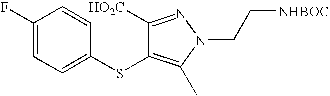

The product was prepared using a procedure similar to that above for Example 92. MS (Electronspray) 395.8 (M+H)⁺. ¹H NMR (300 MHz, DMSO-d₆) δ: 6.95-7.05 (m's, 4H), 4.18 (t, 2H), 3.2-3.4 (m, 2H), 2.22 (s, 3H), 1.29 (s, 9H).

### Example 94

### Preparation of tert-butyl 2-{5-[(cyclopropylamino)carbonyl]-4-[(4-fluorophenyl)sulfanyl-3-methyl-1H-pyrazol-1-yl}carbamate

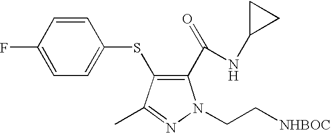

Cyclopropyl amine (51 uL, 0.75 mmol) was added at rt to a solution of Example 92 (100 mg, 0.25 mmol) and dichloromethane (3.5 mL), followed by the addition of HATU (114 mg, 0.30 mmol). The reaction mixture was concentrated and the crude material was dissolved in ethyl acetate and washed with HCl (0.5 N, 2×). The organic layer was dried and concentrated. Purification by flash chromatography (silica gel, 5:5 hexane:ethyl acetate) afforded product (60 mg, 55%): MS (Electronspray) 434.9 (M+H)⁺; ¹H NMR (300 MHz, CDCl₃) δ 7.89 (br, s, 1H), 7.05-6.90 (m, 4H), 5.15 (br, s, 1H), 4.74 (t, 2H), 3.55-3.65 (m, 2H), 2.75-2.85 (m, 1H), 2.21 (s, 3H), 1.39 (s, 9H), 0.75-0.85 (m, 2H), 0.40-0.43 (m, 2H).

### Example 95

### Preparation of 1-(2-aminoethyl)-N-cyclopropyl-4-[(4-fluorophenyl)sulfanyl]-3-methyl-1H-pyrazole-5-carboxamide, Trifluoroacetic Acid Salt

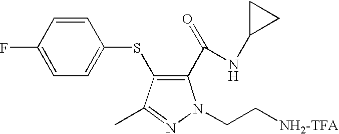

Trifluoroacetic acid (1.0 mL) was added to a solution of Example 94 (55 mg, 0.13 mmol) in dichloromethane (3 mL). The mixture was stirred at rt for 1.5 h and partially concentrated. Diethyl ether was added to the mixture causing precipitation of product. The crystals were collected by filtration and dried under reduced pressure to afford product (48 mg, 82%). Other amides that were prepared using a similar protocol were purified by reverse chromatography. Example 95: MS (Electronspray) 335.1 (M+H)⁺; ¹H NMR (300 MHz, CDCl₃) δ 8.55 (d, 1H), 7.91 (br, s, 1H), 7.20-7.00 (m, 4H), 4.40 (t, 2H), 3.26 (t, 2H) 2.70-2.81 (m, 1H), 2.15 (s, 3H), 0.66-0.73 (m, 2H), 0.41-0.47 (m, 2H).

### Example 96

### Preparation of tert-butyl 2-{4-[(3-aminophenyl)sulfanyl]-3,5-diethyl-1H-pyrazol-1-yl}ethylcarbamate

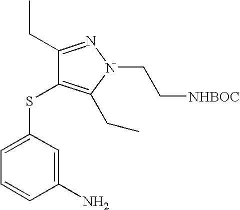

The compound was prepared by the same method described for Example 67 (400 mg, 83%): MS (Electronspray) 390.9 (M+H)⁺.

### Example 97

### Preparation of tert-butyl 2-[4-({3-[(2,2-dimethylpropanoyl)amino]phenyl}sulfanyl)-3,5-diethyl-1H-pyrazol-1-yl ethylcarbamate

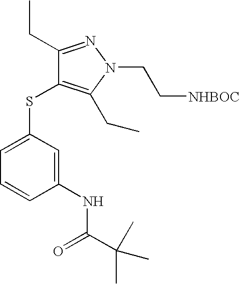

2,2-Dimethylpropanoyl chloride (34 mg, 0.28 mmol) was added to a solution of Example 96 (100 mg, 0.256 mmol) in dichloromethane (3 mL) and pyridine (0.062 mL, 0.256 mmol). The mixture was stirred for 20 min. and was then concentrated. Purification of the crude material by flash chromatography (Biotage Flash 12, 5:5 hexane:ethyl acetate) afforded product (80 mg, 66%): MS (Electronspray) 474.9 (M+H)⁺.

### Example 98

### Preparation of N-(3-{[1-(2-aminoethyl)-3,5-diethyl-1H-pyrazol-4-yl]sulfanyl}phenyl)-2,2-dimethylpropanamide, Trifluoroacetic Acid Salt

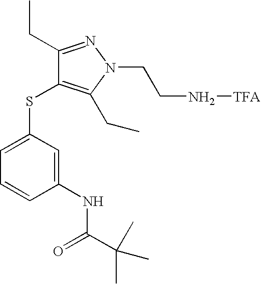

The compound was prepared using a procedure similar to that of Example 74. Yield: 38 mg, 52%. MS (Electronspray) 375.4 (M+H)⁺.

### Example 99

### Preparation of tert-butyl 2-[3,5-diethyl-4-({3-[(methylsulfonyl)amino]phenyl}sulfanyl)-1H-pyrazol-1-yl]ethylcarbamate

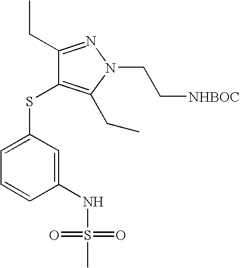

Pyridine (0.062 mL, 0.256 mmol) was added to a solution of Example 96 (100 mg, 0.256 mmol) in dichloromethane (3 mL), followed by methanesulfonyl chloride (24 uL, 0.31 mmol). After stirring at rt. for 3 h, the mixture was concentrated. Purification of the crude material by flash chromatography (Biotage Flash 12, 8:1.8:0.2 hexane:ethyl acetate:methanol) afforded product (70 mg, 58%): MS (Electronspray) 468.8 (M+H)⁺.

### Example 100

### Preparation of N-(3-{[1-(2-aminoethyl)-3,5-diethyl-1H-pyrazol-4-yl]sulfanyl}phenyl)methanesulfonamide, Trifluoroacetic Acid Salt

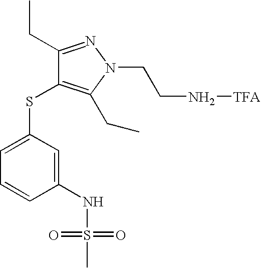

A solution of Example 99 (65 mg, 0.14 mmol) and trifluoroacetic acid (1 mL) in dichloromethane (3 mL) was stirred at rt for 2 h. Concentration of the reaction mixture followed by flash chromatography of the crude material (silica gel, 8:92 methanol:dichloromethane) afforded product (60 mg, 89%): MS (Electronspray) 369.0 (M+H)⁺.

### Example 101

### Preparation of tert-butyl 2-{4-[(4-fluorophenyl)sulfanyl]-5-hydroxy-3-methyl-1H-pyrazol-1-yl}ethylcarbamate

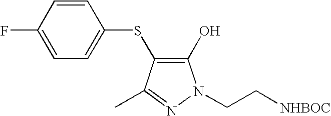

### Step 1. Preparation of ethyl 2-[(4-fluorophenyl)sulfanyl]-3-oxobutanoate

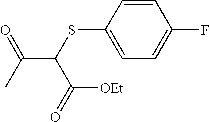

Pyridine (1.25 mL, 15.4 mmol) was added dropwise over 10 min. to a mixture of 4-fluorobenzenethiol (1.54 mL, 14 mmol) and ethyl 2-chloro-3-oxobutanoate (2.4 gm, 14 mmol). After 30 min. ethyl acetate and water were added. The layers were separated and the organic layer was washed with HCL (1N, 2×) and brine. The organic layers were dried (sodium sulfate) and concentrated to afford product (3.3 g, 92%) as a yellow oil: MS (Negative ion Electronspray) 255.3 (M−H)⁻.

### Step 2. Preparation of tert-butyl 2-{4-[(4-fluorophenyl)sulfanyl]-5-hydroxy-3-methyl-1H-pyrazol-1-yl}ethylcarbamate

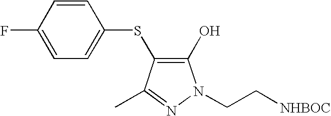

(N-Boc-2-hydrazidoethylamine) (2 gm, 11.41 mmol) was added to a solution of the product of step 1 (2 gm, 7.8 mol) in ethanol (35 mL). The mixture was refluxed, cooled to 0° C. and filtered. The collected solid was washed with ethanol and dried under reduced pressure to give product (2.05 g, 72%) as a white fluffy solid: MS (Electronspray) 367.9 (M+H)⁺; ¹H NMR (300 MHz, DMSO-d₆) δ 7.10-6.80 (m, 4H), 3.90-3.70 (m, 2H), 3.20-3.10 (m, 2H), 1.90 (br, s, 3H), 1.29 (s, 9H).

### Example 102

### Preparation of tert-butyl 2-{4-[(4-fluorophenyl)sulfanyl]-5-methoxy-3-methyl-1H-pyrazol-1-yl}ethylcarbamate

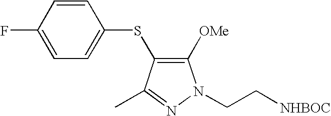

Cesium carbonate (222 mg, 0.68 mmol) was added to a solution of Example 101 (100 mg, 0.272 mmol), followed by methyl iodide (0.17 mL, 2.72 mmol). The mixture was stirred for 20 h. Water and ethyl acetate were added and the layers were separated. The aqueous layer was extracted with ethyl acetate and the combined organic layers were dried (sodium sulfate) and concentrated. Purification of the crude material by flash chromatography (Biotage 40, 4:2 hexane:ethyl acetate) afforded product (50 mg, 48%): MS (Electronspray) 381.9 (M+H)⁺; ¹H NMR (300 MHz, DMSO-d₆) δ 7.10-6.95 (m, 4H), 6.90-6.80 (m, 1H), 3.90 (s, 3H), 3.81 (t, 2H), 3.20-3.10 (m, 2H), 1.88 (s, 3H), 1.33 (s, 9H).

### Example 103

### Preparation of 1-(2-aminoethyl)-4-[(4-fluorophenyl)sulfanyl]-3-methyl-1H-pyrazol-5-ol, Trifluoroacetic Acid Salt

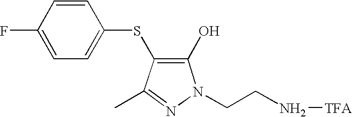

TFA (1 mL) was added to a suspension of Example 102 (70 mg, 0.19 mmol) in dichloromethane (2 mL), giving a solution which was stirred at rt for 1.5 h. The mixture was concentrated and then filtered through a short plug of silica gel (elution with 1% then 2% methanol/dichloromethane). Concentration of the filtrate afforded product (51 mg, 70%): MS (Electronspray) 268.1 (M+H)⁺; ¹H NMR (300 MHz, DMSO-d₆) δ 7.85 (br, s, 3E1), 7.10-6.90 (m, 4H), 4.00 (t, 2H), 3.35 (br, s, 1H), 3.10-3.00 (m, 2H), 1.95 (s, 3H).

### Example 104

### Preparation of 2-{4-[(4-fluorophenyl)sulfanyl]-5-methoxy-3-methyl-1H-pyrazol-1-yl}ethylamine, Trifluoroacetic Acid Salt

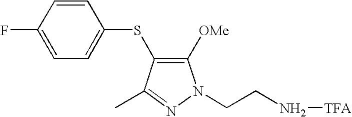

The product was prepared using a procedure similar to that of compound Example 74. Yield: 50 mg, 97%. MS (Electronspray) 282.1 (M+H)⁺; ¹H NMR (300 MHz, DMSO-d₆) δ 7.87 (br, s, 3H), 7.20-7.05 (m, 4H), 4.11 (t, 21), 4.04 (s, 3H), 3.25-3.10 (m, 2H), 2.02 (s, 3H).

### Example 105

### Preparation of 2-[5-tert-butyl-4-(4-fluorobenzyl)-3-methyl-1H-pyrazol-1-yl]ethylamine, Trifluoro-Acetic Acid Salt (105A) and 2-[3-tert-butyl-4-(4-fluorobenzyl)-5-methyl-1H-pyrazol-1-yl]ethylamine, Trifluoroacetic Acid Salt (105B)

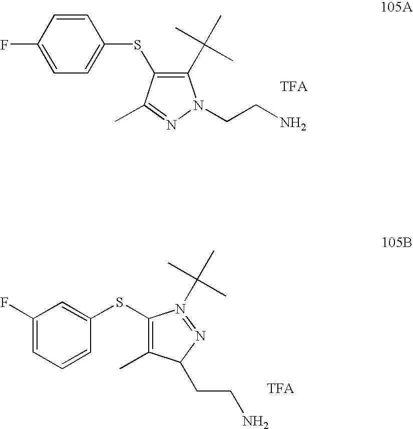

### Step 1. Preparation of 3-chloro-5,5-dimethyl-2,4-hexanedione

The compound was prepared using a procedure similar to that described in step 1, Example 73. ¹H NMR (300 MHz, CDCl₃) δ 5.09 (s, 1H), 2.38 (s 3H), 1.23 (s, 9H).

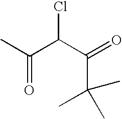

Step 2. Preparation of 3-[(4-fluorophenyl)sulfanyl]-5,5-dimethyl-2,4-hexanedione The compound was prepared using a procedure similar to that described in step 2, Example 73.

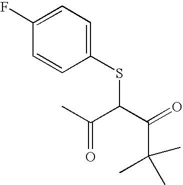

### Step 3. Preparation of tert-butyl 2-{3-tert-butyl-4-[(4-fluorophenyl)sulfanyl]-5-methyl-1H-pyrazol-1-yl}ethylcarbamate and tert-butyl 2-{5-tert-butyl-4-[(4-fluorophenyl)sulfanyl]-3-methyl-1H-pyrazol-1-yl}ethylcarbamate

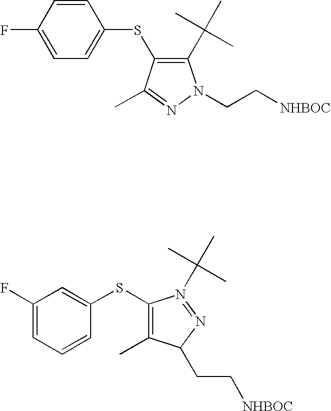

A solution of compound prepared in step 2 (1.89 g, 7.03 mmol) and BOC HEA (2.46 g, 14.1 mL) in ethanol (35 mL) was refluxed for 16 h. The reaction mixture was concentrated under reduce pressure and then dissolved in ethyl ether and ethyl acetate. The solution was washed with HCl (0.5 N) and dried (sodium sulfate). Concentration and purification of the crude material by flash chromatography (Biotage Flash 40, 1:4 ethyl acetate:hexane) provided 105A and 105B as a 2.4:1 mixture (1.29 g, 45%).

Example 105A: Ms (electron spray) 308.1 (M+H)⁺; ¹H NMR (CDCl₃, 300 MHz) δ: 1.42 (s, 9H), 2.00 (s, 3H), 3.29 (t, 2H), 4.47 (t, 2H), 6.89-6.95 (m, 2H), 7.06-7.12 (m, 2H), 7.92 (br s, 3H).

Example 105B: Ms (electron spray) 308.1 (M+H)⁺; ¹H NMR (CDCl₃, 300 MHz) δ: 1.27 (s, 9H), 2.18 (s, 3H), 3.25 (t, 2H), 4.26 (t, 2H), 6.93-6.98 (m, 2H), 7.06-7.12 (m, 2H), 7.92 (br s, 3H).

### Step 4. 2-[5-tert-butyl-4-(4-fluorobenzyl)-3-methyl-1H-pyrazol-1-yl]ethylamine, Trifluoro-Acetic Acid Salt (105A) and 2-[3-tert-butyl-4-(4-fluorobenzyl)-5-methyl-1H-pyrazol-1-yl]ethylamine, Trifluoroacetic Acid Salt (105B)

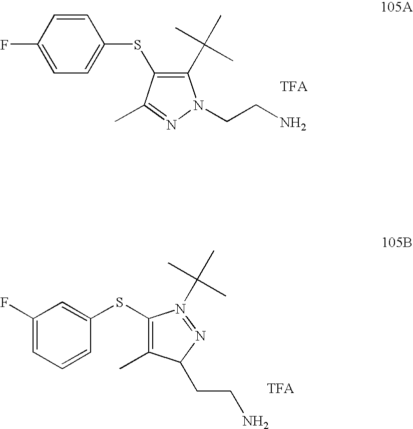

Trifluoroacetic acid (4 mL) was added to a solution of 105A and 105B (1.15 gm, 2.82 mmol) in dichloromethane (25 mL). The resulting mixture was stirred for ˜3 h at room temperature and was then concentrated under reduced pressure. Purification of the crude mixture (reverse phase, acetonitrile/water/trifluoroacetic acid) afforded 105A (135 mg, 11%) and 105B (353 mg, 30%).

Example 105A: Ms (electron spray) 308.1 (M+H)⁺; ¹H NMR (CDCl₃, 300 MHz) δ: 1.42 (s, 9H), 2.00 (s, 3H), 3.29 (t, 2H), 4.47 (t, 2H), 6.89-6.95 (m, 2H), 7.06-7.12 (m, 2H), 7.92 (br s, 3H).

Example 105B: Ms (electron spray) 308.1 (M+H)⁺; ¹H NMR (CDCl₃, 300 MHz) δ: 1.27 (s, 9H), 2.18 (s, 3H), 3.25 (t, 2H), 4.26 (t, 2H), 6.93-6.98 (m, 2H), 7.06-7.12 (m, 2H), 7.92 (br s, 3H).

### Example 106

### Preparation of tert-butyl 2-{4-[(2-ethylbutyl)sulfanyl]-3,5-dimethyl-1H-pyrazol-1-yl}ethylcarbamate

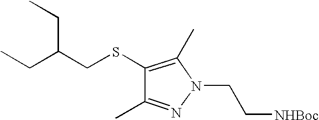

### Step 1. Preparation of S-(1-acetyl-2-oxopropyl) ethanethioate

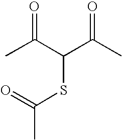

Thioacetic acid (0.37 mL, 4.2 mmol) was stirred in Et2O (50 mL) and cooled to 0° C. To this solution was added triethylamine (0.50 mL, 4.2 mmol) in one portion followed by 3-chloro-2,4-pentanedione (0.50 mL, 4.2 mmol) dropwise to produce a thick slurry. The reaction was allowed to come to rt and then filtered through silica. The silica was washed with Et2O and the combined filtrate concentrated to a yellow liquid (763 mg, 100%) which was used without further purification. R_{f}=0.63 (4:1 hex:EtOAc (v/v)); GC-MS m/z=173 (M−H); ¹H NMR (DMSO-d₆) δ 2.17 (s, 6H), 2.41 (s, 3H) ppm. {¹H}¹³C NMR (DMSO-d₆) δ 24.1, 29.5, 99.5, 194.1, 196.6 ppm.

### Step 2. Preparation of S-(1-{2-[(tert-butoxycarbonyl)amino]ethyl}-3,5-dimethyl-1H-pyrazol-4-yl) ethanethioate

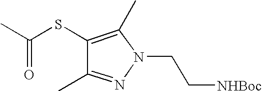

To a solution of the product of step 1 (95 mg, 0.54 mmol) in EtOH (1 mL) was added N-Boc-2-hydrazidoethylamine (200 mg, 0.10 mmol) in EtOH (1 mL). The resulting mixture was heated to reflux for 5 h, cooled to rt, and partitioned between EtOAc and water. The organic layer was collected and the aqueous layer extracted with EtOAc. The combined organics were dried (MgSO₄) and concentrated to a crude oil which was purified on silica using 2:1 EtOAc:hex as eluant, to yield, after concentration a clear oil (128 mg, 76%). R_{f}=0.70 (EtOAc); ESLC-MS m/z=314 (MH⁺); ¹H NMR (DMSO-d₆) δ 1.34 (s, 9H), 2.00 (s, 3H), 2.11 (s, 3H), 2.34 (s, 3H), 3.16-3.25 (m, 2H), 3.97-4.06 (m, 2H), 6.88-6.96 (m, 1H) ppm.

### Step 3. Preparation of tert-butyl 2-{4-[(2-ethylbutyl)sulfanyl]-3,5 dimethyl-1H-pyrazol-1-yl}ethylcarbamate

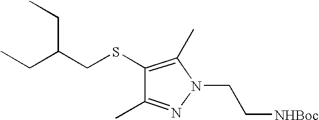

A small vial was charged with the product of step 2 (40 mg, 0.12 mmol) and TBF (1 mL). A 2.0 M solution of LiBH₄ in THF (0.10 mL, 0.20 mmol) was added and the vial was heated to 60° C. with shaking. After 3 h, the vial was cooled and I-bromo-2-ethylbutane (100 μL) was added. The vial was then heated to 70° C. for 18 h, cooled to rt and the reaction quenched by the addition of MeOH. The reaction mixture was concentrated and purified by HPLC to yield a clear oil (42 mg, 99%). R_{f}=0.36 (1:1 hex:EtOAc (v/v)); ESLC-MS m/z=356 (MH⁺); ¹H NMR (DMSO-d₆) δ 0.78 (t, J=7.4 Hz, 6H), 1.14-1.46 (m, 14H), 2.12 (s, 3H), 2.21 (s, 3H), 2.42 (d, J=6.2 Hz, 2H), 3.13-3.22 (m, 2H), 3.92-4.02 (m, 2H), 6.82-6.89 (m, 1H).

### Example 107

### Preparation of 2-{4-[(2-ethylbutyl)sulfanyl]-3,5-dimethyl-1H-pyrazol-1-yl}ethylamine, Trifluoroacetic Acid Salt

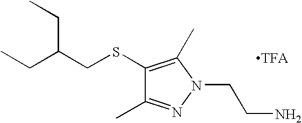

Example 106 (42 mg, 0.11 mmol) was stirred in CH₂Cl₂ (1 mL). TFA (1 mL) was added and the resulting solution was stirred for 2 h, then concentrated to a clear oil (29 mg, 66%). ESLC-MS m/z=256 (MH⁺); ¹H NMR (DMSO-d₆) δ 0.79 (t, J=7.3 Hz, 6H), 1.16-1.46 (m, 5H), 2.16 (s, 31), 2.26 (s, 3H), 2.45 (d, J=6.2 Hz, 2H), 3.13-3.22 (m, 2H), 4.13-4.20 (m, 2H), 7.86 (s, 3H) ppm.

### Example 108

### Preparation of tert-butyl 2-[3,5-diethyl-4-(propylsulfanyl)-1H-pyrazol-1-yl]ethylcarbamate

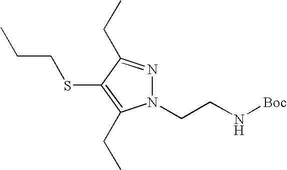

### Step 1. Preparation of S-(2-oxo-1-propionylbutyl) ethanethioate

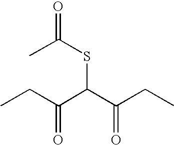

Prepared by the same method as in step 1, Example 106. The product was obtained as a yellow oil (1.52 g, 94%): R_{f}=0.46 (hexanes/ethyl acetate=7/1); ES-MS m/z 203 ((M+H)⁺);

¹H NMR (d₆-DMSO) δ 0.98 (t, J=7.3 Hz, 6H), 2.39 (s, 3H), 2.47-2.54 (m, 4H).

### Step 2. Preparation of tert-butyl 2-{4-[(1-{2-[(tert-butoxycarbonyl)amino]ethyl}-3,5-diethyl-1H-pyrazol-4-yl)disulfanyl]-3,5-diethyl-1H-pyrazol-1-yl}ethylcarbamate

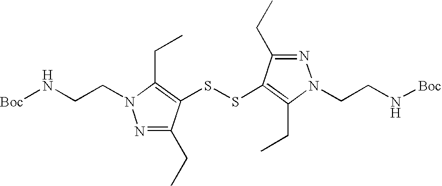

To a solution of the product prepared in step 1 (7.5 g, 37 mmol) in ethanol (123 mL) at room temperature was added N-Boc-2-hydrazidoethylamine (13 g, 74 mmol). The reaction solution immediately became green in color and was stirred under argon for 1.5 hours then heated to reflux for an additional hour before it was cooled to room temperature and diluted with water. It was then extracted with ethyl acetate. The extractions were washed with water and brine, dried over anhydrous sodium sulfate, filtered and concentrated in vacuo.

The resulting residue was purified with silica gel flash column chromatography (hexanes:ethyl acetate=1:1) and co-eluted material was triturated with hexanes to provide the product as a white solid (4.2 g, 38%): ES-MS m/z 597 ((M+H)⁺); ¹H NMR (d₆-DMSO) δ 0.94 (t, J=7.7 Hz, 6H), 1.04 (t, J=7.6, 6H), 1.34 (s, 18H), 2.28-2.35 (q, 4H), 2.39-2.46 (m, 4H), 3.17-3.23 (m, 4H), 3.95 (t, J3=6.7, 4H), 6.87 (m, 2H).

### Step 3. Preparation of tert-butyl 2-[3,5-diethyl-4-(propylsulfanyl)-1H-pyrazol-1-yl]ethylcarbamate

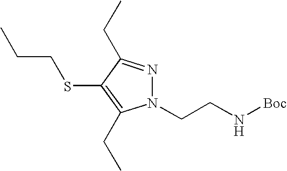

To a solution of the product of step 2 (500 mg, 0.84 mmol) in THF (5.25 mL) at rt was added 0.84 mL of lithium borohydride (2M in THF). The reaction solution was heated to 60° C. for 2.5 hours. Then propyl bromide (0.38 mL, 4.2 mmol) was added and heating was continued at ₆₀° C. for 16 hours before the reaction was cooled to room temperature and quenched over 30 minutes with methanol and 4 drops of hydrochloric acid. It was then diluted with water and saturated aqueous sodium bicarbonate and extracted twice with ethyl acetate. The organic extracts were washed with water and brine, dried over anhydrous sodium sulfate, filtered through a plug of silica gel with 33% ethyl acetate in hexanes and concentrated in vacuo to provide product as a white solid (325 mg, 57%): R_{f}=0.34 (hexanes/ethyl acetate=2/1); ES-MS m/z 342 ((M+H)⁺); ¹H NMR (d₆-DMSO) δ 0.90 (t, J=7.3, 3H), 1.06-1.16 (m, 6H), 1.34 (s, 9H), 1.37-1.44 (m, 21), 2.43 (t, J=6.9, 7.4, 2H), 2.50-2.58 (q, 2H), 2.62-2.69 (q, 2H), 3.18-3.25 (q, 2H), 3.97 (t, J=6.6, 2H), 6.91 (m, 11H).

### Example 109

### Preparation of 2-[3,5-diethyl-4-(propylsulfanyl)-1H-pyrazol-1-yl]ethanamine Trifluoroacetate

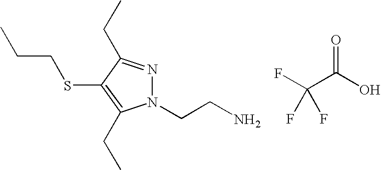

To a solution of Example 108 (284 mg, 0.83 mmol) in dichloromethane (5 mL) at room temperature was added trifluoroacetic acid (1 mL). The reaction solution was stirred for 16 hours then concentrated in vacuo to provide product as a white solid (260 mg, 88%): ES-MS m/z 242 ((M+H)⁺); ¹H NMR (d₆-DMSO) δ 0.91 (t, 3H), 1.09 (t, 3H), 1.16 (t, 3H), 1.38-1.45 (q, 2H), 2.43-2.48 (t, 2H), 2.53-2.61 (q, 2H), 2.66-2.74 (q, 2H), 3.17-3.23 (m, 2H), 4.19 (t, 2H), 7.94 (br. s, 2H).

### Example 110

### Preparation of tert-butyl 2-[3,5-dimethyl-4-(4-piperidinylsulfanyl)-1H-pyrazol-1-yl]ethylcarbamate

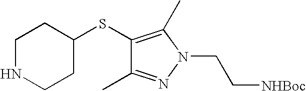

### Step 1. Preparation of 9H-fluoren-9-ylmethyl 4-hydroxy-1-piperidinecarboxylate

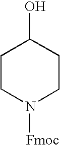

A solution of 4-hydroxypiperidine (5.0 g, 49 mmol) and triethylamine (7.5 mL, 54 mmol) in THF (100 mL) was stirred at 0° C. To this solution was added Fmoc-Cl (13.9 g, 54 mmol) and the resulting mixture was stirred and allowed to come to rt for 18 h. The reaction was concentrated and the resulting slurry redissolved in EtOAc. The resulting solution was washed with 2.0 N HCl in water, saturated aqueous NaCl, dried (MgSO4), concentrated to an oil, and purified on silica using a 1:1 to 1:0 EtOAc:hexane (v/v) gradient as the eluant to yield a white solid (11.05 g, 69%). R_{f}=0.17 (50:50 EtOAc:hex (v/v)); ESLC-MS m/z=324 (MH⁺); ¹H NMR (DMSO-d₆) δ 7.92-7.84 (m, 2H), 7.62-7.56 (m, 2H), 7.45-7.26 (m, 4H), 4.70 (s, 1H), 4.37-4.30 (m, 2H), 4.29-4.21 (m, 1H), 3.67-3.51 (m, 3H), 3.04-2.01 (m, 2H), 1.69-1.54 (m, 2H), 1.27-1.09 (m, 21).

### Step 2. Preparation of 9H-fluoren-9-ylmethyl 4-(acetylsulfanyl)-1-piperidinecarboxylate

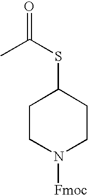

A solution of triphenylphosphine (7.86 g, 30.0 mmol) in THF (100 mL) was stirred and cooled to −78° C. under and argon atmosphere. DEAD (4.72 mL, 30 mmol) was added dropwise, followed by thioacetic acid (2.14 mL, 30 mmol). The resulting mixture was stirred for 10 min at −78° C. Fmoc-4-piperidinethiol (9.0 g, 29 mmol) was then added as a solution in THF (30 mL). The reaction was allowed to warm to rt with stirring over 18 h, then concentrated to a slurry. The slurry was taken up in Et2O, filtered, and the filtrate adsorbed on silica and the product isolated by chromatography on silica using a 6:1 to 3:1 hexane:EtOAC (v/v) gradient as eluant to yield a white solid (5.81 g, 50%). R_{f}=0.40 (20:80 EtOAc:hex (v/v)); ESLC-MS m/z=382 (MH⁺); ¹H NMR (DMSO-d₆) δ 7.89-7.83 (m, 2H), 7.62-7.56 (m, 2H), 7.42-7.35 (m, 2H), 7.35-7.27 (m, 2H), 4.45-4.30 (m, 21), 4.27-4.20 (m, 1H), 3.75-3.42 (m, 3H), 3.06-2.92 (m, 2H), 2.30 (s, 3H), 1.80-1.64 (m, 2H), 1.37-1.17 (m, 2H).

### Step 3. Preparation of 9H-fluoren-9-ylmethyl 4-sulfanyl-1-piperidinecarboxylate

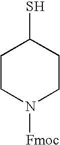

To a suspension of the product of step 2 (2.73 g, 7.16 mmol) in EtOH (25 mL) was added hydrazine monohydrate (0.60 mL, 12 mmol). The reaction gradually became a clear solution. Once clear, the reaction was poured into 1.0 N HCl in water (100 mL) and the resulting mixture was extracted with EtOAc. The organic layers were dried (MgSO4) and concentrated to yield a clear oil (2.45 g, 100%) which was used without further purification. R_{f}=0.40 (20:80 EtOAc:hex (v/v)).

### Step 4. Preparation of 9H-fluoren-9-ylmethyl 4-[(1-acetyl-2-oxopropyl)sulfanyl]-1-Piperidinecarboxylate

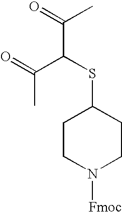

To a solution of fmoc-4-piperidinethiol (173 mg, 0.51 mmol) was stirred in Et₂O (2 mL) was added 3-chloro-2,4-pentanedione. The resulting solution was stirred for 3 h at rt, diluted with Et₂O and then washed once with dilute aqueous HCl. The organic layer was dried (MgSO₄) and concentrated to a clear oil (240 mg) which was used without further purification. R_{f}=0.28 (20:80 EtOAc:hex (v/v)); ESLC-MS m/z=438 (MH⁺).

### Step 5. Preparation of 9H-fluoren-9-ylmethyl 4-[(1-{2-[(tert-butoxycarbonyl)amino]ethyl}-3,5-dimethyl-H-pyrazol-4-yl)sulfanyl]-1-piperidinecarboxylate

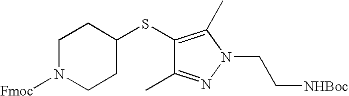

To a solution of the diketone (4.35 g, 9.95 mmol) from step 4 in EtOH (20 mL) was added a solution of N-Boc-3-hydrazidoethylamine (3.5 g, 19.9 mmol) in EtOH (20 mL). The resulting mixture was heated to reflux for 30 min, cooled to rt, and then concentrated. The resulting oil was partitioned between EtOAc and water. The organic layer was collected and the aqueous layer was extracted with EtOAc. The combined organic layers were dried (MgSO4), concentrated, and the residue purified on silica using a gradient from 1:1 to 3:1 EtOAc:hexanes (v/v) to yield a white solid (1.42 g, 25% for two steps). R_{f}=0.59 (EtOAc); ESLC-MS m/z=577 (MH⁺); ¹H NMR (DMSO-d₆) δ 7.89-7.83 (m, 2H), 7.61-7.56 (m, 2H), 7.43-7.36 (m, 2H), 7.34-7.26 (m, 2H), 6.92-6.86 (m, 1H), 4.40-4.28 (m, 2H), 4.26-4.20 (m, 1H), 4.03-3.95 (m, 2H), 3.88-3.62 (m, 2H), 3.25-3.15 (m, 2H), 2.84-2.61 (m, 3H), 2.22 (s, 3H), 2.13 (s, 3H), 1.75-1.62 (m, 2H), 1.33 (s, 9H), 1.39-1.31 (m, 2H).

### Step 6. Preparation of tert-butyl 2-[3,5-dimethyl-4-(4-piperidinylsulfanyl)-1H-pyrazol-1-yl]ethylcarbamate

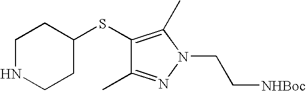

The product of step 5 (1.36 g, 2.36 mmol), 1 M sodium hydroxide (8 mL), and methanol (8 mL) were mixed at rt and left stirring overnight. Water was then added and the mixture was extracted with ethyl acetate (2×25 mL). The combined organic layer was washed with water, brine, and dried (Na₂SO₄). After concentration, 400 mg of crude product (48%) was obtained and used for the next step without purification. ESLC-MS m/z=355 (MH⁺).

### Example 111

### Preparation of tert-butyl 2-{4-[(4-fluorophenyl)sulfanyl]-3,5-dimethyl-1H-pyrazol-1-yl}ethylcarbamate

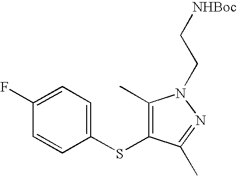

### Step 1. Preparation of 3-[(4-fluorophenyl)sulfanyl]-2,4-pentanedione

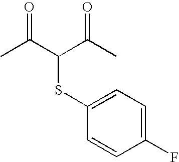

The compound was prepare using the same procedure as Example 1, steps 1 and 2. Product (4.94 g, 98%): GC/MS 227 (M+H)⁺, ¹H NMR (300 MHz, CDCl₃) δ 7.08-6.96 (m, 4H), 2.34 (s, 6H).

### Step 2. Preparation of 4-[(4-fluorophenyl)sulfanyl]-3,5-dimethyl-1H-pyrazole

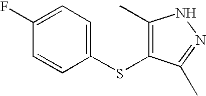

The compound was prepared using the same procedure as Example 1, step 3. Product (4.25 g, 88%). GC/MS 222 (M)⁺, ¹H NMR (300 MHz, CDCl₃) δ 7.00-6.89 (m, 4H), 2.31 (s, 6H).

### Step 3. Preparation of tert-butyl 2-{4-[(4-fluorophenyl)sulfanyl]-3,5-dimethyl-1H-pyrazol-1-yl}ethylcarbamate

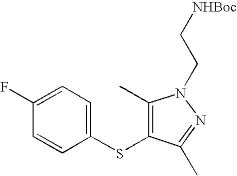

The same as in the procedure for Example 6. Product (0.255 g): R=0.46 (50% EtOAc in Hexanes), ¹H NMR (300 MHz, CDCl₃) δ 6.97-6.87 (m, 4H), 4.88 (m, 1H), 4.15 (t, 2H), 3.54 (q, 2H), 2.25 (s, 3H), 2.19 (s, 3H), 1.42 (s, 9H).

### Example 112

### Preparation of 3,5-dimethyl-4-[(4-nitrophenyl)sulfonyl]-1H-pyrazole

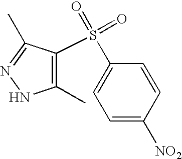

3,5-Dimethyl-4-[(4-nitrophenyl)sulfanyl]-1H-pyrazole (prepared as in Example 1, step 4) was added to a mixture of hydrogen peroxide (10 mL, 0.0865 mol) and acetic acid (57 mL). The mixture was heated to 100° C. and the heat turned off, while the mixture cooled to room temperature, the mixture was concentrated to give 6.54 g of yellow solid (used in the next step without further purification). MS (Electronspray) 282 (M+H)⁺, ¹H NMR (300 MHz, CDCl₃) δ 8.36 (d, 2H), 8.06 (d, 2H), 2.51 (s, 6H).

### Example 113

### Preparation of tert-butyl 2-}4-[(4-fluorophenyl)sulfonyl]-3,5-dimethyl-1H-pyrazol-1-yl}ethylcarbamate

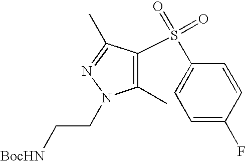

The compound was prepared from Example 111 using the same procedure as Example 112. Product (0.265 g, 61%): MS (Electronspray) 398 (M+H)⁺, ¹H NMR (300 MHz, CDCl₃) δ 7.91-7.86 (m, 2H), 7.26-7.14 (m, 2H), 4.80 (m, 1H), 4.10 (t, 2H), 3.49 (q, 2H), 2.51 (s, 3H), 2.36 (s, 3H), 1.41 (s, 9H).

### Example 114

### Preparation of (2Z)-2-butenedioic Acid Compound with 2-{4-[(4-fluorophenyl)sulfonyl]-3,5-dimethyl-1H-pyrazol-1-yl}ethanamine (1:1)

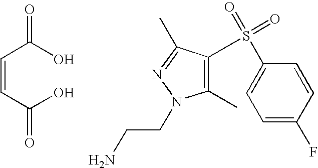

The compound was prepared from Example 113 using the same procedure as Example 32. Product (0.35 g, 96%): ¹H NMR (300 MHz, CD₃OD) δ 7.98-7.93 (m, 2H), 7.34-7.28 (m, 2H), 4.30 (t, 2H), 3.40 (t, 2H), 2.56 (s, 3H), 2.35 (s, 3H).

### Example 115

### Preparation of tert-butyl 2-{3,5-dimethyl-4-[(4-nitrophenyl)sulfonyl]-1H-pyrazol-1-yl}ethylcarbamate

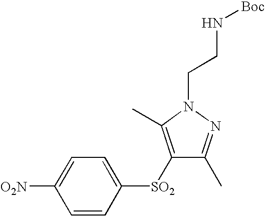

Cesium carbonate (13.9 g, 42.7 mmol) was added to a solution of Example 112 (4 g, 14.2 mmol) and 2-(bromoethyl)carbamic acid, tert-butyl ester (5.74 g, 25.6 mmol) in N,N-dimethylformamide (37 mL). The mixture was stirred at room temperature for 16 h and diluted with ethyl acetate (50 mL) and washed with water (15 mL) and dried over MgSO₄ and concentrated. The product (1.49 g, 86%) was isolated by column chromatography (50% EtOAc in Hexane). R_{f}=0.38 (50% EtOAc in Hexane), MS (Electronspray) 425 (M+H)⁺, ¹H NMR (300 MHz, CDCl₃) δ 8.34 (d, 2H), 8.05 (d, 2H), 4.75 (t, 1H), 4.10 (t, 2H), 3.49 (q, 2H), 2.53 (s, 3H), 2.38 (s, 3H), 1.39 (s, 9H).

### Example 116

### Preparation of tert-butyl 2-{4-[(4-aminophenyl)sulfonyl]-3,5-dimethyl-1H-pyrazol-1-yl}ethylcarbamate

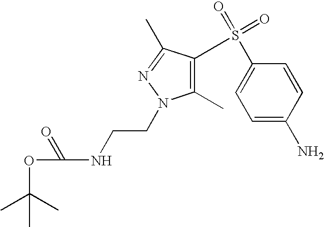

A solution of Example 115 (3.75 g, 8.55 mmol) in ethyl acetate (50 mL) was subjected to hydrogenation using 10% palladium on carbon (0.38 g) at 50 psi of hydrogen for 24 h to give 3.36 g, 100% of product. R_{f}=0.12 (50% EtOAc in Hexane), MS (Electronspray) 395 (M+H)⁺, ¹H NMR (300 MHz, CDCl₃) δ 7.65 (d, 2H), 6.66 (d, 2H), 4.78 (m, 1H), 4.11 (t, 2H), 3.49 (q, 2H), 2.49 (s, 3H), 2.36 (s, 3H), 1.42 (s, 9H).

### Example 117

### Preparation of tert-butyl 2-[4-({4-[(2,2-dimethylpropanoyl)amino]phenyl}sulfonyl)-3,5-dimethyl-1H-pyrazol-1-yl]ethylcarbamate

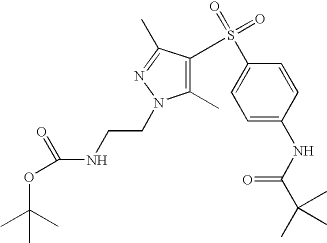

t-Butylcarbonyl chloride (0.091 mL, 0.735 mmol) was added to mixture of Example 116 (0.276 g, 0.700 mmol) and pyridine (0.113 mL, 1.40 mmol) in dichloromethane (3 mL) at room temperature. The mixture was stirred for 5 h, diluted with dichloromethane (15 mL), washed with water (5 mL), dried over MgSO₄ and concentrated. The product was isolated by column chromatography (66% EtOAc in Hexane). MS (Electronspray) 479 (M+H)⁺, ¹H NMR (300 MHz, CDCl₃) δ 7.80 (d, 2H), 7.66 (d, 2H), 4.79 (m, 1H), 4.08 (t, 2H), 3.47 (q, 2H), 2.48 (s, 3H), 2.35 (s, 3H), 1.41 (s, 9H), 1.30 (s, 9H).

### Example 118

### Preparation of N-(4-{[1-(2-aminoethyl)-3,5-dimethyl-1H-pyrazol-4-yl]sulfonyl}phenyl)-2,2-dimethylpropanamide, Trifluoroacetic Acid Salt

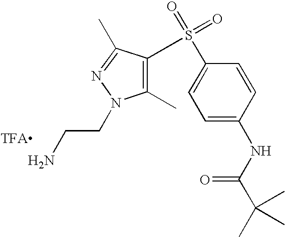

To a solution of Example 117 (0.242 g, 0.506 mmol) in dichloromethane (2 mL) was added trifluoroacetic acid (0.39 mL). The mixture was stirred for 4 h and concentrated under reduced pressure. The residue was triturated with ether to give a cream colored solid (0.284 g, 94%). ¹H NMR (300 MHz, CD₃OD) δ 7.81 (s, 4H), 4.31 (t, 2H), 3.40 (t, 2H), 2.55 (s, 3H), 2.35 (s, 3H), 1.29 (s, 9H).

### Example 119

### Preparation of tert-butyl 2-(4-{[4-(3,3-dimethyl-2,5-dioxo-1-pyrrolidinyl)phenyl]sulfonyl}-3,5-dimethyl-1H-pyrazol-1-yl)ethylcarbamate

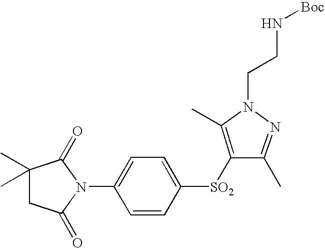

The compound was prepared using the same procedure as Example 113. Product (0.37 g, 72%): R_{f}=0.46 (50% EtOAc in Hexanes), MS (Electronspray) 505 (M+H)⁺, ¹H NMR (300 MHz, CDCl₃) δ 7.96 (d, 2H), 7.51 (d, 2H), 4.81 (m, 1H), 4.12 (t, 2H), 3.49 (q, 2H), 2.75 (s, 2H), 2.51 (s, 3H), 2.39 (s, 3H), 1.44 (s, 9H), 1.42 (s, 6H).

### Example 120

### Preparation of 1-(4-{[1-(2-aminoethyl)-3,5-dimethyl-yl]-pyrazol-4-yl]sulfonyl}phenyl)-3,3-dimethyl-2,5-pyrrolidinedione, Trifluoroacetic Acid Salt

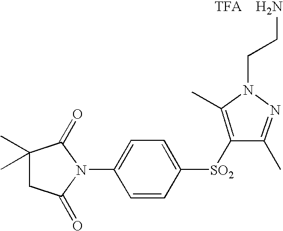

The compound was prepared using the same procedure as Example 118. Product (0.35 g, 96%): MS (Electronspray) 405 (M+H)⁺, RT=2.65, ¹H NMR (300 MHz, CD₃OD) δ 8.00 (d, 2H), 7.56 (d, 2H), 4.30 (t, 2H), 3.40 (t, 2H), 2.77 (s, 2H), 2.57 (s, 3H), 2.38 (s, 3H), 1.39 (s, 6H).

### Example 121

### Preparation of tert-butyl 2-{3,5-dimethyl-4-[(4-nitrophenyl)sulfonyl]-1H-pyrazol-1-yl}ethyl(methyl)carbamate

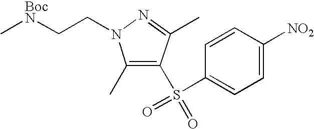

The compound was prepared using the same procedure as Example 22. Product (0.47 g, 33%): R_{f}=0.51 (50% EtOAc in Hexane), MS (Electronspray) 439 (M+H)⁺, ¹H NMR (300 MHz, CDCl₃) δ 8.33 (d, 2H), 8.04 (d, 2H), 4.19-4.08 (m, 2H), 3.54 (t, 2H), 2.75-2.37 (m, 11H), 1.39 (s, 15H).

### Example 122

### Preparation of tert-butyl 2-{4-[(4-aminophenyl)sulfonyl-3,5-dimethyl-1H-pyrazol-1-yl}ethyl(methyl)carbamate

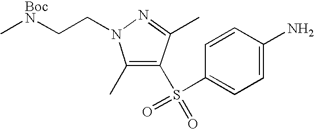

The compound was prepared using the same procedure as Example 116. Product (0.43 g, 100%): R_{f}=0.11 (50% EtOAc in Hexane), MS (Electronspray) 408 (M+H)⁺, ¹H NMR (300 MHz, CDCl₃) δ 7.63 (d, 2H), 6.64 (d, 2H), 4.11-4.06 (m, 2H), 3.52 (t, 2H), 2.63-2.36 (m, 9H), 1.41 (s, 9H).

### Example 123

### Preparation of tert-butyl 2-[4-({4-[(2,2-dimethylpropanoyl)amino]phenyl}sulfonyl)-3,5-dimethyl-1H-pyrazol-1-yl]ethyl(methyl)carbamate

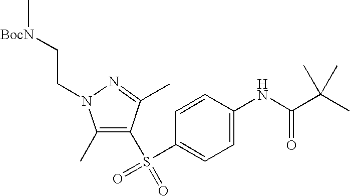

The compound was prepared using the same procedure described for Example 117. Product (0.43 g, 100%): R=0.50 (50% EtOAc in Hexane), MS (Electronspray) 493 (M+H)⁺, ¹H NMR (300 MHz, CDCl₃) δ 7.80 (d, 2H), 7.66 (d, 2H), 4.15-4.04 (m, 2H), 3.52 (t, 2H), 2.63-2.36 (m, 9H), 1.42 (s, 9H), 1.33 (s, 9H).

### Example 124

### Preparation of N-[4-({3,5-dimethyl-1-[2-(methylamino)ethyl]-1H-pyrazol-4-yl}sulfonyl)phenyl]-2,2-dimethylpropanamide

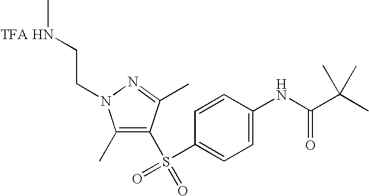

The compound was prepared using the same procedure described for Example 118. Product (0.204 g, 99%): ¹H NMR (300 MHz, CD₃OD) δ 7.82 (s, 4H), 4.35 (t, 2H), 3.50-3.45 (m, 2H), 2.74 (s, 3H), 2.56 (s, 3H), 2.36 (s, 3H), 1.29 (s, 9H).

### Example 125

### Preparation of tert-butyl 3,5-dimethyl-4-[(4-nitrophenyl)sulfonyl]-1H-pyrazole-1-carboxylate

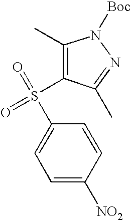

Di-tert-butyl dicarbonate (0.73 g, 3.24 mmol) was added in one portion to a solution of Example 112 (0.8 g, 3.21 mmol) and dimethylamino pyridine (few crystals) in acetonitrile (6.5 mL). The mixture was stirred at room temperature for 8 h and concentrated. The product (0.89 g, 73%) was isolated by column chromatography (50% EtOAc in Hexanes). R_{f}=0.81 (50% EtOAc in Hexane). ¹H NMR (300 MHz, CDCl₃) δ 8.38 (d, 2H), 8.07 (d, 2H), 2.85 (s, 3H), 2.46 (s, 3H), 1.64 (s, 6H).

### Example 126

### Preparation of tert-butyl 4-[(4-aminophenyl)sulfonyl]-3,5-dimethyl-1H-pyrazole-1-carboxylate

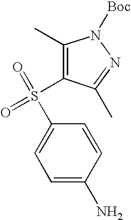

The compound was prepared using the same procedure described for Example 116. Product (0.76 g, 92%): R_{f}=0.81 (50% EtOAc in Hexane), ¹H NMR (300 MHz, CDCl₃) δ 7.65 (d, 2H), 6.67 (d, 2H), 2.80 (s, 3H), 2.42 (s, 3H), 1.63 (s, 6H).

### Example 127

### Preparation of 1-{4-[(3,5-dimethyl-1H-pyrazol-4-yl)sulfonyl]phenyl}-3,3-dimethyl-2,5-pyrrolidinedione

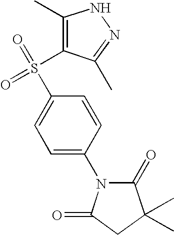

To a solution of the aniline Example 126 (0.736 g, 2.09 mmol) and triethylamine (0.12 mL, 0.838 mmol) in pyridine (10 mL) and toluene (10 mL) was added 2,2-dimethylsuccinic anhydride (0.402 g, 3.14 mmol). The mixture was refluxed under argon overnight and concentrated under reduced pressure. The product (0.56 g, 74%) was isolated by column chromatography (50% EtOAc in Hexane). R_{f}=0.20, MS (Electronspray) 390 (M+H)⁺, ¹H NMR (300 MHz, CDCl₃) δ 7.96 (d, 2H), 7.51 (d, 2H), 2.72 (s, 21), 2.47 (s, 6H), 1.44 (s, 6H).

### Example 128

### Preparation of tert-butyl 2-(4-{[4-(3,3-dimethyl-2,5-dioxo-1-pyrrolidinyl)phenyl]sulfonyl}-3,5-dimethyl-1H-pyrazol-1-yl)ethyl(methyl)carbamate

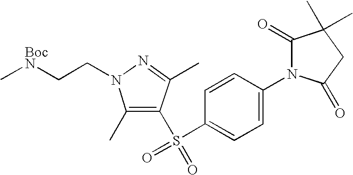

Methanesulfonyl chloride (0.27 mL, 3.46 mmol) was added to a cooled (0° C.) solution of (2-hydroxy-ethyl)-methyl-carbamic acid t-butyl ester (0.61 g, 3.46 mmol) and triethylamine (0.48 mL, 3.46 mmol) in dichloromethane (3.4 mL). The resulting cloudy mixture was stirred at 0° C. for 30 min and concentrated. The residue was taken up in ethyl acetate (20 mL) and filtered through a plug of silica gel. The filtrate was concentrated and dissolved in N,N-dimethylformamide (3 mL), and the solution added to a mixture of Example 17 (0.25 g, 0.692 mmol) and cesium carbonate (1.8 g, 5.53 mmol) in N,N-dimethylformamide (3.4 mL). The resulting yellow suspension was heated at 58° C. for 16 h, cooled and diluted with ethyl acetate (50 mL) and water (10 mL). The organic washed with water (2×10 mL), dried over MgSO₄ and concentrated. The product (0.222 g, 62%) was isolated by column chromatography (50% EtOAc in Hexane). R_{f}=0.18 (50% EtOAc in Hexane), MS (Electronspray) 519 (M+H)⁺, ¹H NMR (300 MHz, CDCl₃) δ 8.33 (d, 2H), 8.04 (d, 2H), 4.19-4.08 (m, 2H), 3.54 (t, 2H), 2.75-2.37 (m, 11H), 1.39 (s, 15H).

### Example 129

### Preparation of (2Z)-2-butenedioic Acid Compound with 1-[4-({3,5-dimethyl-1-[2-(methylamino)ethyl]-1H-pyrazol-4-yl}sulfonyl)phenyl]-3,3-dimethyl-2,5-pyrrolidinedione (1:1)

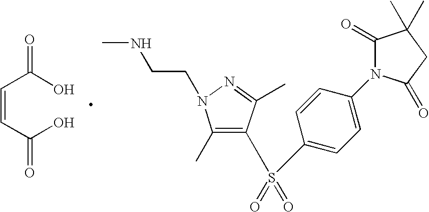

The compound was prepared using the same procedure described for Example 24. Product (0.047 g, 21%): ¹H NMR (300 MHz, CD₃OD) δ 7.96 (d, 2H), 7.53 (d, 2H), 6.33 (s, 2H), 4.34 (t, 2H), 3.56 (s, 2H), 2.83 (s, 3H), 2.75 (s, 2H), 2.53 (s, 3H), 2.36 (s, 3H), 1.41 (s, 6H).

### Example 130

### Preparation of N-(2-{3,5-dimethyl-4-[(4-nitrophenyl)sulfanyl]-1H-pyrazol-1-yl}ethyl)-N,N-dimethylamine

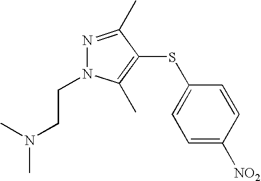

To a suspension of Example 112 (1 g, 4.01 mmol) in acetonitrile (20 mL) was added sodium hydroxide (0.642 g, 16.04 mmol). The mixture was stirred under argon for 30 min at room temperature. 2-Dimethylaminoethyl chloride hydrochloride (0.722 g, 5.01 mol) was added, followed by tetrabutylammonium hydrogen sulfate (0.054 g, 0.160 mmol), the reaction mixture was stirred at reflux for 1.5 h and diluted with ethyl acetate (100 mL), dried over Na₂SO₄, filtered through a bed of Celite®. The filtrate was concentrated. The residue was dissolved in ethyl acetate (20 mL) and passed through a silica gel plug, using 10% methanol in ethyl acetate as the eluant. The eluants were concentrated to give 1.096 g, 85% of product. R_{f}=0.15 (EtOAc), MS (Electronspray) 321 (M+H)⁺, ¹H NMR (300 MHz, CDCl₃) δ 8.05 (d, 2H), 7.03 (d, 2H), 4.17 (t, 31H), 2.76 (t, 3H), 2.31 (s, 6H), 2.28 (s, 3H), 2.19 (s, 3H).

### Example 131

### Preparation of N-(2-{3,5-dimethyl-4-[(4-nitrophenyl)sulfonyl]-1H-pyrazol-1-yl}ethyl)-N,N-dimethylamine

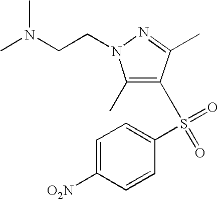

Hydrogen peroxide (0.74 mL, 7.135 mmol) was added slowly to a cooled (0° C.) solution of trifluoroacetic anhydride (3.34 mL, 23.6 mmol) in dichloromethane (13 mL), and the mixture stirred for 20 min. A solution of Example 130 in dichloromethane (6.6 mL) was added dropwise and the stirring continued at 0° C. for 1 h and at room temperature for 30 min. The mixture was diluted with ether (65 mL) and washed sodium hydroxide (2N, 65 mL). The aqueous layer was extracted with ether and the combined organic layer were washed with Na₂SO₃ (20 mL), water (20 mL) and saturated NaCl (20 mL) and dried over Na₂SO₄ and concentrated to give a greenish yellow solid (0.372 g, 46%, used in the next step without further purification). MS (Electronspray) 353 (M+H)⁺, ¹H NMR (300 MHz, CDCl₃) δ 8.33 (d, 2H), 8.03 (d, 2H), 4.05 (t, 3H), 2.66 (t, 3H), 2.54 (s, 3H), 2.37 (s, 3H), 2.24 (s, 6H).

### Example 132

### Preparation of N-(2-{4-[(4-aminophenyl)sulfonyl]-3,5-dimethyl-1H-pyrazol-1-yl}ethyl)-N,N-dimethylamine

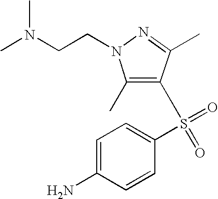

The compound was prepared using the same procedure described for Example 116. Product (0.161 g, 47%): MS (Electronspray) 323 (M+H)⁺, ¹H NMR (300 MHz, CDCl₃) δ 7.58 (d, 2H), 6.60 (d, 2H), 4.04 (t, 3H), 2.66 (t, 3H), 2.48 (s, 3H), 2.33 (s, 3H), 2.25 (s, 6H).

### Example 133

### Preparation of N-[4-({1-[2-(dimethylamino)ethyl]-3,5-dimethyl-1H-pyrazol-4-yl}sulfonyl)phenyl]methanesulfonamide

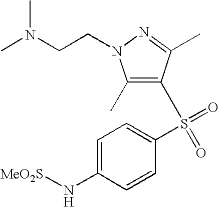

The compound was prepared using the same procedure described for Example 117. Product (0.04 g, 28%): MS (Electronspray) 407 (M+H)⁺, ¹H NMR (300 MHz, CDCl₃) δ 7.80 (d, 2H), 7.66 (d, 2H), 4.20 (t, 3H), 2.89 (t, 3H), 2.54 (s, 3H), 2.39 (s, 6H), 2.35 (s, 3H).

### Example 134

### Preparation of I-butyl (1S)-2-{3,5-dimethyl-4-[(4-nitrophenyl)sulfonyl]-1I-pyrazol-1-yl}-1-methylethylcarbamate

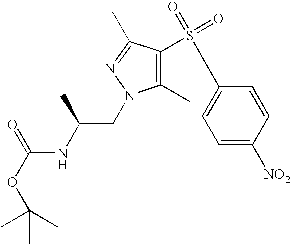

The compound was prepared using the same procedure described for Example 115. Product (0.49 g, 32%): R=0.32 (50% EtOAc in Hexane), MS (Electronspray) 439 (M+H)⁺, ¹H NMR (300 MHz, CDCl₃) δ 8.33 (d, 2H), 8.05 (d, 2H), 4.65 (m, 1H), 4.13-3.95 (m, 3H), 2.58 (s, 3H), 2.37 (s, 3H), 1.33 (s, 9H), 1.18 (d, 3H).

### Example 135

### Preparation of tert-butyl (1S)-2-{4-[(4-aminophenyl)sulfonyl]-3,5-dimethyl-1H-pyrazol-1-yl}-1-methylethylcarbamate

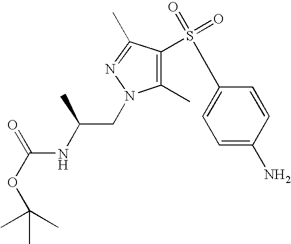

The compound was prepared using the same procedure described for Example 116. Product (0.345 g, 100%): R_{f}=0.13 (50% EtOAc in Hexane), MS (Electronspray) 408 (M)⁺, ¹H NMR (300 MHz, CDCl₃) δ 7.65 (d, 2H), 6.65 (d, 2H), 4.65 (m, 1H), 4.13-3.95 (m, 3H), 2.58 (s, 3H), 2.37 (s, 3H), 1.33 (s, 9H), 1.18 (d, 3H).

### Example 136

### Preparation of tert-butyl (1S)-2-[4-({4-[(2,2-dimethylpropanoyl)amino]phenyl}sulfonyl)-3,5-dimethyl-1H-pyrazol-1-yl]-1-methylethylcarbamate

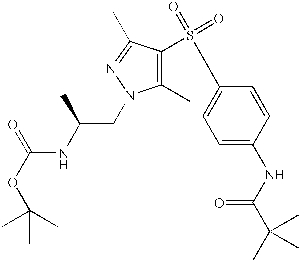

The compound was prepared using the same procedure described for Example 117. Product (0.105 g, 58%): MS (Electronspray) 493 (M+H)⁺, ¹H NMR (300 MHz, CDCl₃) δ 7.81 (d, 2H), 7.65 (d, 2H), 4.77 (d, 1H), 4.13-3.92 (m, 3H), 2.53 (s, 3H), 2.35 (s, 3H), 1.38 (s, 9H), 1.32 (s, 9H), 1.13 (d, 3H).

### Example 137

### Preparation of N-[4-({1-[(2S)-2-aminopropyl]-3,5-dimethyl-1H-pyrazol-4-yl}sulfonyl)phenyl]-2,2-dimethylpropanamide

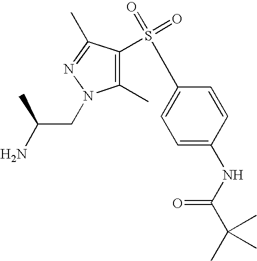

The compound was prepared using the same procedure described for Example 118. Product (0.132 g, 100%): ¹H NMR (300 MHz, CD₃OD) δ 7.76 (d, 2H), 7.68 (d, 2H), 4.24-4.10 (m, 2H), 3.88 (m, 1H), 2.48 (s, 3H), 2.29 (s, 3H), 1.34 (d, 3H), 1.29 (s, 9H).

### Example 138

### Preparation of tert-butyl (1S)-2-(4-{[4-(1,3-dioxo-1,3-dihydro-2H-isoindol-2-yl)phenyl]sulfonyl}-3,5-dimethyl-1H-pyrazol-1-yl)-1-methylethylcarbamate

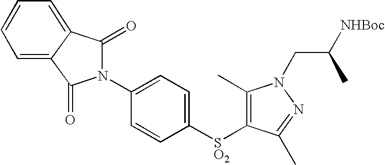

### Step: 1 Preparation of 2-{4-[(3,5-dimethyl-1H-pyrazol-4-yl)sulfonyl]phenyl}-1H-isoindole-1,3(2H)-dione

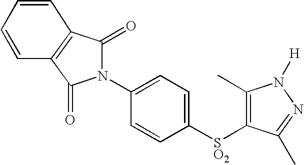

To a round bottom equipped with a condenser under argon was added Example 126 (422 mg, 1.20 mmol) and phthalic anhydride (279 mg, 1.72 mmol) dissolved in toluene. p-Toluenesulfonic acid monohydrate (25 mg, 0.13 mmol) was added to the reaction mixture and was stirred for 18 hours at 115° C.

### Step: 2 Preparation of tert-butyl (1S)-2-(4-{[4-(1,3-dioxo-1,3-dihydro-2H-isoindol-2-yl)phenyl]sulfonyl}-3,5-dimethyl-1H-pyrazol-1-yl)-1-methylethylcarbamate

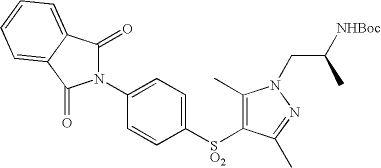

To a round bottom equipped with a condenser under argon was added the compound prepared in step 1 (228 mg, 0.59 mmol) dissolved in methyl sulfoxide in N,N-dimethylformamide (2.5 mL). Sodium Hydride (35 mg, 0.87 mmol) was then added to the solution and let stir for 10 minutes. Mesylate (460 mg, 1.82 mmol) was then added to the reaction mixture, which was then heated to 60° C. for 18 hours. Water was then added and the mixture was extracted with ethyl ether (3×10 mL). The combined organic layers were washed with brine (2×15 mL), dried over anhydrous magnesium sulfate and concentrated under reduced pressure.

### Example 139

### Preparation of 2-[4-({1-[(2S)-2-aminopropyl]-3,5-dimethyl-1H-pyrazol-4-yl}sulfonyl)phenyl]-1H-isoindole-1,3(2H)-dione, Trifluoroacetic Acid Salt

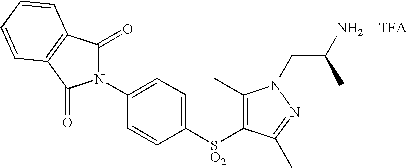

To a solution of Example 138 (82 mg, 0.15 mmol) in dichloromethane was added trifluoroacetic acid (1.5 mL, 19.47 mmol) at room temperature and let stir for 1.5 hours. The reaction mixture was then concentrated under reduced pressure after which ethyl ether was added to precipitate a white solid, which was then filtered and washed with cold ethyl ether.

### Example 140

### Preparation of tert-butyl (1S)-2-{3,5-dimethyl-4-[(4-nitrophenyl)sulfanyl]-1H-pyrazol-1-yl}-1-methylethylcarbamate

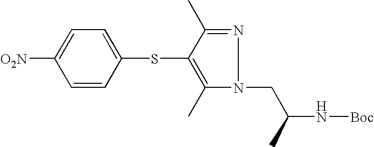

To a solution of 3-[(4-nitrophenyl)sulfanyl]-2,4-pentanedione, prepared as in steps 1 and 2, Example 1 (1.07 g, 4.2 mmol) in ethanol (10 mL) under argon was added tert-butyl (1R)-1-hydrazinoethylcarbamate (1.2 g, 6.3 mmol). This reaction mixture was stirred for 10 min. prior to the addition of acetic acid (5 drops). The reaction mixture was then heated to 95° C. for 1.5 h. This mixture was then concentrated under reduced pressure to yield the desired product.

### Example 141

### Preparation of tert-butyl (1S)-2-{3,5-dimethyl-4-[(4-nitrophenyl)sulfonyl]-1H-pyrazol-1-yl}-1-methylethylcarbamate

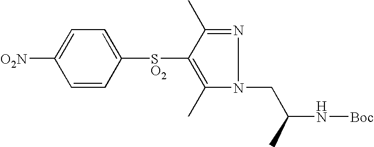

To a solution of Example 140 in dichloromethane was added MCPBA and the mixture was stirred for 18 h under argon. Sodium thiosulfate (35 mL) and saturated sodium bicarbonate (70 mL) along with 50 mL of dichloromethane was added to the mixture and was stirred for 0.5 h. Extracted with dichloromethane (3×50 mL) and washed with water. This was then dried over magnesium sulfate and concentrated under reduced pressure to yield the desired product (1.79 g, 97%).

### Example 142

### Preparation of tert-butyl (1S)-2-{4-[(4-aminophenyl)sulfonyl]-3,5-dimethyl-1H-pyrazol-1-yl}-1-methylethylcarbamate

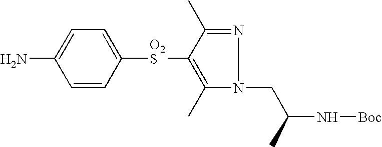

To a solution of Example 141 ethanol was added Raney Nickel and was then equipped with a hydrogen balloon. Let stir for 2 h and was then filtered and washed with ethanol to yield the desired product (3.3 g, 85%).

### Example 143

### Preparation of 2-{3,5-diethyl-4-[(4-fluorophenyl)sulfonyl]-1H-pyrazol-1-yl}ethylamine

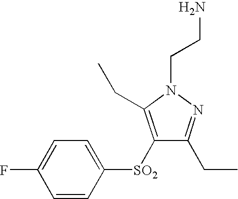

### Step 1: Preparation of 3,5-diethyl-4-[(4-fluorophenyl)sulfonyl]-1H-pyrazole

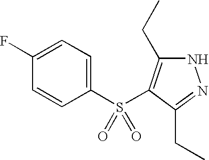

The compound was prepared using the same procedure described for Example 112. Product (3.83 g, 98%): MS (Electronspray) 283 (M+H)⁺, ¹H NMR (300 MHz, CDCl₃) δ 7.89 (d, 2H), 7.19 (d, 2H), 2.90 (q, 4H), 1.24 (t, 6H).

### Step 2: Preparation of 2-{3,5-diethyl-4-[(4-fluorophenyl)sulfonyl]-1H-pyrazol-1-yl}ethylamine

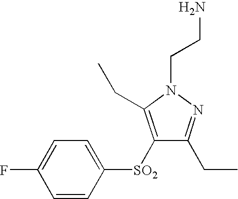

The compound was prepared using the same procedure described for Example 130. Product (0.063 g, 53%): MS (Electronspray) 326 (M+H)⁺, ¹H NMR (300 MHz, CDCl₃) δ 7.89 (d, 2H), 7.19 (d, 2H), 4.03 (t, 2H), 3.17 (t, 2H), 2.98 (q, 4H), 2.78 (q, 4H), 1.23-1.15 (m, 6H).

### Example 144

### Preparation of 2-{3,5-diethyl-4-[(4-fluorophenyl)sulfonyl]-1H-pyrazol-1-yl}ethylamine, Dihydrochloride

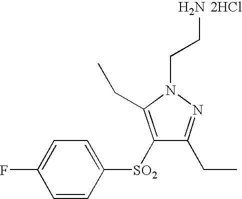

To a solution of Example 143 (0.056 g, 0.172 mmol) in ether (2 mL) was added HCl (0.43 mL, 2M) in ether at room temperature. The mixture was stirred at room temperature for 2 h and concentrated to give 0.065 g, 96% of product. ¹H NMR (300 MHz, DMSO) δ 7.92 (d, 2H), 7.43 (d, 2H), 4.29 (t, 2H), 3.20 (q, 2H), 2.94 (q, 4H), 2.68 (q, 4H), 1.13-1.02 (m, 6H).

### Example 145

### Preparation of tert-butyl 2-{3,5-diethyl-4-[(4-nitrophenyl)sulfonyl]-1H-pyrazol-1-yl}ethylcarbamate

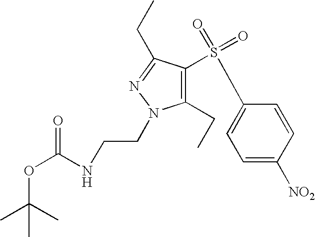

The compound was prepared using the same procedure described for Example 115. Product (1.26 g, 86%): R=0.46 (50% EtOAc in Hexane), MS (Electronspray) 453 (M+H)⁺, ¹H NMR (300 MHz, CDCl₃) δ 8.33 (d, 2H), 8.04 (d, 2H), 4.85 (m, 1H), 4.10 (t, 2H), 3.56 (q, 2H), 2.95 (q, 2H), 2.77 (q, 2H), 1.47 (s, 9H), 1.28-1.16 (m, 6H).

### Example 146

### Preparation of tert-butyl 2-{4-[(4-aminophenyl)sulfonyl]-3,5-diethyl-1H-pyrazol-1-yl}ethylcarbamate

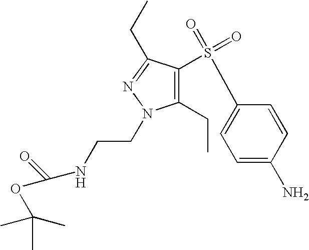

The compound was prepared using the same procedure described for Example 116. Product (0.13 g, used without further purification). R=0.19 (50% EtOAc in Hexane), MS (Electronspray) 422 (M+H)⁺, ¹H NMR (300 MHz, CDCl₃) δ 7.64 (d, 2H), 6.65 (d, 2H), 4.90 (m, 1H), 4.06 (t, 2H), 3.55 (q, 2H), 2.93 (q, 2H), 2.78 (q, 2H), 1.42 (s, 9H), 1.27-1.12 (m, 6H).

### Example 147

### Preparation of tert-butyl 2-[4-({4-[(2,2-dimethylpropanoyl)amino]phenyl}sulfonyl)-3,5-diethyl-1H-pyrazol-1-yl]ethylcarbamate

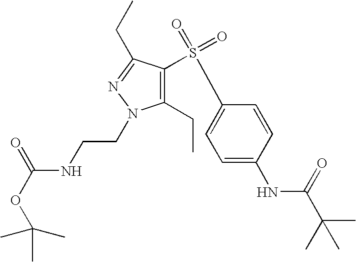

The compound was prepared using the same procedure described for Example 117. Product (0.22 g, 60%): R=0.50 (50% EtOAc in Hexane), MS (Electronspray) 507 (M+H)⁺, ¹H NMR (300 MHz, CDCl₃) δ 7.79 (d, 2H), 7.65 (d, 2H), 4.91 (m, 1H), 4.12-4.03 (m, 2H), 3.54 (q, 2H), 2.92 (q, 2H), 2.76 (q, 2H), 1.41 (s, 9H), 1.30 (s, 9H), 1.25-1.12 (m, 6H).

### Example 148

### Preparation of N-(4-{[1-(2-aminoethyl)-3,5-diethyl-1H-pyrazol-4-yl]sulfonyl}phenyl)-2,2-dimethylpropanamide, Trifluoroacetic Acid Salt

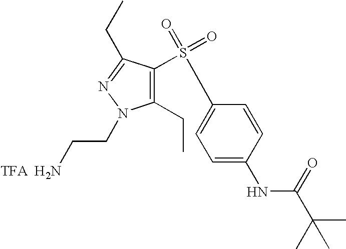

The compound was prepared using the same procedure described for Example 118. Product (0.20 g, 98%): ¹H NMR (300 MHz, CD₃OD) δ 7.80 (s, 4H), 4.33 (t, 2H), 3.43 (t, 2H), 3.01 (q, 2H), 2.80 (q, 2H), 1.29 (s, 9H), 1.23-1.15 (m, 6H).

### Example 149

### Preparation of tert-butyl 2-(4-{[4-(3,3-dimethyl-2,5-dioxo-1-pyrrolidinyl)phenyl]sulfonyl}-3,5-diethyl-1H-pyrazol-1-yl)ethylcarbamate

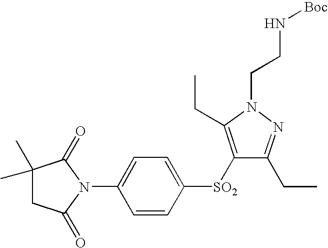

The compound was prepared using the same procedure described for Example 127. Product (0.14 g, 28%): MS (Electronspray) 533 (M+H)⁺.

### Example 150

### Preparation of 1-(4-{[1-(2-aminoethyl)-3,5-diethyl-1H-pyrazol-4-yl]sulfonyl}phenyl)-3,3-dimethyl-2,5-pyrrolidinedione, Trifluoroacetic Acid Salt

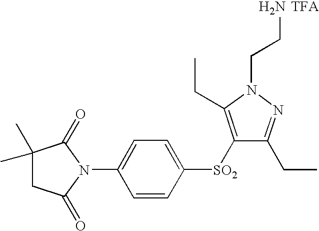

The compound was prepared using the same procedure described for Example 118. Product (0.14 g, 97%): MS (Electronspray) 433 (M+H)⁺, RT=2.77. ¹H NMR (300 MHz, CD₃OD) δ 8.00 (d, 2H), 7.58 (d, 2H), 4.34 (t, 2H), 3.44 (t, 2H), 3.03 (q, 2H), 2.83 (q, 2H), 2.78 (s, 2H), 1.40 (s, 6H), 1.25-1.15 (m, 6H).

### Example 151

### Preparation of tert-butyl 2-(4-{[4-(1,3-dioxo-1,3 dihydro-2H-isoindol-2-yl)phenyl]sulfonyl}-3,5-diethyl-1H-pyrazol-1-yl)ethylcarbamate

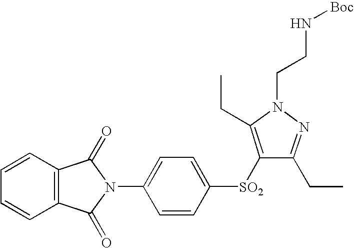

### Step 1: Preparation of tert-butyl 3,5-diethyl-4-[(4-nitrophenyl)sulfonyl]-1H-pyrazole-1-carboxylate

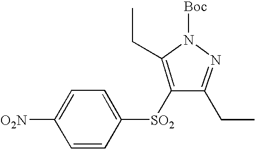

The compound was prepared using the same procedure described for Example 125. Product (3.82 g, 88%): R_{f}=0.72 (50% EtOAc in Hexanes). MS (Electronspray) 410 (M+H)⁺. ¹H NMR (300 MHz, CDCl₃) δ 8.36 (d, 2H), 8.06 (d, 2H), 3.29 (q, 4H), 2.85 (q, 4H), 1.66 (s, 9H), 1.28 (t, 3H), 1.23 (t, 3H).

### Step 2: Preparation of tert-butyl 4-[(4-aminophenyl)sulfonyl]-3,5-diethyl-1H-pyrazole-1-carboxylate

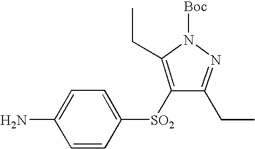

The compound was prepared from the compound of step 1, using the same procedure described for Example 116. Product (2.87 g, 85%): R_{f}=0.28 (50% EtOAc in Hexanes). MS (Electronspray) 380 (M+H)⁺. ¹H NMR (300 MHz, CDCl₃) δ 7.64 (d, 2H), 6.55 (d, 2H), 3.28 (q, 4H), 2.85 (q, 4H), 1.64 (s, 9H), 1.26 (t, 3H), 1.18 (t, 3H).

### Step 3: Preparation of 2-{4-[(3,5-diethyl-1H-pyrazol-4-yl)sulfonyl]phenyl}-1H-isoindole-1,3(2H)-dione

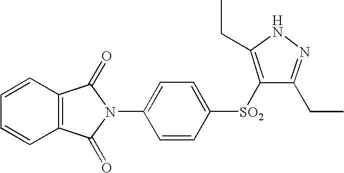

The compound was prepared from the compound of step 2, using the same procedure described for Example 127. Product (0.2 g): MS (Electronspray) 410 (M+H)⁺, ¹H NMR (300 MHz, CDCl₃) δ 8.03-7.97 (m, 4H), 7.84-7.82 (m, 2H), 7.70-7.66 (m, 2H), 2.99-2.92 (m, 4H), 1.33-1.27 (m, 6H).

### Step 4: Preparation of tert-butyl 2-(4-{[4-(1,3-dioxo-1,3-dihydro-2H-isoindol-2-yl)phenyl]sulfonyl}-3,5-diethyl-1H-pyrazol-1-yl)ethylcarbamate

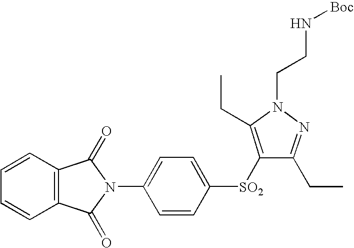

The compound was prepared from the compound of step 3 using the same procedure described for Example 115. Product (0.017 g, 6%): R_{f}=0.27 (50% EtOAc in Hexane), MS (Electronspray) 553 (M+H)⁺, ¹H NMR (300 MHz, CDCl₃) δ 8.02-7.95 (m, 4H), 7.84-7.81 (m, 2H), 7.68-7.65 (m, 2H), 4.93 (m, 1H), 4.09 (m, 2H), 3.58 (m, 2H), 2.96 (q, 2H), 2.80 (q, 2H), 1.42 (s, 91), 1.26-1.18 (m, 6H).

### Example 152

### Preparation of 2-(4-{[1-(2-aminoethyl)-3,5-diethyl-1H-pyrazol-4-yl]sulfonyl}phenyl)-1H-isoindole-1,3(2H)-dione, Trifluoroacetic Acid Salt

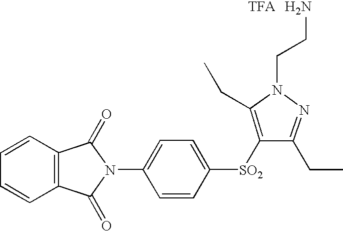

The compound was prepared using the same procedure described for Example 124. Product (0.016 g, 89%): MS (Electronspray) 453 (M+H)⁺, RT=2.88. ¹H NMR (300 MHz, CDCl₃) δ 8.05-7.88 (m, 6H), 7.75 (d, 2H), 4.35 (t, 2H), 3.45 (t, 2H), 3.05 (q, 2H), 2.86 (q, 2H), 1.29-1.20 (m, 6H).

### Example 153

### Preparation of tert-butyl 2-{3,5-diethyl-4-[(4-fluorophenyl)sulfonyl]-1H-pyrazol-1-yl}ethyl(methyl)carbamate

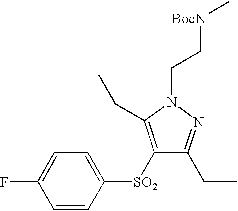

The compound was prepared using the same procedure described for Example 128. Product (0.202 g, 32%): MS (Electronspray) 440 (M+H)⁺, ¹H NMR (300 MHz, CDCl₃) δ 7.90-7.86 (m, 2H), 7.18-7.13 (m, 2H), 4.97 (m, 1H), 4.10-3.98 (m, 3H), 2.97 (q, 2H), 2.75 (q, 2H), 1.37 (s, 9H), 1.23-1.14 (m, 9H).

### Example 154

### Preparation of N-(2-{3,5-diethyl-4-[(4-fluorophenyl)sulfonyl]-1H-pyrazol-1-yl}-ethyl)-N-methylamine, Trifluoroacetic Acid Salt

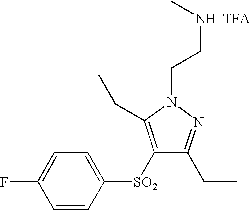

The compound was prepared using the same procedure described for Example 124. Product (0.08 g, 100%): ¹H NMR (300 MHz, CD₃OD) δ 7.97-7.93 (m, 2H), 7.34-7.29 (m, 2H), 4.33-4.19 (m, 2H), 3.87-3.81 (in 1H), 3.10-2.97 (m, 2H), 2.80 (q, 2H), 1.31 (d, 3H), 1.23-1.14 (m, 6H).

### Example 155

### Preparation of tert-butyl 2-{3,5-diethyl-4-[(4-nitrophenyl)sulfonyl]-1H-pyrazol-1-yl}ethyl(methyl)carbamate

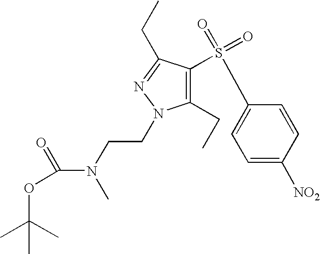

The compound was prepared using the same procedure described for Example 128. Product (0.49 g, 32%): MS (Electronspray) 467 (M+H)⁺, ¹H NMR (300 MHz, CDCl₃) δ 8.33 (d, 2H), 8.03 (d, 2H), 4.15 (t, 2H), 3.62 (t, 2H), 2.94 (q, 2H), 2.78 (q, 2H), 2.63 (d, 3H), 1.42 (s, 9H), 1.24-1.20 (m, 6H).

### Example 156

### Preparation of tert-butyl 2-{4-[(4-aminophenyl)sulfonyl]-3,5-diethyl-1H-pyrazol-1-yl}ethyl(methyl)carbamate

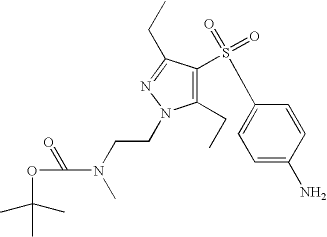

The compound was prepared using the same procedure described for Example 116. Product (0.50 g, used without further purification): R_{f}=0.29 (EtOAc), MS (Electronspray) 437 (M+H)⁺, ¹H NMR (300 MHz, CDCl₃) δ 7.64 (d, 2H), 6.64 (d, 2H), 4.13-4.06 (m, 2H), 3.60 (t, 2H), 2.91 (q, 2H), 2.78 (q, 2H), 2.57 (d, 3H), 1.43 (s, 9H), 1.26-1.14 (m, 6H).

### Example 157

### Preparation of tert-butyl 2-{4-([4-[(2,2-dimethylpropanoyl)amino]phenyl}sulfonyl)-3,5-diethyl-1H-pyrazol-1-yl]ethyl(methyl)carbamate

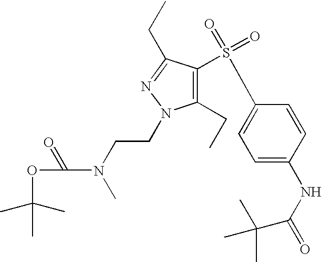

The compound was prepared using the same procedure described for Example 117. Product (0.175 g, 100%): MS (Electronspray) 521 (M+H)⁺, ¹H NMR (300 MHz, CDCl₃) δ 7.80 (d, 2H), 7.65 (d, 2H), 4.14-4.08 (m, 2H), 3.60 (t, 2H), 2.91 (q, 2H), 2.78 (q, 2H), 2.58 (d, 3H), 1.32 (s, 9H), 1.27 (s, 9H), 1.24-1.17 (m, 6H).

### Example 158

### Preparation of N-[4-({3,5-diethyl-1-[2-(methylamino)ethyl]-1H-pyrazol-4-yl}sulfonyl)phenyl]-2,2-dimethylpropanamide

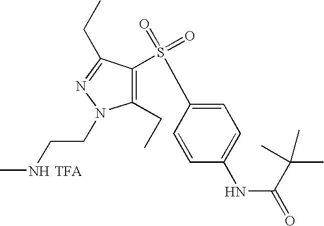

The compound was prepared using the same procedure described for Example 124. Product (0.163 g, 91%): ¹H NMR (300 MHz, CD₃OD) δ 7.81 (s, 4H), 4.38 (t, 2H), 3.50 (t, 2H), 3.02 (q, 2H), 2.80 (q, 2H), 2.76 (s, 3H), 1.29 (s, 9H), 1.23-1.15 (m, 6H).

### Example 159

### Preparation of tert-butyl 2-(4-{[4-(3,3-dimethyl-2,5-dioxo-1-pyrrolidinyl)phenyl]sulfonyl}-3,5-diethyl-1H-pyrazol-1-yl)ethyl(methyl)carbamate

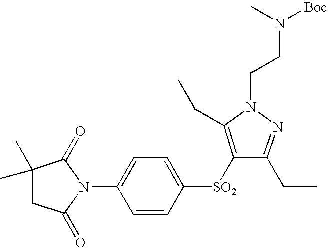

The compound was prepared using the same procedure described for Example 128. Product (0.31 g, 74%): MS (Electronspray) 547 (M+H)⁺, ¹H NMR (300 MHz, CDCl₃) δ 7.96 (d, 2H), 7.51 (d, 2H), 4.15-4.09 (in, 2H), 3.62 (m, 2H), 2.93 (q, 2H), 2.80 (q, 2H), 2.75 (s, 2H), 2.63 (d, 3H), 1.44 (s, 9H), 1.25-1.18 (m, 6H).

### Example 160

### Preparation of 1-[4-({3,5-diethyl-1-[2-(methylamino)ethyl]-1H-pyrazol-4-yl}sulfonyl)phenyl]-3,3-dimethyl-2,5-pyrrolidinedione

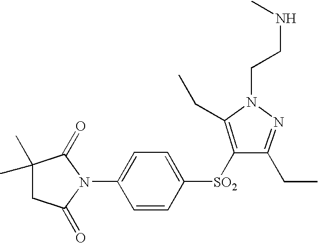

The compound was prepared using the same procedure described for Example 124. Product (0.32 g, 100%): MS (Electronspray) 447 (M+H)⁺, 2.85, Mp. 84-86° C., ¹H NMR (300 MHz, CD₃OD) δ 8.00 (d, 2H), 7.58 (d, 2H), 4.40 (t, 2H), 3.51 (t, 2H), 3.04 (q, 2H), 2.81 (q, 2H), 2.79 (s, 2H), 2.77 (s, 3H), 1.40 (s, 6H), 1.26-1.18 (m, 6H).

### Example 161

### Preparation of tert-butyl (1S)-2-{3,5-diethyl-4-[(4-fluorophenyl)sulfonyl-1H-pyrazol-1-yl}-1-methylethylcarbamate

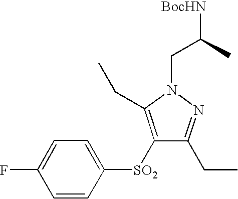

The compound was prepared using the same procedure described for Example 159. Product (0.202 g, 32%): MS (Electronspray) 440 (M+H)⁺, ¹H NMR (300 MHz, CDCl₃) δ 7.90-7.86 (m, 2H), 7.18-7.13 (m, 2H), 4.97 (m, 1H), 4.10-3.98 (m, 3H), 2.97 (q, 2H), 2.75 (q, 2H), 1.37 (s, 9H), 1.23-1.14 (m, 9H).

### Example 162

### Preparation of (1S)-2-{3,5-diethyl-4-[(4-fluorophenyl)sulfonyl]-1H-pyrazol-1-yl}-1-methylethylamine Bistrifluoroacetic Acid Salt

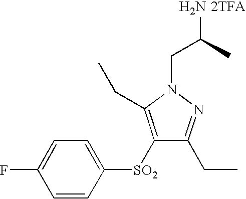

The compound was prepared using the same procedure described for Example 160. Product (0.08 g, 100%): ¹H NMR (300 MHz, CD₃OD) δ 7.97-7.93 (m, 2H), 7.34-7.29 (m, 2H), 4.33-4.19 (m, 2H), 3.87-3.81 (m, 1H), 3.10-2.97 (m, 2H), 2.80 (q, 2H), 1.31 (d, 3H), 1.23-1.14 (m, 6H).

### Example 163

### Preparation of tert-butyl (1S)-2-(4-{[4-(3,3-dimethyl-2,5-dioxo-1-Pyrrolidinyl)phenyl]sulfonyl}-3,5-diethyl-1H-pyrazol-1-yl)-1-methylethylcarbamate

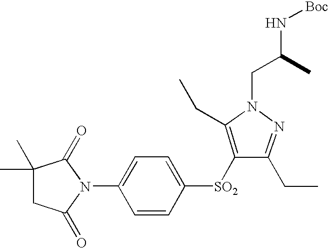

### Step 1: Preparation of 1-{4-[(3,5-diethyl-1H-pyrazol-4-yl)sulfonyl]phenyl}-3,3-dimethyl-2,5-pyrrolidinedione

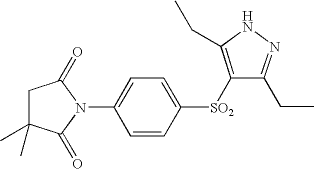

The compound was prepared using the same procedure as Example 127. To a solution of 4-[(3,5-diethyl-1H-pyrazol-4-yl)sulfonyl]aniline, (1.21 g, 3.19 mmol) and triethylamine (0.18 mL, 1.28 mmol) in pyridine (16 mL) and toluene (16 mL) was added 2,2-dimethylsuccinic anhydride (0.61 g, 4.78 mmol). The mixture was refluxed under argon for 16 h and concentrated under reduced pressure. The desired product (0.97 g, 78%) was isolated by MPLC with the elution of 50% EtOAc in Hexane. R=0.10 (50% EtOAc in Hexane); MS (Electronspray) 390 (M+H)⁺; ¹H NMR (300 MHz, CDCl₃) δ 7.97 (d, 2H), 7.52 (d, 2H), 2.96-2.88 (m, 4H), 1.34-1.25 (m, 6H).

### Step 2: Preparation of tert-butyl (1S)-2-(4-{[4-(3,3-dimethyl-2,5-dioxo-1-pyrrolidinyl)phenyl]sulfonyl}-3,5-diethyl-1H-pyrazol-1-yl)-1-methylethylcarbamate

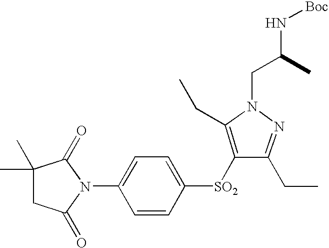

A solution of 2-[(tert-butoxycarbonyl)(methyl)amino]ethyl methanesulfonate (6.96 g, 27.47 mmol) in N,N-dimethylformamide (28 mL) was added to a suspension of the imide prepared in step 1 (2.14 g, 5.50 mmol) and cesium carbonate (10.74 g, 32.97 mmol) in N,N-dimethylformamide (40 mL). The mixture was stirred at 58° C. for 15 h under argon, then cooled to room temperature. Brine (50 mL) and ethyl acetate (30 mL) were added. The aqueous layer was separated and extracted with ethyl acetate (3×30 mL). Combined organic layers were dried over Na₂SO₄ and concentrated. The desired product was isolated by MPLC with the elution of 33% EtOAc in Hexane to give a white foamy solid (2.03 g, 68%)=0.24 (33% EtOAc in Hexane); MS (Electronspray) 548 (M+H)⁺; ¹H NMR (300 MHz, CDCl₃) δ 7.95 (d, 2H), 7.50 (d, 2H), 5.01 (s, 1H), 4.15-3.98 (m, 3H), 3.03-2.74 (m, 6H), 1.43 (s, 6H), 1.39 (s, 9H), 1.35-0.91 (m, 6H).

### Example 164

### Preparation of 1-[4-({1-[(2S)-2-aminopropyl]-3,5-diethyl-1H-pyrazol-4-yl}sulfonyl)phenyl]-3,3-dimethyl-2,5-pyrrolidinedione, Trifluoroacetic Acid Salt

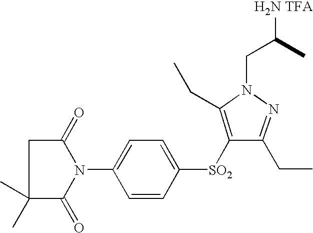

To a solution of Example 163 (0.17 g, 0.311 mmol) in dichloromethane (2 mL) was added trifluoroacetic acid (2 mL). The mixture was stirred for 2 h and concentrated under reduced pressure. The residue was triturated with ether and dried under vacuum to give a white solid (0.17 g, 100%). ¹H NMR (300 MHz, DMSO-d₆) δ 8.00 (d, 2H), 7.57 (d, 2H), 4.25 (dd, 2H), 3.81 (m, 1H), 3.03 (m, 2H), 2.83 (q, 2H), 2.78 (s, 2H), 1.39 (s, 6H), 1.32 (d, 3H), 1.26-1.16 (m, 6H).

### Example 165

### Preparation of tert-butyl (1S)-2-{3,5-diethyl-4-[(4-nitrophenyl)sulfonyl]-1H-pyrazol-1-yl}-1-methylethylcarbamate

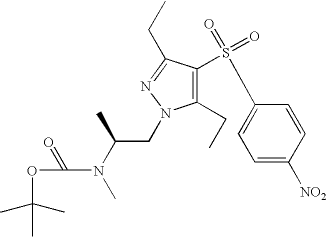

The compound was prepared using the same procedure described for Example 128. Product (0.90 g, 47%): R=0.53 (50% EtOAc in Hexane), MS (Electronspray) 467 (M+H)⁺, ¹H NMR (300 MHz, CDCl₃) δ 8.34-8.31 (m, 2H), 8.06-8.02 (m, 2H), 4.87 (m, 1H), 4.13-4.00 (m, 3H), 3.00 (q, 2H), 2.79-2.71 (m, 2H), 1.35 (s, 9H), 1.24-1.16 (m, 9H).

### Example 166

### Preparation of tert-butyl (1S)-2-{4-[(4-aminophenyl)sulfonyl]-3,5-diethyl-1H-pyrazol-1-yl}-1-methylethylcarbamate

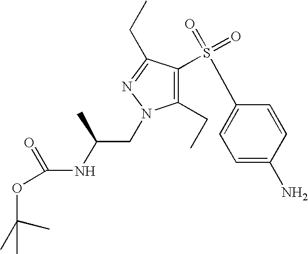

The compound was prepared using the same procedure described for Example 129. Product (0.43 g, used without further purification). R=0.20 (50% EtOAc in Hexane), MS (Electronspray) 437 (M+H)⁺, ¹H NMR (300 MHz, CDCl₃) δ 7.63 (d, 2H), 6.64 (d, 2H), 5.08 (m, 1H), 4.13-3.96 (m, 3H), 3.03-2.92 (m, 2H), 2.7 (q, 2H), 1.39 (s, 9H), 1.22-1.12 (m, 9H).

### Example 167

### Preparation of N-{(1S)-2-[4-({4-[(2,2-dimethylpropanoyl)amino]phenyl}sulfonyl)-3,5-diethyl-1H-pyrazol-1-yl]-1-methylethyl}-2,2-dimethylpropanamide

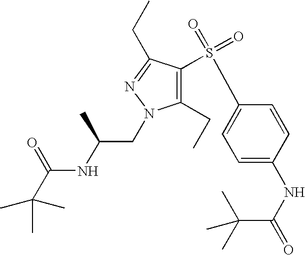

The compound was prepared using the same procedure described for Example 117. Product (0.115 g, 60%): MS (Electronspray), ¹H NMR (300 MHz, CDCl₃) δ 7.77 (d, 2H), 7.65 (d, 2H), 5.05 (m, 1H), 4.10-3.96 (m, 3H), 3.00-2.91 (m, 2H), 2.75 (q, 2H), 1.39 (s, 9H), 1.31 (s, 9H), 1.22-1.11 (m, 9H).

### Example 168

### Preparation of N-[4-[{1-[(2S)-2-aminopropyl]-3,5-diethyl-1H-pyrazol-4-yl}sulfonyl)phenyl]-2,2-dimethylpropanamide

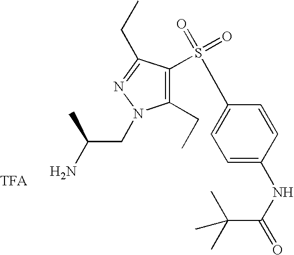

The compound was prepared using the same procedure described for Example 124. Product (0.143 g, '100%): ¹H NM (300 MHz, CD₃OD) δ 7.66 (d, 2H), 7.60 (d, 2H), 4.08 (m, 2H), 3.80 (m, 1H), 2.85 (q, 2H), 2.67 (q, 2H), 1.26 (d, 3H), 1.19 (s, 9H), 1.06-0.98 (m, 6H).

### Example 169

### Preparation of tert-butyl (1S)-2-(3,5-diethyl-4-{[4-(2-oxo-1-pyrrolidinyl)phenyl]sulfonyl}-1H-pyrazol-1-yl)-1-methylethylcarbamate

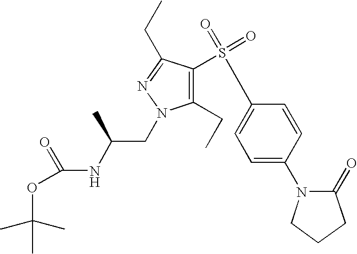

5-Bromovaleryl chloride (0.198 g, 0.962 mmol) was added to a solution of Example 146 (0.4 g, 0.916 mmol) and pyridine (0.148 mL, 1.83 mmol) in dichloromethane (4.6 mL) at room temperature. The mixture was stirred for 3 h and concentrated. The residue was taken up in ethyl acetate (10 mL) and filtered through a plug of silica gel and the filtrate concentrated. The concentrate was dissolved in N,N-dimethylformamide (7.8 mL) and potassium carbonate (0.43 g, 3.12 mmol) was added and the mixture stirred at room temperature for 16 h. The mixture was diluted with ethyl acetate (50 mL) and water (10 mL). The organic layer was isolated and dried over MgSO₄ and concentrated. The product (0.33 g, 81%) was isolated by column chromatography (50% EtOAc in Hexane). R_{f}=0.11 (50% EtOAc in Hexane), MS (Electronspray) 519 (M+H)⁺, ¹H NMR (300 MHz, CDCl₃) δ 7.87-7.83 (m, 2H), 7.42-7.38 (m, 2H), 5.06 (m, 1H), 4.11-3.96 (m, 3H), 3.66 (m, 2H), 2.97-2.94 (m, 2H), 2.82-2.73 (m, 2H), 2.56 (m, 2H), 1.94 (m, 4H), 1.39 (s, 9H), 1.24-1.14 (m, 9H).

### Example 170

### Preparation of 1-[4-({1-[(2S)-2-aminopropyl]-3-ethyl-5-methyl-1H-pyrazol-4-yl}sulfonyl)phenyl]-2-pyrrolidinone

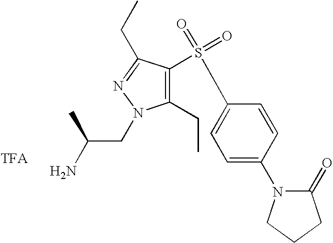

The compound was prepared using the same procedure described for Example 124. Product (0.311 g, 100%): ¹H NMR (300 MHz, CDCl₃) δ 7.92 (d, 2H), 7.51 (d, 2H), 4.33-4.18 (m, 2H), 3.83 (m, 1H), 3.70 (m, 2H), 3.08-2.99 (m, 2H), 2.81 (q, 2H), 2.54 (t, 2H), 1.97 (m, 4H), 1.32 (d, 3H), 1.25-1.17 (m, 6H).

### Example 171

### Preparation of tert-butyl (1S)-2-[4-({4-[(3-chloro-2,2-dimethylpropanoyl)amino]phenyl}sulfonyl)-3,5-diethyl-1H-pyrazol-1-yl]-1-methylethylcarbamate

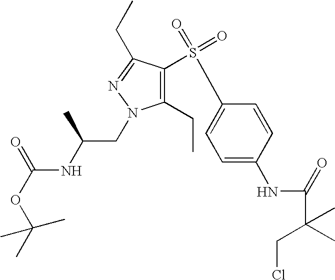

3-Chloropivolyl chloride (0.152 g, 0.962 mmol) was added to a solution of Example 146 (0.4 g, 0.916 mmol) and pyridine (0.148 mL, 1.83 mmol) in dichloromethane (4.6 mL) at room temperature. The mixture was stirred for 3 h and concentrated. The residue was taken up in ethyl acetate (10 mL) and filtered through a plug of silica gel and the filtrate concentrated to give 0.455 g, 89% of the product. R_{f}=0.23 (50% EtOAc in Hexane), MS (Electronspray) 557 (M+2)⁺, ¹H NMR (300 MHz, CDCl₃) δ 7.80 (d, 2H), 7.66 (d, 2H), 5.02 (m, 1H), 4.09-3.97 (m, 3H), 3.00-2.93 (m, 2H), 2.80-2.72 (m, 2H), 1.42-1.38 (m, 15H1), 1.23-1.12 (m, 9H).

### Example 172

### Preparation of tert-butyl (1S)-2-(4-{[4-(3,3-dimethyl-2-oxo-1-azetidinyl)phenyl]sulfonyl}-3,5-diethyl-1H-pyrazol-1-yl)-1-methylethylcarbamate

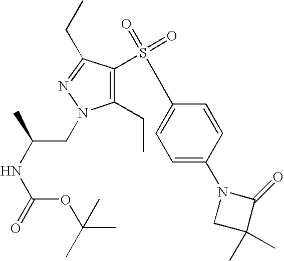

A solution of Example 171 (0.455 g, 0.820 mmol) in N,N-dimethylformamide (8.2 mL) was treated with potassium carbonate (0.453 g, 3.28 mmol) and the mixture stirred at room temperature for 16 h. The mixture was diluted with ethyl acetate (50 mL) and water (10 mL). The organic layer was isolated and dried over MgSO₄ and concentrated. The product (0.43 g, 100%) was isolated by column chromatography (50% EtOAc in Hexane). R_{f}=0.23 (50% EtOAc in Hexane), MS (Electronspray) 519 (M+H)⁺, ¹H NMR (300 M, CDCl₃) δ 7.80-7.75 (m, 2H), 7.38-7.34 (m, 2H), 5.05 (m, 1H), 4.09-3.93 (m, 31H), 2.94-2.89 (m, 2H), 2.75-2.68 (m, 2H), 1.36-1.32 (m, 15H), 1.23-1.09 (In 9H).

### Example 173

### Preparation of 1-[4-({1-[(2S)-2-aminopropyl]-3,5-diethyl-1H-pyrazol-4-yl}sulfonyl)phenyl]-3,3-dimethyl-2-azetidinone Hydrochloride

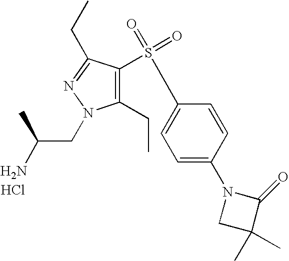

The compound was prepared using the same procedure described for Example 124. Product (0.402 g, 100%): ¹H NMR (300 MHz, CD₃OD) δ 7.86 (d, 2H), 7.51 (d, 2H), 4.34-4.19 (m, 2H), 3.82 (m, 1H), 3.57 (s, 2H), 3.11-2.93 (m, 2H), 2.82-2.75 (m, 2H), 1.37-1.17 (m, 15H).

### Example 174

### Preparation of 1-(2-iodoethyl)cyclopentanecarboxylic Acid

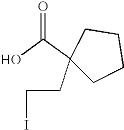

A solution of 2-oxa-spiro[4,4]decan-1-one (1 g, 9.60 mmol) and trimethylsilyl iodide (2.05 g , 14.4 mmol) in dichloromethane (14.3 mL) was refluxed for 3 h, cooled to room temperature and quenched with water (10 mL) and diluted with dichloromethane (50 mL). The organic layer was isolated, dried over MgSO₄ and concentrated to give a dark yellow solid (1.81 g, 95%, used in the next step without further purification).

### Example 175

### Preparation of tert-butyl (1S)-2-(3,5-diethyl-4-{[4-(1-oxo-2-azaspiro[4,4]non-2-yl)phenyl]sulfonyl}-1H-pyrazol-1-yl)-1-methylethylcarbamate

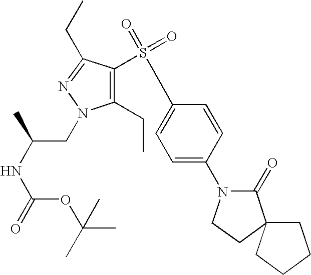

Oxalyl Chloride (0.37 mL, 4.23 mmol) was added to a cooled solution of Example 174 (1 g, 3.73 mmol) and a drop of N,N-dimethylformamide. The mixture was stirred at 0° C. for 15 min, concentrated and dissolved in dichloromethane (1.5 mL). The resulting solution was added to a solution of Example 146 and triethylamine (1.04 mL) in dichloromethane (1.5 mL), and the mixture stirred at room temperature for 30 min and concentrated. The residue was dissolved in N,N-dimethylformamide (10 mL) treated with potassium carbonate (1.38 g, 9.99 mmol). The product (0.097 g) was isolated by HPLC. MS (Electronspray) 574 (M+H)⁺, ¹H NMR (300 MHz, CDCl₃) δ 7.86-7.79 (m, 4H), 5.05 (m, 1H), 4.10-3.97 (m, 3H), 3.76 (t, 2H), 3.02-2.96 (m, 2H), 2.77 (q, 2H), 2.10-2.04 (m, 2H), 1.75-1.33 (m, 19H), 1.23-1.11 (m, 9H).

### Example 176

### Preparation of 2-[4-({1-[(2S)-2-aminopropyl]-3,5-diethyl-1H-pyrazol-4-yl}sulfonyl)phenyl]-2-azaspiro[4,4]nonan-1-one

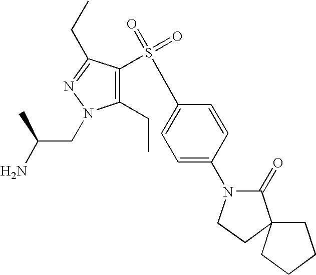

The product was obtained by treatment of Example 175 with anhyd HCl in ether: (0.072 g, 100%): ¹H NMR (300 MHz, CD₃OD) δ 7.85 (s, 4H), 4.30-4.17 (m, 2H), 3.86-3.81 (m, 3H), 3.06-2.96 (m, 2H), 2.78 (q, 2H), 2.13 (t, 2H), 1.75-1.14 (m, 19H).

### Example 177

### Preparation of tert-butyl (1S)-2-(3,5-diethyl-4-{[4-(1-oxo-1,3-dihydro-2H-isoindol-2-yl)phenyl]sulfonyl}-1H-pyrazol-1-yl)-1-methylethylcarbamate

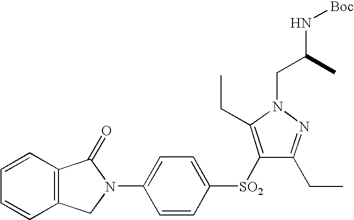

### Step 1. Preparation of 2-(iodomethyl)benzoic Acid

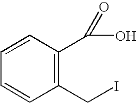

The compound was prepared using the same procedure described for Example 174. Product (3.7 g, 92%): MS (Electronspray) 134 (M−128)⁺. ¹H NMR (300 MHz, CDCl₃) δ 7.84 (d, 1H), 7.31-7.16 (m, 3H), 4.84 (s, 2H).

### Step 2. Preparation of tert-butyl (1S)-2-(3,5-diethyl-4-{[4-(1-oxo-1,3-dihydro-2H-isoindol-2-yl)phenyl]sulfonyl}-1H-pyrazol-1-yl)-1-methylethylcarbamate

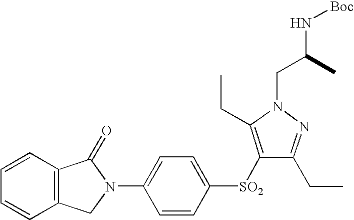

The compound was prepared from the product of step 1, using the same procedure described for Example 176. Product: MS (Electronspray) 553 (M+H)⁺. ¹H NMR (300 MHz, CDCl₃) δ 8.07-7.50 (m, 8H), 5.05 (m, 1H), 4.89 (s, 2H), 4.13-3.99 (m, 3H), 3.03 (q, 2H), 2.80 (q, 2H), 1.39 (s, 9H), 1.26-1.14 (m, 9H).

### Example 178

### Preparation of 2-[4-({1-[(2S)-2-aminopropyl]-3,5-diethyl-1H-pyrazol-4-yl}sulfonyl)phenyl]-1-isoindolinone

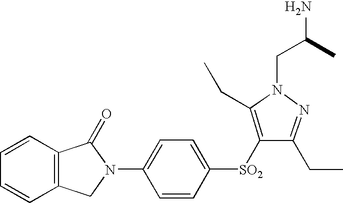

The compound was prepared using the same procedure described for Example 148. Product: MS (Electronspray) 453 (M+H)⁺, RT=2.18. ¹H NMR (300 MHz, DMSO) δ 8.14-7.51 (m, 8H), 4.18 (dd, 2H), 3.65-3.61 (m, 1H), 3.34 (s, 2H), 2.96 (q, 2H), 2.72 (q, 2H), 1.18-1.06 (m, 9H).

### Example 179

### Preparation of 2-[4-({1-[(2S)-2-aminopropyl]-3,5-diethyl-1H-pyrazol-4-yl}sulfonyl)phenyl]-1-isoindolinone

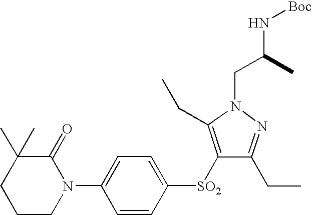

### Step 1. Preparation of 4-methyl-1,4-pentanediol

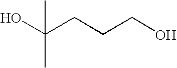

γ-Butyrolactone (5 mL, 0.065 mol) was added dropwise to a solution of methyl magnesium bromide (87, 0.260 mol, 3 M) in ether (5 mL) an ice bath over 15 min. The mixture was heated on an oil bath at 45° C. for 2 h. The mixture was quenched with water (5 mL) concentrated and the residue taken up in ethyl acetate (50 mL) and dried over Na₂SO₄ and concentrated to give a colorless viscous oil (5.56 g, 72%). MS (Electronspray) 119 (M+H)⁺. ¹H NMR (300 MHz, CDCl₃) δ 3.69-3.64 (m, 2H), 2.13 (s, 2H), 1.71-1.56 (m, 4H), 1.24 (s, 6H).

### Step 2. Preparation of 3,3-dimethyltetrahydro-2H-pyran-2-one

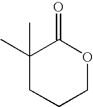

A mixture of the product of step 1 (3.26 g, 0.0276 mol) and formic (11 mL) was added to sulfuric acid (116 mL) in a water bath (17-20° C.) over 1 h. The mixture was stirred for 1.3 h and poured into ice and extracted with ether (3×30 mL) and concentrated to give 0.96 g (27%) of a colorless oil. MS (Electronspray) 128 (M)⁺. ¹H NMR (300 MHz, CDCl₃) δ 4.34 (t, 2H), 1.94-1.73 (m, 4H), 1.30 (s, 6H).

### Step 3. Preparation of 5-iodo-2,2-dimethylpentanoic Acid

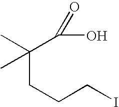

The compound was prepared from the product of step 2 and Example 146 using the same procedure described for Example 174. Product (1.85 g, 96%): GC/MS 257 (M+H)⁺. ¹H NMR (300 MHz, CDCl₃) δ 3.18 (q, 2H), 1.89-1.62 (m, 4H), 1.24 (s, 6H).

### Step 4. Preparation of tert-butyl (1S)-2-(4-{[4-(3,3-dimethyl-2,5-dioxo-1-pyrrolidinyl)phenyl]sulfonyl}-3,5-diethyl-1H-pyrazol-1-yl)-1-methylethylcarbamate

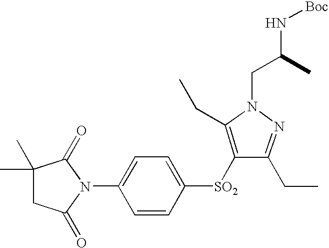

The compound was prepared from the compound from step 3 and using the same procedure described for Example 176. Product (0.911 g, 73%): R_{f}=0.17 (50% EtOAc in Hexanes), MS (Electronspray) 547 (M+H)⁺. ¹H NMR (300 MHz, CDCl₃) δ 7.85 (d, 2H), 7.37 (d, 2H), 5.07 (m, 1H), 4.13-3.97 (m, 3H), 3.67 (t, 2H), 3.02-2.93 (m, 2H), 2.77 (q, 2H), 2.05-1.81 (m, 4H), 1.42 (s, 9H), 1.31 (s, 6H), 1.26-1.13 (m, 9H).

### Example 180

### Preparation of 1-[4-({1-[(2S)-2-aminopropyl]-3,5-diethyl-1H-pyrazol-4-yl}sulfonyl)phenyl]-3,3-dimethyl-2-piperidinone Hydrochloride

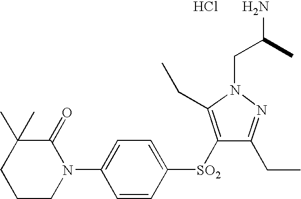

The compound was prepared using the same procedure described for Example 124 Product (0.799 g, 93%): MS (Electronspray) 447 (M+H)⁺, RT=2.07, Mp. 131° C. ¹H NMR (300 MHz, CDCl₃) δ 7.83 (d, 2H), 7.46 (d, 2H), 4.28-4.10 (m, 3H), 3.63 (t, 21I), 2.96 (q, 2H), 2.71 (q, 2H), 1.93-1.73 (m, 4H), 1.18-1.05 (m, 15H).

### Example 181

### Preparation of tert-butyl (1S)-2-{3,5-diethyl-4-[(3-nitrophenyl)sulfanyl]-1H-pyrazol-1-yl}-1-methylethylcarbamate

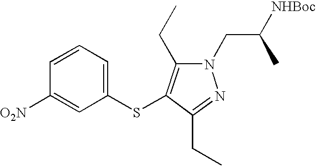

### Step 1. Preparation of 1-(aminosulfanyl)-3-nitrobenzene

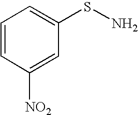

To a round bottom flask was added silver nitrate (2.8 g, 16.48 mmol) dissolved in methanol at 0° C. and was added the disulfide (5.0 g, 16.22). Ammonia was then bubbled through over a period of 1 hour and at room temperature for 1.5 hours. The salts were then filtered off and the suspension was concentrated down. The residue was then taken up into ether and filtered. The filtrate was then washed with water. The organic phase was dried under anhydrous magnesium sulfate and concentrated under reduced pressure to yield the desired product (2.69 g, 97% yield).

### Step 2. Preparation of 4-[(3-nitrophenyl)sulfanyl]-3,5-heptanedione

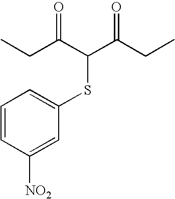

To a round bottom flask was added the compound of step 1 (2.69 g, 15.81 mmol) dissolved in ethanol. Ammonium chloride (2.58 g, 48.23 mmol) and 3,5-heptadione (11 mL, 82.05 mmol) were then added and were set to stir for 20 hours. The solvent was removed and the residue was taken up into ether. The organic phase was washed with water, dried under anhydrous magnesium sulfate and concentrated under reduced pressure to yield the desired product.

### Step 3. Preparation of tert-butyl (1S)-2-{3,5-diethyl-4-[(3-nitrophenyl)sulfanyl]-1H-pyrazol-1-yl}-1-methylethylcarbamate

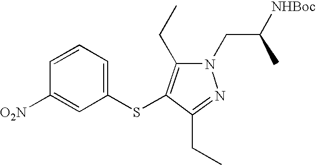

To a round bottom equipped with a condenser under argon was added the product of step 2 (1.07 g, 4.2 mmol) dissolved in ethanol (10 mL) and amine (1.2 g, 6.3 mmol) dissolved in ethanol (15 mL). The reaction mixture was allowed to stir for 10 minutes after which acetic acid (5 drops) was added to the mixture and was then heated to 90° C. for 1.5 hours. The reaction was cooled to room temperature and used in the next step without further purification (1.9 g).

### Example 182

### Preparation of tert-butyl (1S)-2-{3,5-diethyl-4-[(3-nitrophenyl)sulfonyl]-1H-pyrazol-1-yl}-1-methylethylcarbamate

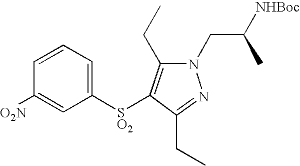

To a round bottom under argon was added Example 181 (1.9 g, 4.67 mmol) dissolved in dichloromethane. To this solution was added MCPBA (2.42 g, 14 mmol) and was set to stir for 18 hours. Saturated sodium thiosulfate (35 mL), saturated sodium bicarbonate (70 mL), and dichloromethane (50 mL) were added to the reaction mixture to stir for 0.5 hours. Water was then added and the product was extracted with dichloromethane (3×50 mL). It was then dried over anhydrous sodium sulfate and concentrated under reduced pressure. The product was used without further purification (79 g, 97%).

### Example 183

### Preparation of tert-butyl (1S)-2-{4-[(3-aminophenyl)sulfonyl]-3,5-diethyl-1H-pyrazol-1-yl}-1-methylethylcarbamate

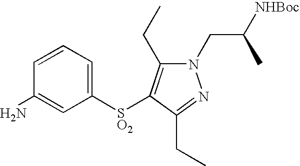

To a round bottom was added Raney Nickel catalyst and was washed twice with ethanol (50 mL). An additional 200 mL of ethanol was added to the flask and was set to stir for 5 minutes. Example 182 (3.02 g, 6.48 mmol) dissolved in ethanol was added to the solution. The flask was then evacuated by vacuum and equipped with a hydrogen balloon. The mixture was allowed to stir for 3.5 hours under hydrogen. The reaction mixture was then filtered through celite and washed with ethanol (500 mL) then ethyl acetate (200 mL). Evaporation of the solvent gave the crude product, which was then purified by MPLC using a 1:1 mixture of ethyl acetate and hexane.

### Example 184

### Preparation of tert-butyl (1S)-2-(4-{[3-(acetylamino)phenyl]sulfonyl}-3,5-diethyl-1H-pyrazol-1-yl)-1-methylethylcarbamate

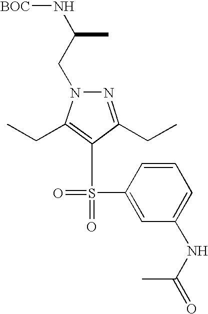

To a round bottom flask under argon was added Example 183 (300 mg, 0.687 mmol) in dry dichloromethane. To this was added the pyridine polymer (220 mg, 2.06 mmol) via the top of the flask. This was left to stir for 10 minutes at room temperature prior to the careful dropwise addition of t-butylacetyl chloride through the top of the flask. This was left to stir at room temperature for 18 hours. Approximately 20 mL of dichloromethane was carefully introduced into the flask. The mixture was then filtered through celite and washed with dichloromethane (2×50 mL). This was then purified by MPLC (Biotage using a 1:1 mixture of ethyl acetate and hexane) to give the desired product.

### Example 185

### Preparation of N-[3-({1-[(2S)-2-aminopropyl]-3,5-diethyl-1H-pyrazol-4-yl}sulfonyl)phenyl]-2,2-dimethylpropanamide

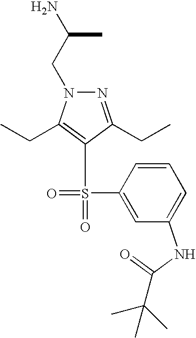

Example 184 (180 mg) dissolved in dichloromethane was added a solution of 2.0 M HCl in ether. The mixture was set to stir for 18 hours. The mixture was then concentrated under reduced pressure to yield a pure crystal product.

### Example 186

### Preparation of tert-butyl (1S)-2-{3,5-diethyl-4-[(3-{[(1-methylcyclopropyl)carbonyl]amino}phenyl)sulfonyl]-1H-pyrazol-1-yl}-1-methylethylcarbamate

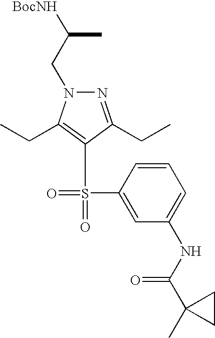

To a solution of 1-methylcyclopropane carboxylic acid (80 mg, 0.80 mmol) in N,N-dimethylformamide (6 mL) at 0° C. was added N′-(3-dimethylaminopropyl)-N-ethyl carbodiimide (215 mg, 1.12 mmol), 1-Hydroxy Benzotriazole hydrate (216 mg, 1.6 mmol) and Example 183 (350 mg, 0.80 mmol) in 4 mL of N,N-dimethylformamide. The resulting solution was heated to reflux for 6 hours before it was cooled to room temperature. Water was then added and the mixture was extracted with diethyl ether twice. The combined organic layers were washed with water, dried over anhydrous sodium sulfate and concentrated under reduced pressure. The resulting residue was purified with flash chromatography (Biotage flash 40M) using 1:1 Hexane:ethyl acetate to afford 1 (130 mg, 32%). MS (Electronspray) 519 (M+H)⁺; ¹H NMR (300 MHz, CDCl₃) 0.95 (q, 2H), 1.16 (m, 6H), 1.22 (m, 2H), 1.36 (s, 9H), 1.44 (m, 6H), 2.71 (q, 2H), 2.94 (q, 2H), 4.00 (m, 3H), 5.23 (s, 1H), 7.25 (t, 1H), 7.45 (d, 2H), 7.58 (d, 1H), 7.85 (s, 1H).

### Example 187

### Preparation of N-[3-({1-[(2S)-2-aminopropyl]-3,5-diethyl-1H-pyrazol-4-yl}sulfonyl)phenyl]-1-methylcyclopropanecarboxamide Dihydrochloride

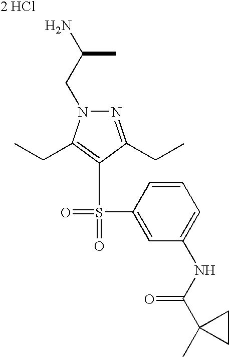

To a solution of Example 186 (130 mg, 0.25 mmol) in dichloromethane (2.5 mL) was added hydrochloric acid (2.0M in ether, 2.5 mL). The mixture was then stirred at room temperature for 15 hours, then concentrated in vacuo. The resulting residue was triturated with diethyl ether to obtain the product as white solid (85 mg, 70%). MS (Electronspray) 419 (M+H)⁺; ¹H NMR (300 MHz, d₆-DMSO) 0.63 (q, 2H), 1.08 (m, 11H), 1.39 (s, 3H), 2.70 (q, 2H), 2.92 (q, 2H), 3.74 (m, 1H), 4.16 (m, 2H), 7.49 (d, 2H), 7.87 (m, 1H), 8.12 (s, 1H), 8.31 (s, 1H), 9.53 (s, 1H).

### Example 188

### Preparation of tert-butyl (1S)-2-(4-{[3-(4,4-dimethyl-2,6-dioxo-1-piperidinyl)phenyl]sulfonyl}-3,5-diethyl-1H-pyrazol-1-yl)-1-methylethylcarbamate

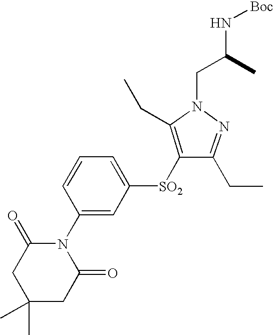

To a solution of Example 183 (350 mg, 0.86 mmol) dissolved in Tetrahydrofuran under argon was added anhydride (154 mg, 1.08 mmol). p-Toluenesulfonic acid monohydrate (23 mg, 0.12 mmol) was then added to the reaction mixture and was heated to reflux for 18 hours. CDI (180 mg, 1.11 mmol) was added and was stirred for an additional 24 hours. The residue was absorbed onto silica gel and was purified by MPLC (Biotage Flash 12M) using 3:1 Hexane and ethyl acetate (280 mg).

### Example 189

### Preparation of 1-[3-({1-[(2S)-2-aminopropyl]-3,5-diethyl-1H-pyrazol-4-yl}sulfonyl)phenyl]-4,4-dimethyl-2,6-piperidinedione Hydrochloride

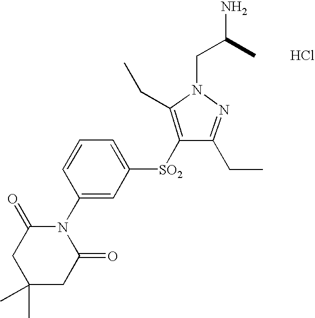 2M HCl solution in ethyl ether was added to a solution of Example 188 (190 mg, 0.35 mmol) was dissolved in dichloromethane at room temperature and the reaction mixture was then stirred for 48 hours. Removed solvent under reduced pressure (159 mg).

### Example 190

### Preparation of tert-butyl (1S)-2-{4-[(4-amino-3-chlorophenyl)sulfonyl]-3,5-diethyl-1H-pyrazol-1-yl}-1-methylethylcarbamate

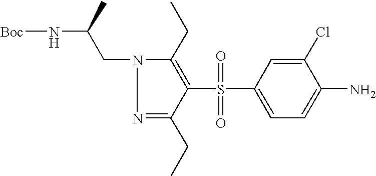

To a round bottom flask under argon was placed Example 146 (200 mg, 0.458 mmol) in dichloromethane (5 mL). This was cooled to 0° C. in an ice bath for 15 minutes. To this mixture was added acetic acid (0.8 mL) dropwise. After stirring for 5 minutes Chloramine T (115 mg, 0.504 mmol) was added in the same manner. This was left to come slowly to room temperature and the reaction was monitored for loss of starting material. Next 20 mL of dichloromethane was added followed by 50 mL of saturated sodium bicarbonate. The mixture was washed with an additional 50 mL of saturated sodium bicarbonate and water (2×50 mL). The organic layer was then dried over anhydrous magnesium sulfate. The crude product was chromatographed using MPLC (Biotage using a mixture of 1:1 ethyl acetate hexane) to yield the desired compound (130 mg, 60% yield).

### Example 191

### Preparation of tert-butyl (1S)-2-[4-({3-chloro-4-[(2,2-dimethylpropanoyl)amino]phenyl}sulfonyl)-3,5-diethyl-1H-pyrazol-1-yl]-1-methylethylcarbamate

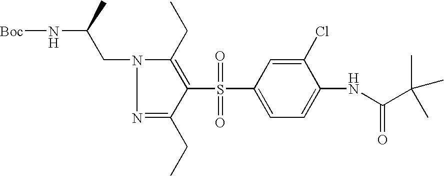

To a dry round bottom flask under argon was added Example 190 (125 mg, 0.266 mmol) dissolved in THF (8 mL). The mixture was stirred at −78C for 10 minutes prior to the dropwise addition of sec-butyl lithium (0.408 mL, 0.530 mmol). The reaction mixture was left to stir at −78C for 1 hour. A 1M solution of t-butyl carbonyl chloride (0.266 mL, 0.266 mmol) was added dropwise to the reaction mixture and was stirred for an additional hour. The reaction mixture was then quenched with water and extracted with ether (50 mL). The organic layer was then washed with saturated sodium bicarbonate and water. The organic layers were then combined and dried over anhydrous magnesium sulfate and concentrated under reduced pressure. The crude product was then purified using a mixture of 1:1 ethyl acetate to give the desired product (25 mg, 17% yield).

### Example 192

### Preparation of N-[4-({1-[(2S)-2-aminopropyl]-3,5-diethyl-1H-pyrazol-4-yl}sulfonyl)-2-chlorophenyl]-2,2-dimethylpropanamide, Trifluoroacetic Acid Salt

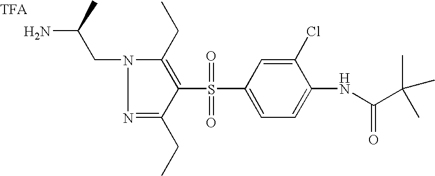

To a solution of Example 191 (64 mg, 0.14 mmol) dissolved in dichloromethane at room temperature was added dropwise the acid chloride followed by the polymer-bound pyridine (42 mg, 0.38 mmol). The reaction mixture was stirred for 18 hours and then concentrated under reduced pressure. The crude product was then purified by MPLC (Biotage, 12M using a 3:1 mixture of hexane and ethyl acetate) to give the desired product (23 mg, 30% yield).

### Example 193

### Preparation of tert-butyl (1S)-2-{4-[(4-amino-3-bromophenyl)sulfonyl]-3,5-diethyl-1H-pyrazol-1-yl}-1-methylethylcarbamate

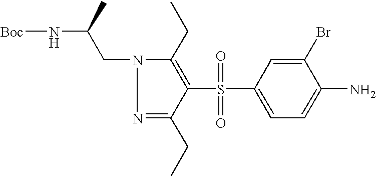

In a dry round bottom flask under argon was added Example 146 (50 mg, 0.114 mmol)) in dichloromethane (3 mL). To this was added acetic acid (0.5 mL) and NBS (13 mg, 0.103 mmol). The reaction mixture was then set to stir for 75 minutes at room temperature. The reaction mixture was then extracted with dichloromethane and washed with saturated sodium bicarbonate and water. The organic layer was then dried over anhydrous magnesium sulfate and concentrated under reduced pressure. The crude product was purified by MPLC (Biotage) using a 1:1 mixture of ethyl acetate and hexane (38 mg, 66% yield).

### Example 194

### Preparation of tert-butyl (1S)-2-[4-({3-bromo-4-[(2,2-dimethylpropanoyl)amino]phenyl}sulfonyl)-3,5-diethyl-1H-pyrazol-1-yl]-1-methylethylcarbamate

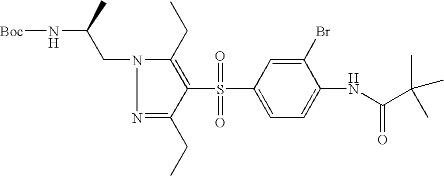

To a round bottom flask under argon was added Example 193 (52 mg, 0.101 mmol) in dry THF. This solution was cooled to −78C and sec-butyl lithium (0.153 mL, 0.200 mmol) was added and stirred for 1 hour. To this was added dropwise the acid chloride (0.13 mL, 0.101 mmol) through the top of the flask. This was left to stir at −78° C. for 1 hour. The reaction was quenched with water and extracted with ether (50 mL). This layer was then washed with saturated sodium bicarbonate (2×50 mL), and water (2×50 mL). The organic layer was then dried under anhydrous magnesium sulfate and concentrated under reduced pressure. The crude product was purified by MPLC (Biotage) using a 1:1 mixture of ethyl acetate and hexane (0.18 mg, 30%).

### Example 195

### Preparation of N-[4-({1-[(2S)-2-aminopropyl]-3,5-diethyl-1H-pyrazol-4-yl}sulfonyl)-2-bromophenyl]-2,2-dimethylpropanamide Hydrochloride

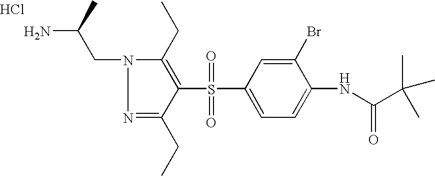

Example 194 (44 mg, 0.0735 mmol) dissolved in dichloromethane was added a solution of 2.0 M HCl in ether. The mixture was set to stir for 18 hours. The mixture was then concentrated under reduced pressure to yield a pure crystal product (20 mg, 51%).

### Example 196

### Preparation of tert-butyl (1S)-2-{3,5-diethyl-4-[(4-fluorophenyl)sulfonyl]-1H-pyrazol-1-yl}-1-methylethyl(methyl)carbamate

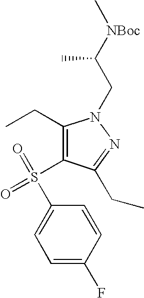

### Step 1. Preparation of (2S)-2-[(tert-butoxycarbonyl)(methyl)amino]propyl tert-butyl Carbonate

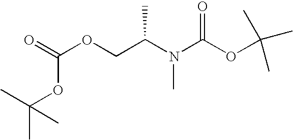

Lithium aluminum hydride (34.24 mL, 0.0342 mol) was added dropwise to a solution of (2-hydroxy-1-methyl-ethyl)-carbamic acid t-butyl ester (2 g, 0.0114 mol) in tetrahydrofuran (55 mL) and the mixture refluxed for 20 h. The reaction mixture was cooled to room temperature, quenched with water (5 mL) and saturated sodium bicarbonate (10 mL). The mixture was extracted with ether (3×50 mL) and the ether extract dried over MgSO₄ and concentrated. The residue was treated with di-tert-butyl dicarbonate (2.49 g, 0.0114 mol) and the product (1 g, 30%) isolated by column chromatography (50% EtOAc in Hexane). MS (Electronspray) 289 (M+H)⁺, ¹H NMR (300 MHz, CDCl₃) δ 4.04 (m, 2H), 2.76 (s, 3H), 1.48 (s, 9H), 1.47 (s, 9H), 1.14 (d, 3H).

### Step 2. Preparation of tert-butyl (1S)-2-hydroxy-1-methylethyl(methyl)carbamate

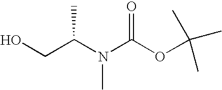

Potassium hydroxide (0.21 g, 3.460 mmol) was added to a solution of the product of step 1 in methanol/water (3/0.2 mL). The mixture was stirred for 1.5 h and diluted with ether (10 mL) and stirring continued for 16 h. The mixture was dried over MgSO₄ and concentrated. The product (0.4 g, 61%) isolated by column chromatography (33% EtOAc in Hexane). R_{f}=0.37 (33% EtOAc in Hexane), MS (Electronspray) 190 (M+H)⁺, ¹H NMR (300 MHz, CDCl₃) δ 4.47-4.29 (m, 1H), 4.05 (m, 2H), 2.70 (s, 3H), 1.41 (s, 9H), 1.10 (d, 3H).

### Step 3. Preparation of tert-butyl (1S)-2-{3,5-diethyl-4-[(4-fluorophenyl)sulfonyl]-1H-pyrazol-1-yl}-1-methylethyl(methyl)carbamate

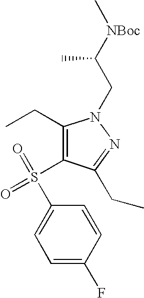

The compound was prepared from the compound of step 2, using the same procedure described for Example 128. Product (0.013 g): MS (Electronspray) 454 (M+H)⁺, ¹H NMR (300 MHz, CDCl₃) δ 7.85 (d, 2H), 7.12 (d, 2H), 4.33-3.88 (m, 3H), 2.95-2.61 (m, 7H), 1.29 (s, 9H), 1.20-1.14 (m, 9H).

### Example 197

### Preparation of N-((1S)-2-{3,5-diethyl-4-[(4-fluorophenyl)sulfonyl-1H-pyrazol-1-yl}-1-methylethyl)-N-methylamine, Trifluoroacetic Acid Salt

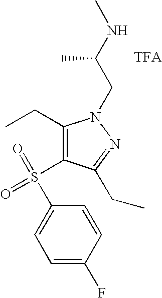

The compound was prepared using the same procedure described for Example 130. Product (0.013 g): ¹H NMR (300 MHz, CD₃OD) δ 7.98-7.93 (m, 2H), 7.35-7.29 (m, 2H), 4.41-4.28 (m, 2H), 3.79-3.73 (m, 1H), 3.12-2.91 (m, 2H), 2.88-2.75 (m, 5H), 1.29 (d, 3H), 1.24-1.15 (m, 6H).

### Example 198

### Preparation of 1-[4-({1-[2-(dimethylamino)ethyl]-3,5-diethyl-1H-pyrazol-4-yl}sulfonyl)phenyl]-3,3-dimethyl-2,5-pyrrolidinedione

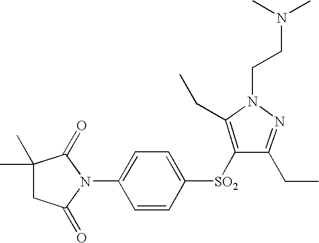

The compound was prepared using the same procedure described for Example 130. Product (0.18 g, 51%): MS (Electronspray) 461 (M+H)⁺, RT=2.82, ¹H NMR (300 MHz, CDCl₃) δ 7.96 (d, 2H), 7.50 (d, 2H), 4.12 (t, 2H).

### Example 199

### Preparation of tert-butyl (1R)-2-(4-{[4-(3,3-dimethyl-2,5-dioxo-1-pyrrolidinyl)phenyl]sulfonyl}-3,5-diethyl-1H-pyrazol-1-yl)-1-methylethylcarbamate

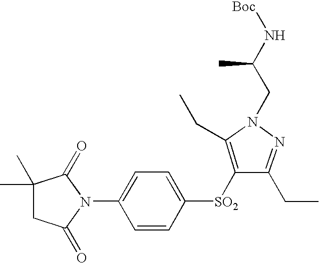

The compound was prepared using the same procedure described for Example 128. Product (0.18 g, 43%): R_{f}=0.24 (50% EtOAc in Hexane), MS (Electronspray) 548 (M+H)⁺, ¹H NMR (300 MHz, CDCl₃) δ 7.95 (d, 2H), 7.50 (d, 2H), 5.01 (s, 1H), 4.15-3.98 (m, 3H), 3.03-2.74 (m, 6H), 1.43 (s, 6H), 1.39 (s, 9H), 1.35-0.91 (m, 6H).

### Example 200

### Preparation of 1-[4-({1-[(2R)-2-aminopropyl]-3,5-diethyl-1H-pyrazol-4-yl}sulfonyl)phenyl]-3-methyl-2,5-pyrrolidinedione

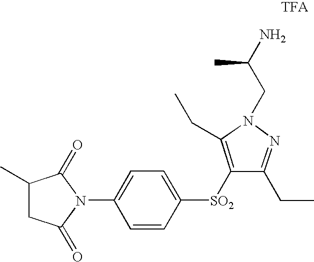

The compound was prepared using the same procedure described for Example 124 Product (0.17 g, 100%): MS (Electronspray) 448 (M+H)⁺, RT=2.88. Mp. 145-148° C. ¹H NMR (300 MHz, CDCl 3) δ 8.00 (d, 2H), 7.57 (d, 2H), 4.25 (dd, 2H), 3.81 (m, 1H), 3.03 (m, 2H), 2.83 (q, 2H), 2.78 (s, 2H), 1.39 (s, 6H), 1.32 (d, 3H, J=6.8 Hz), 1.26-1.16 (m, 6H). The compounds listed in the Tables 1-9 below were synthesized by the preparative methods described above or by using other known synthetic techniques in the art examples of which include those described by Schofield et al., Heteroaromatic Nitrogen Compounds: The Azoles, published by Cambridge University Press, (1976); and “Five Membered Heterocycles with Two Heteroatoms” from section 3 (1,2-Azoles), Chapter 4 of Heterocyclic Chemistry II—Five Membered Heterocycles, ed. by Gupta et al., publ. by Springer-Verlag, pages 435-454, (1999), each of which is incorporated in its entirety by reference.

Table 1 show examples 201-262 wherein:
n=0.

Table 2 show examples 263-290 wherein:
n=0,
R=4-fluorophenyl-.

Table 3 show examples 291-351 wherein:
n=0.

Table 4 show examples 352-361 wherein:
n=0,
R=R′-phenyl,
R₃=R₄=methyl.

Table 5 show examples 362-381 wherein:
n=0,
R=R′-phenyl.

Table 6 show examples 382-409 wherein:

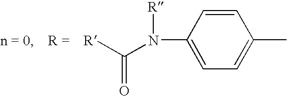

Table 7 show examples 410-425 wherein:

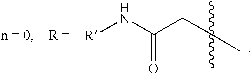

Table 8 show examples 426-429 wherein:

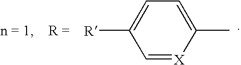

Table 9 show examples 430-512 wherein:
n 2,
R=R′-phenyl.

Table 10 shows analytical data accompanying the compounds of Table 9.

TABLE 1

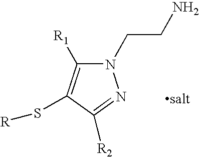

| | | | | HPLC RT | Mass Spec | | |
|---|---|---|---|---|---|---|---|
| Example | R | R₁ | R₂ | (min) | source | M.pt | salt |
| 201 | 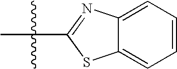 | Et | Et | | | | none |
| 202 | 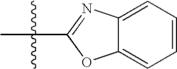 | Et | Et | | | | none |
| 203 | 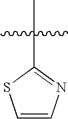 | Et | Et | | | | TFA |
| 204 | 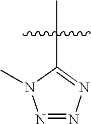 | Et | Et | 2.43 | 282 (M + H)+[electrospray] | 148 | maleic |
| 205 | 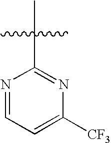 | Et | Et | 2.89 | 346 (M + H)+[electrospray] | 154 | maleic |
| 206 | 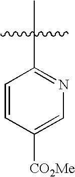 | Et | Et | | | | maleic |
| 207 | 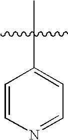 | Et | Et | | | | TFA |
| 208 | 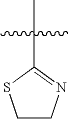 | Et | Et | | | | TFA |
| 209 | 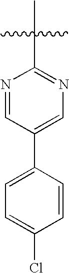 | Et | Et | | | | maleic |
| 210 | 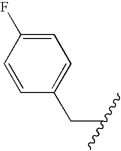 | Me | Me | | 232 (M + H)⁺[electrospray] | | TFA |
| 211 | 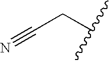 | Me | Me | 0.69* | 211 (M + H)⁺[electrospray] | | TFA |
| 212 | 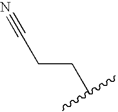 | Me | Me | 0.80* | 225 (M + H)⁺[electrospray] | | TFA |
| 213 | 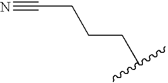 | Me | Me | 1.18* | 239 (M + H)⁺[electrospray] | | TFA |
| 214 | 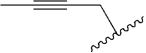 | Me | Me | 1.65* | 224 (M + H)⁺[electrospray] | | TFA |
| 215 | 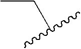 | Me | Me | 1.40* | 200 (M + H)⁺[electrospray] | | TFA |
| 216 | 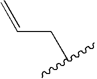 | Me | Me | 1.63* | 212 (M + H)⁺[electrospray] | | TFA |
| 217 | 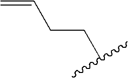 | Me | Me | 1.87* | 226 (M + H)⁺[electrospray] | | TFA |
| 218 | 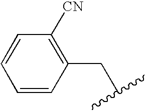 | Me | Me | 1.84* | 287 (M + H)⁺[electrospray] | | TFA |
| 219 | 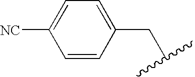 | Me | Me | 1.89* | 287 (M + H)⁺[electrospray] | | TFA |
| 220 | 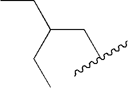 | Me | Me | 2.98 | 256 (M + H)⁺[electrospray] | | TFA |
| 221 | 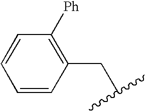 | Me | Me | 3.09 | 338 (M + H)⁺[electrospray] | | TFA |
| 222 | 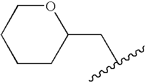 | Me | Me | 2.62 | 270 (M + H)⁺[electrospray] | | TFA |
| 223 | 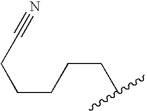 | Me | Me | 2.66 | 267 (M + H)⁺[electrospray] | | TFA |
| 224 | 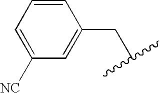 | Me | Me | 2.73 | 287 (M + H)⁺[electrospray] | | TFA |
| 225 | 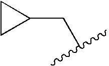 | Me | Me | 2.67 | 226 (M + H)⁺[electrospray] | | TFA |
| 226 | 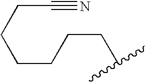 | Me | Me | 2.77 | 281 (M + H)⁺[electrospray] | | TFA |
| 227 | 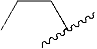 | Me | Me | 2.65 | 214 (M + H)⁺[electrospray] | | TFA |
| 228 | 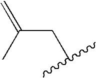 | Me | Me | 2.70 | 226 (M + H)⁺[electrospray] | | TFA |
| 229 | 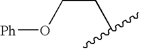 | Me | Me | 2.86 | 292 (M + H)⁺[electrospray] | | TFA |
| 230 | 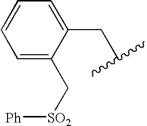 | Me | Me | 2.90 | 416 (M + H)⁺[electrospray] | | TFA |
| 231 | 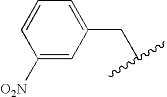 | Me | Me | 2.50 | 307 (M + H)⁺[electrospray] | | TFA |
| 232 | 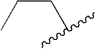 | Me | Me | 2.64 | 214 (M + H)⁺[electrospray] | | TFA |
| 233 | 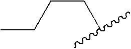 | Me | Me | 2.81 | 228 (M + H)⁺[electrospray] | | TFA |
| 234 | 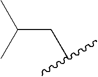 | Me | Me | 2.81 | 228 (M + H)⁺[electrospray] | | TFA |
| 235 | 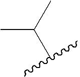 | Me | Me | 2.62 | 214 (M + H)⁺[electrospray] | | TFA |
| 236 | 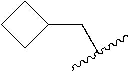 | Me | Me | 2.86 | 240 (M + H)⁺[electrospray] | | TFA |
| 237 | 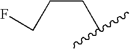 | Me | Me | 2.60 | 232 (M + H)⁺[electrospray] | | TFA |
| 238 | 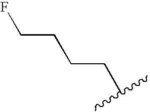 | Me | Me | 2.67 | 246 (M + H)⁺[electrospray] | | TFA |
| 239 | 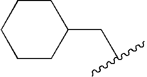 | Me | Me | 3.05 | 268 (M + H)⁺[electrospray] | | TFA |
| 240 | Me | Me | Me | 2.40 | 186 (M + H)⁺[electrospray] | | TFA |
| 241 | 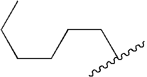 | Me | Me | 3.07 | 256 (M + H)⁺[electrospray] | | TFA |
| 242 | 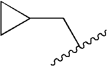 | Me | Me | 2.66 | 226 (M + H)⁺[electrospray] | | TFA |
| 243 | 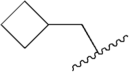 | Me | Me | 2.86 | 240 (M + H)⁺[electrospray] | | 2 HCl |
| 244 | 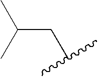 | Me | Me | 2.81 | 228 (M + H)⁺[electrospray] | | 2 HCl |
| 245 | 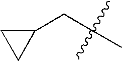 | Et | Et | 3.00 | 268 (M + H)⁺[electrospray] | | TFA |
| 246 | 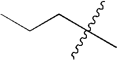 | Et | Et | 2.84 | 242 (M + H)⁺[electrospray] | | TFA |
| 247 | 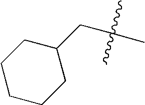 | Et | Et | 3.21 | 296 (M + H)⁺[electrospray] | | TFA |
| 248 | 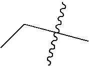 | Et | Et | 2.60 | 228 (M + H)⁺[electrospray] | | TFA |
| 249 | 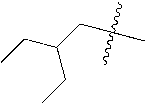 | Et | Et | 2.97 | 284 (M + H)⁺[electrospray] | | TFA |
| 250 | 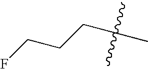 | Et | Et | 2.62 | 260 (M + H)⁺[electrospray] | | TFA |
| 251 | 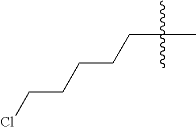 | Et | Et | 2.89 | 304 (M + H)⁺[electrospray] | | TFA |
| 252 | 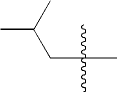 | Et | Et | 2.81 | 256 (M + H)⁺[electrospray] | | TFA |
| 253 | 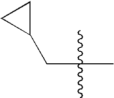 | Et | Et | 2.72 | 254 (M + H)⁺[electrospray] | | TFA |
| 254 | 5-NO₂—pyrid-2-yl- | Et | Et | | | 167 | TFA |
| 255 | 5-t-BuC(═O)NH—pyid-2-yl | Et | Et | | | | TFA |
| 256 | 5-cyc-PrCC(═O)NH—pyid-2-yl | Et | Et | | | | TFA |
| 257 | 5-cyc-HexC(═O)NH—pyid-2-yl | Et | Et | | | | TFA |
| 258 | 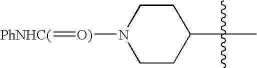 | Me | Me | 2.73 | 359 (M + H)⁺[electrospray] | | TFA |
| 259 | 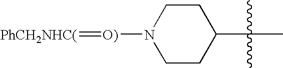 | Me | Me | 2.77 | 373 (M + H)⁺[electrospray] | | TFA |
| 260 | 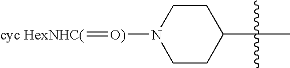 | Me | Me | 2.84 | 365 (M + H)⁺[electrospray] | | TFA |
| 261 | 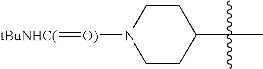 | Me | Me | 2.76 | 339 (M + H)⁺[electrospray] | | TFA |
| 262 | 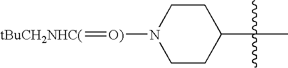 | Me | Me | 2.80 | 353 (M + H)⁺[electrospray] | | TFA |

TABLE 2

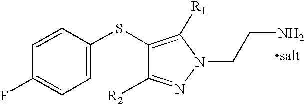

| | | | | Mass spec | |
|---|---|---|---|---|---|
| Example | R₁ | R₂ | HPLC RT (min) | [Source] | salt |
| 263 | 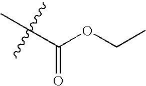 | Me | 3.30 | 324.1 | TFA |
| 264 | 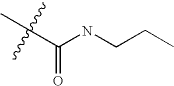 | Me | 3.16 | 337.1 | TFA |
| 265 | 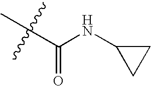 | Me | 3.06 | 335.1 | TFA |
| 266 | 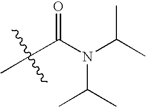 | Me | | | TFA |
| 267 | 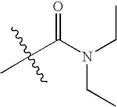 | Me | | | TFA |
| 268 | 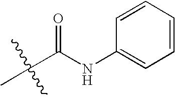 | Me | 2.32 | 371.2 | TFA |
| 269 | 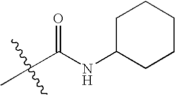 | Me | 2.42 | 377.3 | TFA |
| 270 | 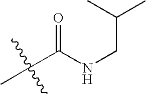 | Me | 3.63 | 351.1 | TFA |
| 271 | 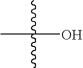 | Me | 2.77 | 268.1 | TFA |
| 272 | 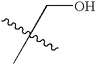 | Me | 2.71 | 282.0 | TFA |
| 273 | 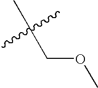 | Me | 2.81 | 296.0 | TFA |
| 274 | 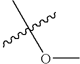 | Me | 2.74 | 282.1 | TFA |
| 275 | 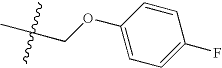 | Me | 3.08 | 376.0 | TFA |
| 276 | 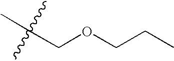 | Me | 2.92 | 324.0 | TFA |
| 277 | 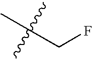 | Me | 2.80 | 284.0 | TFA |
| 278 | 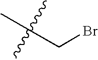 | Me | 2.98 | 345.9 | TFA |
| 279 | Me | 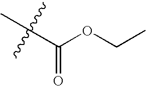 | | | TFA |
| 280 | Me | 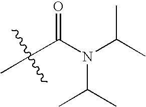 | 3.62 | 379.1 | TFA |
| 281 | Me | 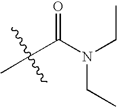 | 3.20 | 351.2 | TFA |
| 282 | Me | 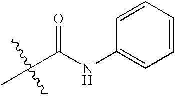 | 3.52 | 371.1 | TFA |
| 283 | Me | 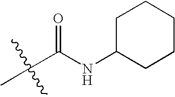 | 3.63 | 377.2 | TFA |
| 284 | Me | 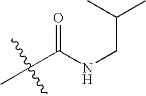 | 3.43 | 351.2 | TFA |
| 285 | Me | 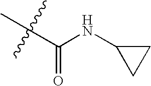 | 3.20 | 335.1 | TFA |
| 286 | Me | 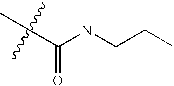 | 3.18 | 337.1 | TFA |
| 287 | Me | 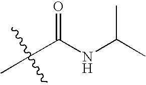 | 3.19 | 337.1 | TFA |
| 288 | Me | 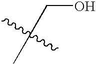 | 2.65 | 282.0 | TFA |
| 289 | Me | 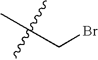 | 3.00 | 344.0/346.0 | TFA |
| 290 | Me | 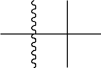 | 3.03 | 308.0 | TFA |

TABLE 3

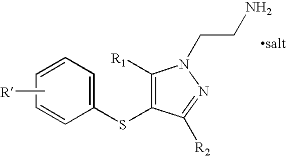

| | | | | | | Mass | | |
|---|---|---|---|---|---|---|---|---|
| | | | | | HPLC RT | Spec | | |
| Example | R₁ | | R₂ | (min) | [source] | M.pt | salt | |
| 291 | 4- | 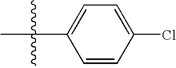 | Et | Et | 3.75 | 386 (M + H)+[electrospray] | | HCl |
| 292 | 4- | 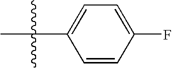 | Et | Et | 3.73 | 370 (M + H)+[electrospray] | | HCl |
| 293 | 4- | 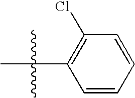 | Et | Et | 3.75 | 386 (M + H)+[electrospray] | | HCl |
| 294 | 4- | 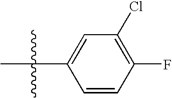 | Et | Et | 3.65 | 406 (M + H)+[electrospray] | | HCl |
| 295 | 4- | 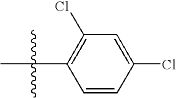 | Et | Et | 3.87 | 422 (M + H)+[electrospray] | | HCl |
| 296 | 4- | 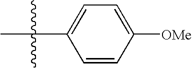 | Et | Et | 3.7 | (M + H)+[electrospray] | | |
| 297 | 4- | 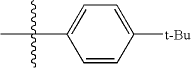 | Et | Et | 4.06 | 406 (M + H)+[electrospray] | | HCl |
| 298 | 4- | 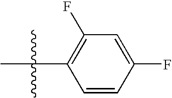 | Et | Et | 3.8 | 388 (M + H)+[electrospray] | | HCl |
| 299 | 4- | 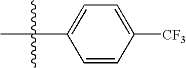 | Et | Et | 3.7 | 420 (M + H)+[electrospray] | 179 | maleic |
| 300 | 4- | 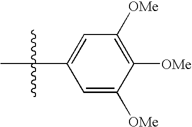 | Et | Et | 3.57 | 442 (M + H)+[electrospray] | 79 | maleic |
| 301 | 4- | 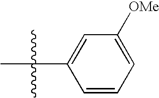 | Et | Et | 3.64 | 382 (M + H)+[electrospray] | 129 | maleic |
| 302 | 4- | 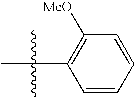 | Et | Et | 3.74 | 382 (M + H)+[electrospray] | 144 | maleic |
| 303 | 4- | 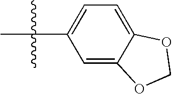 | Et | Et | 3.67 | 396 (M + H)+[electrospray] | 153 | maleic |
| 304 | 4- | 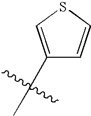 | Et | Et | 3.73 | 357 (M + H)+[electrospray] | 173 | maleic |
| 305 | 4- | 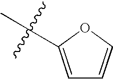 | Et | Et | 3.59 | 342 (M + H)+[electrospray] | | maleic |
| 306 | 4- | 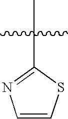 | Et | Et | 3.97 | 359 (M + H)+[electrospray] | | |
| 307 | 4- | 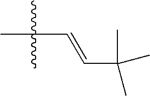 | Et | Et | 3.54 | (M + H)+[electrospray] | | |
| 308 | 4- | 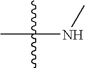 | Et | Et | | | | TFA |
| 309 | 4- | 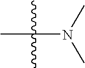 | Et | Et | | | | TFA |
| 310 | 4- | 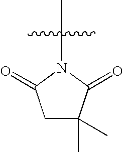 | Et | Et | | | 167 | HCl |
| 311 | 4- | 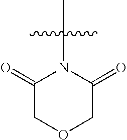 | Et | Et | | | 190 | HCl |

| | | | | | | | |
|---|---|---|---|---|---|---|---|
| 312 | H | Me | Me | 16.3 | 247 (M)⁺ | | |
| | | | | | [GC/MS]9 | | |
| 313 | H | Me | Me | | | 185 | HCl |
| 314 | 4-Cl | Me | Me | 18.99 | 281 | | |
| | | | | | (M)⁺ | | |
| | | | | | [GC/MS] | | |
| 315 | 4-OMe | Me | Me | 16.99 | 279 | | |
| | | | | | (M)⁺ | | |
| | | | | | [GC/MS] | | |
| 316 | 4-F | Me | Me | 15.85 | 265 | | |
| | | | | | (M)⁺ | | |
| | | | | | [GC/MS] | | |
| 307 | 3-OMe | Me | Me | 19.01 | 265 | | |
| | | | | | (M)⁺ | | |
| | | | | | [GC/MS] | | |
| 318 | 3-F | Me | Me | 2.07 | 266 | | HCl |
| | | | | | (M + H)+ | | |
| | | | | | [electrospray] | | |
| 319 | 4-CF3 | Me | Me | 2.32 | 316 | | HCl |
| | | | | | (M + H)+ | | |
| | | | | | [electrospray] | | |
| 320 | 3-CF3 | Me | Me | 2.31 | 316 | | HCl |
| | | | | | (M + H)+ | | |
| | | | | | [electrospray] | | |
| 321 | 4-NO2 | Me | Me | 3.13 | 293 | | TFA |
| | | | | | (M + H)+ | | |
| | | | | | [electrospray] | | |
| 322 | 4-NH2 | Me | Me | 2.47 | 263 | | TFA |
| | | | | | (M + H)+ | | |
| | | | | | [electrospray] | | |
| 323 | 4-F | Me | Me | 2.77 | 266 | | HCl |
| | | | | | (M + H)+ | | |
| | | | | | [electrospray] | | |
| 324 | 4-F | Et | Et | 2.31 | 299 | | HCl |
| | | | | | (M + H)+ | | |
| | | | | | [electrospray] | | |
| 325 | 4-OMe | Et | Et | | | | HCl |
| 326 | H | Et | Et | | | | HCl |
| 327 | 4-CF3 | Et | Et | | | | HCl |
| 328 | 3-F | Et | Et | | | | HCl |
| 329 | 3-CF3 | Et | Et | | | | HCl |
| 330 | 4-Br | Et | Et | 3.2 (FB) | 356 | | HCl |
| | | | | | (M + 2)+ | | |
| | | | | | [GC/MS] | | |
| 331 | 4-Cl | Et | Et | 2.41 | 310 | | HCl |
| | | | | | (M + H)+ | | |
| | | | | | [electrospray] | | |
| 332 | 4-Br | Et | Et | | | 169 | maleic |
| 333 | 3-OMe | Et | Et | | | | HCl |
| 334 | 3,4-OMe | Et | Et | | | 100 | maleic |
| 335 | 4-NO2 | Et | Et | 3.42 (FB) | 321 | 160 | maleic |
| | | | | | (M + H)+ | | |
| | | | | | [GC/MS] | | |
| 336 | 4-NH2 | Et | Et | 2.72 | 291 | | TFA |
| | | | | | (M + H)+ | | |
| | | | | | [electrospray] | | |
| 337 | 4-CO₂H | Et | Et | | | | TFA |
| 338 | 4-CO₂Me | Et | Et | | | | TFA |
| 339 | 4-F | Et | Et | | | 148 | maleic |
| 340 | 4-(4¹-F)PhSO₂NH— | Et | Et | | | | TFA |
| 341 | 4-MeSO₂NH— | Et | Et | | | | TFA |
| 342 | 4-MeSO₂NH— | Et | Et | | | 121 | Acetic |
| 343 | 4-iPrSO₂NH— | Et | Et | | | | TFA |
| 344 | 4-MeC(═S)NH— | Me | Me | 2.6 | 347 | | TFA |
| | | | | | (M + H)+ | | |
| | | | | | [electrospary] | | |
| 345 | 4-EtC(═S(NH) | Me | Me | 2.69 | 347 | | TFA |
| | | | | | (M + H)+ | | |
| | | | | | [electrospary] | | |
| 346 | 3-t-BuO(O)NH— | Et | Et | 2.29 | 375.4 | | TFA |
| 347 | 3-MeSO₂NH— | Et | Et | 2.85 | 369.0 | | TFA |
| 348 | 4-tBuNHO(═O)— | Et | Et | | | | TFA |
| 349 | 4-cyc-PrNHC(═O)— | Et | Et | | | | TFA |
| 350 | 4-F | Me | tBu | 3.00 | 308.1 | | TFA |
| 351 | 4-F | tBu | Me | 3.01 | 308.0 | | TFA |

TABLE 4

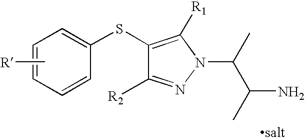

| | | | | HPLC RT | Mass Spec | | |
|---|---|---|---|---|---|---|---|
| Example | R′ | R₁ | R₂ | (min) | [source] | M.pt | salt |
| 352 | 3-CF3 | Me | Me | 4.15 | 344 | | TFA |
| | | | | | (M + H)+ | | |
| | | | | | [electrospary] | | |
| 353 | 3-F | Me | Me | 3.84 | 294 | | TFA |
| | | | | | (M + H)+ | | |
| | | | | | [electrospary] | | |
| 354 | 2-OMe | Me | Me | 3.84 | 306 | | TFA |
| | | | | | (M + H)+ | | |
| | | | | | [electrospary] | | |
| 355 | 4-Cl | Me | Me | 3.75 | 310 | | TFA |
| | | | | | (M + H)+ | | |
| | | | | | [electrospary] | | |
| 356 | 4-NO₂ | Et | Et | 4.07 | 349 | | TFA |
| | | | | | (M + H)+ | | |
| | | | | | [electrospary] | | |
| 357 | 4-OMe | Et | Et | 4.07 | 334 | | TFA |
| | | | | | (M + H)+ | | |
| | | | | | [electrospary] | | |
| 358 | 3-OMe | Et | Et | 4.01 | 334 | | TFA |
| | | | | | (M + H)+ | | |
| | | | | | [electrospary] | | |
| 359 | 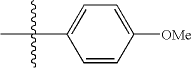 | Et | Et | 4.51 | 410 (M + H)+[electrospary] | | TFA |
| 360 | 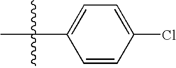 | Et | Et | 4.82 | 414 (M + H)+[electrospary] | | TFA |
| 361 | 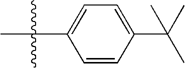 | Et | Et | 5.2 | 437 (M + H)+[electrospary] | | TFA |

TABLE 5

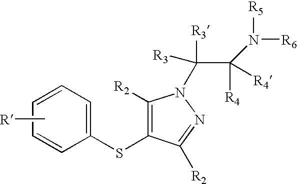

| Example | 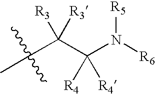 | R₁ | R₂ | R′ | HPLC RT (min) | Mass Spec [source] | M.pt | salt |
|---|---|---|---|---|---|---|---|---|
| 362 | 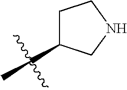 | Et | Et | F | 3.55 | 320 (M + H) +[electrospray] | | HCl |
| 363 | 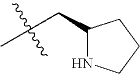 | Et | Et | F | 3.5 | 334 (M + H) +[electrospray] | | HCl |
| 364 | 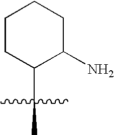 | Et | Et | F | 4.38 | 348 (M + H) +[electrospray] | | maleic |
| 365 | 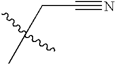 | Et | Et | F | 16.3 | 334 (M + H) +[GC/MS) | | |
| 366 | 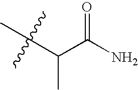 | Et | Et | F | 3.92 (FB) | 322 (M + ) +[electrospray) | | HCl |
| 367 | 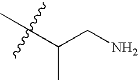 | Et | Et | F | 3.46 | 308 (M + H) +[electrospray) | | maleic |
| 368 | 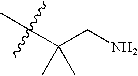 | Et | Et | F | 3.96 | 322 (M + H) +[electrospray] | | maleic |
| 369 | 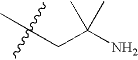 | Et | Et | F | | | 121 | maleic |
| 370 | 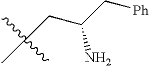 | Et | Et | F | 4.76 | 384 (M + H) +[electrospray] | | HCl |
| 371 | 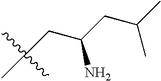 | Et | Et | F | 4.52 | 350 (M + H) +[electrospray] | | HCl |
| 372 | 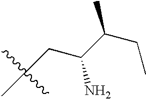 | Et | Et | F | 3.9 | 350 (M + H) +[electrospray] | | HCl |
| 373 | 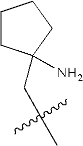 | Et | Et | F | 3.76 | 348 (M + H) +[electrospray] | | maleic |
| 374 | 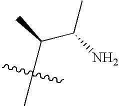 | Et | Et | F | 3.11 | 322 (M + H) +[electrospray] | | HCl |
| 375 | 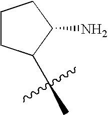 | Et | Et | F | 3.58 | 334 (M + H) +[electrospray] | 176 | maleic |
| 376 | 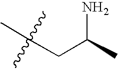 | Et | Et | F | 3.48 | 308 (M + H) +[electrospray] | | maleic |
| 377 | 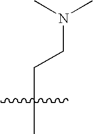 | Et | Et | F | 3.54 | 322 (M + H) +[electrospray] | | maleic |
| 378 | 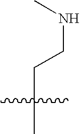 | Et | Et | F | 3.57 | 308 (M + H) +[electrospray] | 96 | maleic |
| 379 | 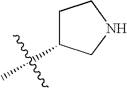 | Et | Et | F | 3.56 | 320 (M + H) +[electrospray] | | HCl |
| 380 | 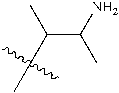 | Et | Et | F | 4.09 | 322 (M + H) +[electrospray] | | TFA |
| 381 | 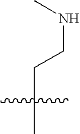 | Me | Me | t-BuC(═O)NH— | 2.05 | 3.61 (M + H) +[electrospray] | | TFA |

TABLE 6

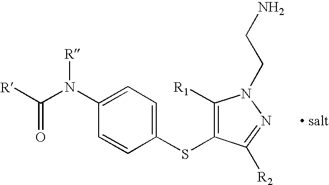

| | | | | | HPLC RT | Mass Spec | | |
|---|---|---|---|---|---|---|---|---|
| Example | R′ | R₁ | R₂ | R″ | (min) | [source] | M.pt | salt |
| 382 | 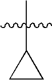 | Et | Et | H | | | 210 | TFA |
| 383 | 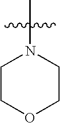 | Et | Et | H | | | 170 | TFA |
| 384 | CH₃ | Et | Et | H | | | 184 | TFA |
| 385 | 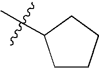 | Et | Et | H | | | 189 | TFA |
| 386 | 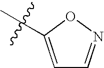 | Et | Et | H | | | 159 | TFA |
| 387 | 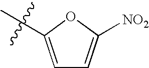 | Et | Et | H | | | 227 | TFA |
| 388 | 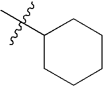 | Et | Et | H | | | 174 | TFA |
| 389 | 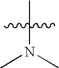 | Et | Et | H | | | | TFA |
| 390 | 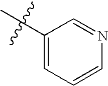 | Et | Et | H | | | 91 | TFA |
| 391 | 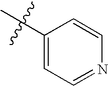 | Et | Et | H | | | 72 | TFA |
| 392 | 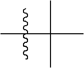 | Et | Et | H | | | 191 | TFA |
| 393 | 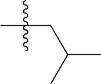 | Et | Et | H | | | 196 | TFA |
| 394 | 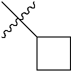 | Et | Et | H | | | 141 | TFA |
| 395 | 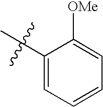 | Et | Et | H | | | 61 | TFA |
| 396 | 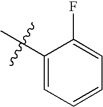 | Et | Et | H | | | 141 | TFA |
| 397 | 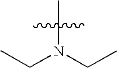 | Et | Et | H | | | | TFA |
| 398 | 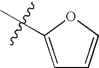 | Et | Et | H | | | | TFA |
| 399 | 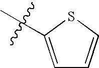 | Et | Et | H | | | | TFA |
| 400 | 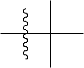 | Et | Et | Me | | | | TFA |
| 401 | 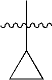 | Et | Et | Me | | | | TFA |
| 402 | t-Bu | Et | Et | H | 2.3 | 361 (M+H)+[electrospray] | | TFA |
| 403 | 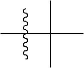 | Me | Me | H | 3.2 | 347 (M+H)+[electrospry] | | TFA |
| 404 | 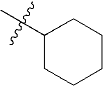 | Me | Me | H | 2.93 | 373 (M+H)+[electrospry] | | TFA |
| 405 | 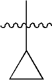 | Me | Me | H | 2.7 | 331 (M+H)+[electrospry] | | TFA |
| 406 | 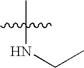 | Me | Me | H | 2.62 | 334 (M+H)+[electrospry] | | TFA |
| 407 | 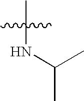 | Me | Me | H | 2.69 | 348 (M+H)+[electrospry] | | TFA |
| 408 | 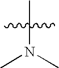 | Me | Me | H | 2.62 | 334 (M+H)+[electrospry] | | TFA |
| 409 | 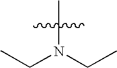 | Me | Me | H | 2.75 | 362 (M+H)+[electrospry] | | TFA |

TABLE 7

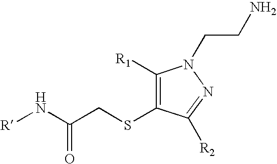

| | | | | | Mass | | |
|---|---|---|---|---|---|---|---|
| | | | | HPLC RT | Spec | | |
| Example | R′ | R₁ | R₂ | (min) | [source] | M.pt | salt |
| 410 | 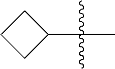 | Me | Me | 2.29 | 283 | | TFA |
| 411 | 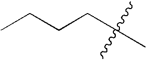 | Me | Me | 2.23 | 2.71 | | TFA |
| 412 | 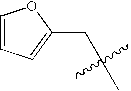 | Me | Me | 2.38 | 309 | | TFA |
| 413 | 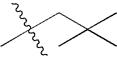 | Me | Me | 2.62 | 299 | | TFA |
| 414 | 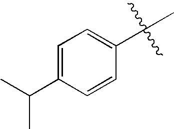 | Me | Me | 2.93 | 347 | | TFA |
| 415 | 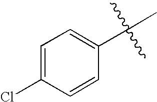 | Me | Me | 2.81 | 339 | | TFA |
| 416 | 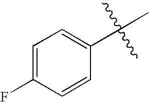 | Me | Me | 2.64 | 323 | | TFA |
| 417 | 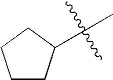 | Me | Me | 2.5 | 297 | | TFA |
| 418 | 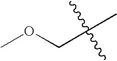 | Me | Me | 2.14 | 287 | | TFA |
| 419 | 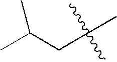 | Me | Me | 2.42 | 285 | | TFA |
| 420 | 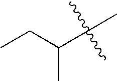 | Me | Me | 2.43 | 285 | | TFA |
| 421 | 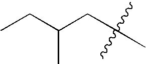 | Me | Me | 2.58 | 299 | | TFA |
| 422 | 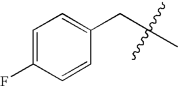 | Me | Me | 2.6 | 337 | | TFA |
| 423 | 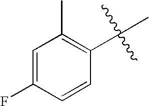 | Me | Me | 2.61 | 337 | | TFA |
| 424 | 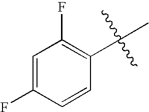 | Me | Me | 2.58 | 341 | | TFA |
| 425 | 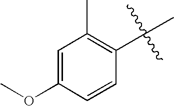 | Me | Me | 2.6 | 349 | | TFA |

TABLE 8

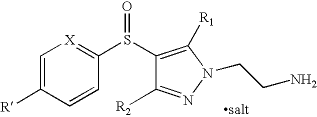

| | | | | | HPLC | | | |
|---|---|---|---|---|---|---|---|---|
| | | | | | RT | Mass Spec | | |
| Example | R′ | R₁ | R₂ | X | (min) | [source] | M.pt | salt |
| 426 | t-BuC(═O)NH— | Et | Et | N | | | | TFA |
| 427 | [cyc-PrC(═O)]₂NH— | Et | Et | N | 3.4 | 442 (M + H)+ | | TFA |
| | | | | | | [electrospray] | | |
| 428 | Cl | Et | Et | CH | | | 160 | maleic |
| 429 | F | Et | Et | CH | 1.73 | 310 (M + H)+ | | TFA |
| | | | | | | [electrospray] | | |

TABLE 9

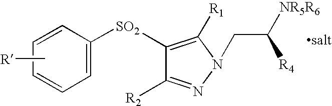

| Ex- | | | | | | | |
|---|---|---|---|---|---|---|---|
| am- | | | | | | | |
| ple | R′ | | R₁ | R₂ | R₅ | R₆ | R₄ |
| 430 | 4-F | | Me | Me | H | H | H |
| 431 | 4- | 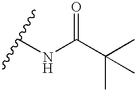 | Me | Me | H | H | H |
| 432 | 4- | 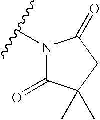 | Me | Me | H | H | H |
| 433 | 4- | 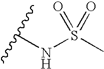 | Me | Me | H | H | H |
| 434 | 4- | 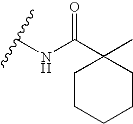 | Me | Me | H | H | H |
| 435 | 4- | 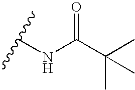 | Me | Me | H | Me | H |
| 436 | 4- | 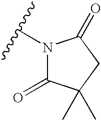 | Me | Me | H | Me | H |
| 437 | 4- | —NHC(═O)t-Bu | Me | Me | Me | Me | H |
| 438 | 4- | —NHSO₂Me | Me | Me | Me | Me | H |
| 439 | 4- | 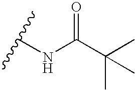 | Me | Me | H | H | Me |
| 440 | 4- | 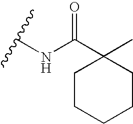 | Me | Me | H | H | Me |
| 441 | 4- | 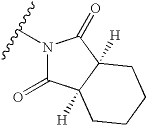 | Me | Me | H | H | Me |
| 442 | 4- | 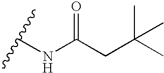 | Me | Me | H | H | Me |
| 443 | 4- | 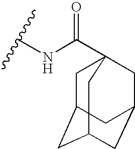 | Me | Me | H | H | Me |
| 444 | 4- | 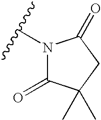 | Me | Me | H | H | Me |
| 445 | 4- | 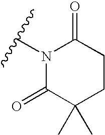 | Me | Me | H | H | Me |
| 446 | 4- | 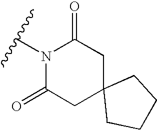 | Me | Me | H | H | Me |
| 447 | 4- | 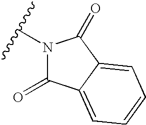 | Me | Me | H | H | Me |
| 448 | 4-Br | | Et | Et | H | H | H |
| 449 | 4-Cl | | Et | Et | H | H | H |
| 450 | 4-F | | Et | Et | H | H | H |
| 451 | 4-OMe | | Et | Et | H | H | H |
| 452 | 4- | 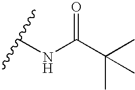 | Et | Et | H | H | H |
| 453 | 4- | 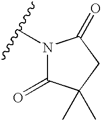 | Et | Et | H | H | H |
| 454 | 4- | 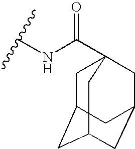 | Et | Et | H | H | H |
| 455 | 4- | 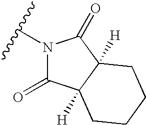 | Et | Et | H | H | H |
| 456 | 4- | 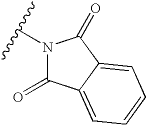 | Et | Et | H | H | H |
| 457 | 4-F | | Et | Et | Me | H | H |
| 458 | 4- | 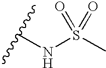 | Et | Et | Me | H | H |
| 459 | 4- | 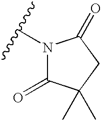 | Et | Et | Me | H | H |
| 460 | 4- | 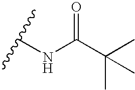 | Et | Et | Me | H | H |
| 461 | 4-F | | Et | Et | H | H | Me |
| 462 | 4- | 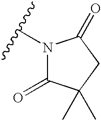 | Et | Et | H | H | Me |
| 463 | 4- | 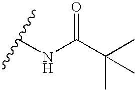 | Et | Et | H | H | Me |
| 464 | 4- | 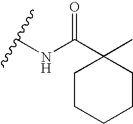 | Et | Et | H | H | Me |
| 465 | 4- | 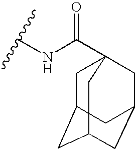 | Et | Et | H | H | Me |
| 466 | 4- | 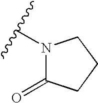 | Et | Et | H | H | Me |
| 467 | 4- | 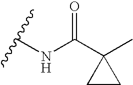 | Et | Et | H | H | Me |
| 468 | 4- | 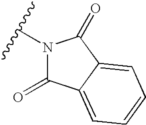 | Et | Et | H | H | Me |
| 469 | 4- | 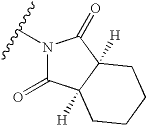 | Et | Et | H | H | Me |
| 470 | 4- | 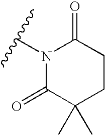 | Et | Et | H | H | Me |
| 471 | 4- | 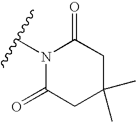 | Et | Et | H | H | Me |
| 472 | 4- | 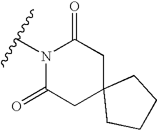 | Et | Et | H | H | Me |
| 473 | 4- | 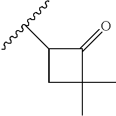 | Et | Et | H | H | Me |
| 474 | 4- | 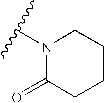 | Et | Et | H | H | Me |
| 475 | 4- | 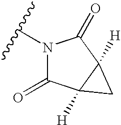 | Et | Et | H | H | Me |
| 476 | 4- | 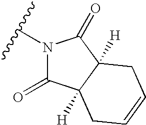 | Et | Et | H | H | Me |
| 477 | 4- | 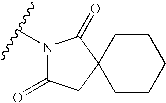 | Et | Et | H | H | Me |
| 478 | 4- | 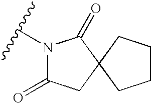 | Et | Et | H | H | Me |
| 479 | 4- | 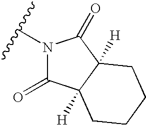 | Et | Et | H | H | Me |
| 480 | 4- | 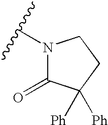 | Et | Et | H | H | Me |
| 481 | 4- | 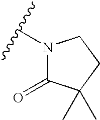 | Et | Et | H | H | Me |
| 482 | 4- | 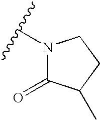 | Et | Et | H | H | Me |
| 483 | 4- | 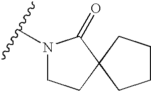 | Et | Et | H | H | Me |
| 484 | 4- | 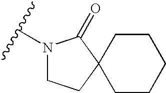 | Et | Et | H | H | Me |
| 485 | 4- | 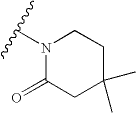 | Et | Et | H | H | Me |
| 486 | 4- | 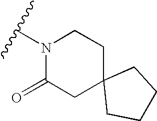 | Et | Et | H | H | Me |
| 487 | 4- | 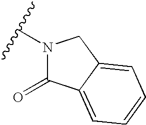 | Et | Et | H | H | Me |
| 488 | 4- | 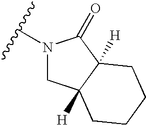 | Et | Et | H | H | Me |
| 489 | 4- | 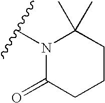 | Et | Et | H | H | Me |
| 490 | 4- | 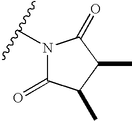 | Et | Et | H | H | Me |
| 491 | 4- | 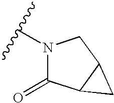 | Et | Et | H | H | Me |
| 492 | 4- | 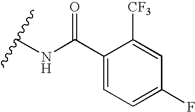 | Et | Et | H | H | Me |
| 493 | 4- | 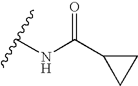 | Et | Et | H | H | Me |
| 494 | 4- | 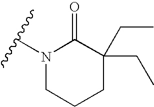 | Et | Et | H | H | Me |
| 495 | 4- | 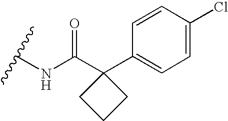 | Et | Et | H | H | Me |
| 496 | 4- | 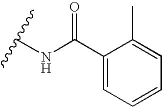 | Et | Et | H | H | Me |
| 497 | 4- | 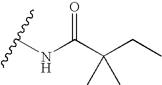 | Et | Et | H | H | Me |
| 498 | 4- | 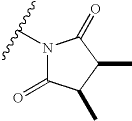 | Et | Et | H | H | Me |
| 499 | 4- | 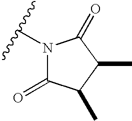 | Et | Et | H | H | Me |
| 500 | 3- | 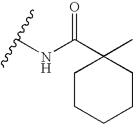 | Et | Et | H | H | Me |
| 501 | 3- | 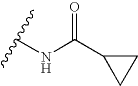 | Et | Et | H | H | Me |
| 502 | 3- | 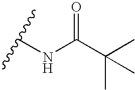 | Et | Et | H | H | Me |
| 503 | 3- | 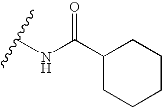 | Et | Et | H | H | Me |
| 504 | 3- | 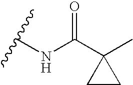 | Et | Et | H | H | Me |
| 505 | 3- | 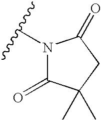 | Et | Et | H | H | Me |
| 506 | 3- | 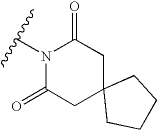 | Et | Et | H | H | Me |
| 507 | 3- | 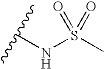 | Et | Et | H | H | Me |
| 508 | 3-Cl-4-NHC(═O)t-Bu | | Et | Et | H | H | Me |
| 509 | 3-Br-4-NHC(═O)t-Bu | | Et | Et | H | H | Me |
| 510 | 4-F | | Et | Et | Me | H | Me |
| 511 | 4- | 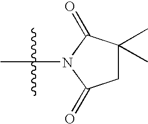 | Et | Et | Me | Me | H |
| 512 | 4- | 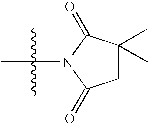 | Et | Et | H | H | 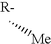 |

TABLE 10

| | HPLC | | | |
|---|---|---|---|---|
| | RT | | | |
| Example | (min) | Mass Spec | M.pt | Salt |
| 430 | | | | MA |
| 431 | | | | TFA |
| 432 | 2.67 | 405 (M + H)⁺ | 82-85 | TFA |
| 433 | | | 53 | TFA |
| 434 | 2.23 | 419 | | TFA |
| 435 | | | | TFA |
| 436 | | | 71 | MA |
| 437 | | | | |
| 438 | | | | |
| 439 | | | | TFA |
| 440 | | | | TFA |
| 441 | | | | |
| 442 | 2.81 | 407 | | TFA |
| 443 | 3.10 | 471 | | TFA |
| 444 | | | | TFA |
| 445 | 2.58 | 433 | | TFA |
| 446 | 2.78 | 459 | | TFA |
| 447 | 2.81 | 439 | | TFA |
| 448 | | | | HCl |
| 449 | | | | HCl |
| 450 | 1.95 | 326 | | HCl |
| 451 | | | | TFA |
| 452 | | | | TFA |
| 453 | 2.80 | 433 (M + H)⁺ | | TFA |
| 454 | | | | TFA |
| 455 | 2.83 | 459 (M + H)⁺ | | TFA |
| 456 | 2.91 | 453 (M + H)⁺ | | TFA |
| 457 | | | | MA |
| 458 | | | | TFA |
| 459 | 2.87 | | 84-86 | TFA |
| 460 | | | | TFA |
| 461 | | | | TFA |
| 462 | 2.88 | 447 (M + H)⁺ | | |
| 463 | | | | TFA |
| 464 | | | | TFA |
| 465 | | | | TFA |
| 466 | | | | TFA |
| 467 | | | | TFA |
| 468 | 3.08 | 467 (M + H)⁺ | | TFA |
| 469 | 3.01 | 473 (M + H)⁺ | | TFA |
| 470 | 2.89 | 461 (M + H)⁺ | | TFA |
| 471 | 2.86 | 461 (M + H)⁺ | | TFA |
| 472 | 3.02 | 487 (M + H)⁺ | | TFA |
| 473 | | | | HCl |
| 474 | 1.88 | 419 | | HCl |
| 475 | 1.89 | 431 (M + H)⁺ | | HCl |
| 476 | 2.91 | 471 (M + H)⁺ | | HCl |
| 477 | 3.01 | 487 | | HCl |
| 478 | 2.91 | 473 | | HCl |
| 479 | 2.15 | 473 | | TFA |
| 480 | 2.69 | 557 | | HCl |
| 481 | 2.15 | 433 (M + H)⁺ | | HCl |
| 482 | 2.03 | 419 | | HCl |
| 483 | | | | HCl |
| 484 | | | | HCl |
| 485 | | | | HCl |
| 486 | 2.26 | 473 | | HCl |
| 487 | 2.23 | 453 (M + H)⁺ | | HCl |
| 488 | 2.36 | 459 (M + H)⁺ | | HCl |
| 489 | 2.12 | 447 (M + H)⁺ | | HCl |
| 490 | 2.13 | 447 | | HCl |
| 491 | 1.94 | 417 | | HCl |
| 492 | 2.33 | 527 | | HCl |
| 493 | 2.06 | 405 (M + H)⁺ | | HCl |
| 494 | 2.41 | 475 (M + H)⁺ | | HCl |
| 495 | 2.66 | 529 | | HCl |
| 496 | 2.24 | 455 | | HCl |
| 497 | 2.24 | 435 (M + H)⁺ | | HCl |
| 498 | 2.04 | 447 | | SA |
| 499 | | | | Mesylate |
| 500 | 2.23 | 461 | | HCl |
| 501 | 1.94 | 405 | | HCl |
| 502 | 2.10 | 421 | | HCl |
| 503 | 2.23 | 447 | | HCl |
| 504 | 1.98 | 419 | | HCl |
| 505 | 1.98 | 447 | | HCl |
| 506 | 2.17 | 487 | | HCl |
| 507 | 1.74 | 415 | | HCl |
| 508 | 2.38 | 455 | | TFA |
| 509 | 2.42 | 499 | | HCl |
| 510 | | | | TFA |
| 511 | 2.85 | 461 (M + H)+ | | |
| 512 | 2.71 | 447 | | TFA |

### Description of Method of Use

The compounds of formulas (I) and (II) interact with the 5-HT_{2C} receptor and are used in the treatment or prevention of diseases and/or behaviors that involve the 5-HT_{2C} receptor. These diseases and/or behaviors include obesity, obesity related disorders such as diabetes, feeding behavior, eating disorders such as bulimia, anorexia nervosa and premenstrual tension.

Further diseases and/or behaviors which can be treated or prevented include central nervous disorders, depressions, anxiety disorders, obsessive-compulsive disorders, sleep disorders, sexual dysfunction, psychoses, migraine, schizophrenia, drug or alcohol addiction and chronic fatigue syndrome.

Obesity is considered a major medical problem largely because it is a factor for a number of other diseases, and obese individuals have a higher chance of dying at a younger age than their leaner counterparts. Obesity is correlated with a much higher incidence of Type II diabetes (NIDDM), hypertension, hyperlipidemia, myocardial infarction, cancers, gallbladder disease, respiratory disease, gout, arthritis, and dermatological disease.

Targeting the 5-HT_{2C} receptor as method of treating obesity has previously been described (J. Pharmacology, 141, 429-435, (1987) and Psychopharmacology, 96, 93-100, (1988) each of which is hereby incorporated by reference). Agonists that are selective for this receptor would be expected to have superior properties with respect to other known appetite suppressants, such as serotonin/noradrenaline re-uptake inhibitors, which can lead to hypertension and/or cardiac valve defects.

Serotonin has been implicated in the regulation of feeding behavior and the infusion of 5-HT into the brain, resulting in lower food intake by promoting satiety. Furthermore, drugs which increase the concentration of 5-HT in the synaptic cleft by increasing 5-HT release and/or inhibiting re-uptake of the transmitter (such as Redux® (dexfenfluramine) and sibutramine) are effective long term treatments for obesity. However, while activation of several (5-HT_{1A}, 5-HT_{1B}, 5-HT_{2A}, and 5-HT_{2C}) subtypes of 5-HT receptors has been demonstrated to elicit effects on food intake, the best data available to date suggests that 5-HT_{2C} receptor agonists produce a decrease in food intake which is associated with the least likely potential for side effects. 5-HT_{2C} receptors are localized to the hypothalamus and the brainstem, two brain regions known to play a critical role in the modulation of food intake.

Serotonin produces physiological effects by acting on a heterogeneous family of receptors. The lack of selective agonists and antagonists for all of the individual subtypes of serotonin receptors has prevented a complete characterization of the physiological role of each receptor subtype.

Activation of both 5-HT_{2A} and 5-HT_{2C} receptors decrease food intake. However, while the 5-HT_{2C} receptor has been implicated in the regulation of satiety, 5-HT_{2A} receptor agonists are thought to decrease food intake by disrupting the ability of the animal to feed. Non-selective agonists/partial agonists (mCPP, TFMPP) at the 5-HT_{2C} receptor have been shown to reduce food intake in rats and to accelerate the appearance of the behavioral satiety sequence. Importantly, the hypophagic effects of mCPP are antagonized by the highly selective (at least 100-fold selective) 5-HT_{2C} receptor antagonist SB-242084. Recent findings from studies in normal human volunteers and obese subjects administered mCPP have also shown decreases in food intake. Thus, a single injection of mCPP decreased food intake in female volunteers and subchronic treatment for a 14 day period decreased the appetite and body weight of obese male and female subjects.

Although mCPP is a non-selective 5-HT agonist, the observations that the anorectic action of the drug is:
(a) absent in 5-HT_{2C} knockout mice; and
(b) antagonized by the 5-HT_{2C} receptor antagonist SB-242084 in rats,
   suggests that it decreases food intake via an agonist action at the 5-HT_{2C} receptor. Therefore, both animal and human data strongly implicate the involvement of the 5-HT_{2C} receptor in satiety.

Antagonist studies have shown that the selective 5-HT_{2C} receptor antagonist SB-242084 is highly effective in reversing the hypophagic actions of dexfenfluramine in the rat. Furthermore, the 5-HT₂ receptor antagonist, ritanserin, reversed the anorectic effect of dexfenfluramine in human volunteers. As ritanserin has a 10,000-fold selectivity for the 5-HT₂ receptors (pKi 8.9) over 5-HT, receptors, a crucial role for the 5-HT₂ receptors in the anorectic action of dexfenfluramine in humans is suggested.

The importance of the 5-HT_{2C} receptor in mediating feeding behavior is further supported by studies on mutant 5-HT_{2C}-knockout mice lacking this receptor (Nature, 374, 542-546 9(1995) and British Journal of Pharmacology, 128, 113-209 (1999), which is hereby incorporated by reference). Interestingly, the knockout mice show significantly greater weight gain and adipose tissue deposits over time compared to wild-type mice. Additional studies have confirmed that 5-HT_{2C} knockout mice overeat and become obese which appears due to a defect in their satiety mechanism. In the behavioral satiety sequence model, knockout animals continued to eat for a significantly longer period of time than the wild-type controls. The prolonged eating in the 5-HT_{2C} receptor knockout mice was enhanced by access to a sweet diet, suggesting that the 5-HT_{2C} receptor may play a role in palatability.

It is significant that the decrease in food intake induced by dexfenfluramine is markedly attenuated in 5-HT_{2C} receptor knockout mice. These results suggest that dexfenfluramine enhances satiety and decreases food intake via an agonist action on 5-HT_{2C} receptors. In addition, in wild-type animals these anorectic effects of dexfenfluramine are blocked by the 5-HT_{2C}-selective antagonist SB-242084. These data are consistent with the clinical evidence that the anorectic effect of dexfenfluramine was blocked by the 5HT₂ receptor antagonist ritanserin.

Thus, anorectic activity of the compounds of formulas (I) and (II) can be determined by measurement of their binding affinity to the 5-HT_{2C} receptor. Other research groups have explored this approach and have disclosed a number of ligands for the S-HT_{2C} receptor. (Cerebrus Pharmaceuticals: WO 00/12502, WO 00/12481, WO 00/12475, WO 00/12510, WO 00/12482; Hoffman-La Roche: U.S. Pat. No. 5,292,732, U.S. Pat. No. 5,646,173; Yamanouchi.

Pharmaceutical: WO98/56768; and Akzo Nobel: EP 0 863 136 A1, each of which is hereby incorporated by reference).

The following assay was performed to determine the effect of the compounds of formulas (1) and (II) on the 5-HT_{2C} receptor:

AV-12 cell pellets expressing 5-HT_{2C}, 5-HT_{2A} or 5-HT_{2B} receptors are homogenized in binding buffer (50 mM Tris-HCl, 10 mM MgCl₂, 10 uM pargyline, 0.1% Sodium Ascorbate, 0.5 mM EDTA, pH 7.4 using saturated Tris Base). Radioligand binding assays were performed as follows: 50 μL of various concentrations of test compound or reference compound (5-HT) are added to 50 μL of ¹²⁵I-DOI (1-(2,5-dimethoxy-4-iodophenyl)-2-aminopropane). Non-specific binding is defined by 10 uM 5-HT. The reaction is initiated by the addition of 100 μL membrane homogenate and incubated for 45 minutes at room temperature (23° C.). Bound radioactivity is determined after rapid filtration using a Brandel Cell Harvester. Filter plates (GF/B pretreated with 0.5% polyethyleneimine) are washed twice with ice-cold wash buffer (50 mM Tris-HCl, pH 7.4 using saturated Tris Base) and radioactivity determined using a Microbeta counter. Data (IC₅₀ values) are analyzed using a four parameter logistic equation (Graph Pad).

All example compounds of formulas I and II were tested in the above assays and were found to have an effect on 5-HT_{2C} at or below a concentration of 10 μM.

Other embodiments of the invention will be apparent to the skilled in the art from a consideration of this specification or practice of the invention disclosed herein. It is intended that the specification and examples be considered as exemplary only, with the scope and spirit of the invention being indicated by the following claims.

1. A compound of formula: 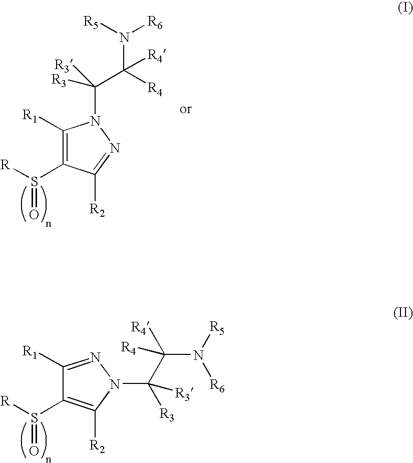
wherein:
n is 0, 1 or 2;
R is selected from the group consisting of:
(a) (C₁-C₆)-alkyl optionally substituted by a substituent selected from the group consisting of:
(b1) halogen,
(b2) cyano,
(b3) (C₁-C₅)-alkoxy,
(b4) (C₆-C₁₀)-aryloxy,
(b5) C(═O)NR₇R₈,
(b6) (C₃-C₈)-cycloalkyl, and
(b7) (C₆-C₁₀)-aryl optionally substituted with one to three substituents selected from the group consisting of cyano, halogen, nitro, (C₁-C₅)-alkyl, (C₁-C₅)-alkoxy, phenyl and arylsulfonyl,
(b) (C₁-C₅)-alkenyl optionally substituted with (C₁-C₅)-alkyl,
(c) (C₁-C₅)-alkynyl optionally substituted with (C₁-C₅)-alkyl,
(d) (C₆-C₁₀)-aryl which is optionally substituted with one to three substituents selected from the group consisting of:
(d1) halogen,
(d2) nitro,
(d3) (C₁-C₅)-alkyl optionally substituted with halogen,
(d4) (C₁-C₅)-alkenyl optionally substituted with (C₁-C₅)-alkyl,
(d5) (C₁-C₅)-alkynyl optionally substituted with (C₁-C₅)-alkyl,
(d6) (C₁-C₅)-alkoxy,
(d7) NR₉C(═O)R₁₀,
(d8) NR₉S(═O)ₙ—R₁₀,
(d9) NR₉C(═S)R₁₀,
(d10) NR₁₁R₁₂,
(d11) C(═O)R₁₀,
(d12) C(═O)NR₁₃R₁₄,
(d13) C(═O)OR₁₅,
(d14) (C₆-C₁₀)-aryl optionally substituted with one to three substituents selected from the group consisting of:
(d14a) halogen,
(d14b) (C₁-C₅)-alkyl,
(d14c) (C₁-C₅)-alkoxy,
(d14d) a four to eight membered saturated or unsaturated heterocyclic ring which contains one to four heteroatoms selected from the group consisting of nitrogen, oxygen and sulfur, wherein said heterocyclic ring contains at least one carbon atom and wherein said heterocyclic ring is optionally substituted with one to four substituents selected from the group consisting of:
(d14d1) nitro,
(d14d2) NR₉C(═O)R₁₀,
(d14d3) oxo,
(d14d4) (C₁-C₅) alkyl optionally substituted with halogen,
(d14d5) C(═O)R₁₅,
(d14d6) C(═O)OR₁₅,
(d14d7) C(═O)NR₁₃R₁₄,
(d14d8) (C₆-C₁₀)-aryl optionally substituted with halogen, and
(d14d9) (C₃-C₈)-cycloalkyl ring, and
(d14e) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atom and said heterocyclic ring is optionally substituted with one to two oxo substituents, and the other ring is a saturated or unsaturated three to eight membered cycloalkyl ring,
(d15) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atom, and the other ring is a saturated or unsaturated three to eight membered cycloalkyl ring;
(d16) C(═O)OR₁₅,
(d17) OH, and
(d18) CN;
(e) a four to eight membered saturated or unsaturated heterocyclic ring which contains one to four heteroatoms selected from the group consisting of nitrogen, oxygen and sulfur wherein said heterocyclic ring contains at least one carbon atom wherein said heterocyclic ring is optionally substituted with one to four substituents selected from the group consisting of.
(e1) nitro,
(e2) NR₉C(═O)R₁₀,
(e3) oxo,
(e4) (C₁-C₅)-alkyl optionally substituted with halogen,
(e5) C(═O)R₁₅,
(e6) C(═O)OR₁₅,
(e7) C(═O)NR₁₃R₁₄,
(e8) (C₆-C₁₀)-aryl optionally substituted with halogen, and
(e9) (C₃-C₈)-cycloalkyl ring, and
(f) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atoms, and the other ring is a saturated or unsaturated three to eight membered cycloalkyl ring;
R₁ and R₂ are independently selected from the group consisting of:
(a) hydrogen,
(b) hydroxy,
(c) (C₁-C₅)-alkyl optionally substituted with halogen or hydroxy,
(d) (C₁-C₅)-alkoxy,
(e) (C₁-C₅)-alkoxy-(C₁-C₅)-alkyl,
(f) (C₆-C₁₀)-aryl-(C₁-C₅)-alkoxy- wherein the (C₆-C₁₀)-aryl is optionally substituted with halogen,
(g) C(═O)R₁₅, and
(h) C(═O)NR₁₇R₁₈;
R₃, R_{3′}, R₄ and R₄ are independently selected from the group consisting of
(a) hydrogen,
(b) (C₁-C₅)-alkyl,
(c) (c₆-C₁₀)-aryl,
(d) (C₆-C₁₀)-aryl-(C₁-C₅)-alkyl, and
(e) (C₃-C₈)-cycloalkyl ring,
R₃ and R₄ together form a four to eight membered saturated or unsaturated carbocyclic ring, or
R₄ and R₄ together form a (C₃-C₈)-cycloalkyl ring;
R₅ and R₆ are independently selected from the group consisting of hydrogen and (C₁-C₅)-alkyl, or
the carbon to which R₄ and R₄ are attached and NR₅R₆ form a —CN wherein R₄ and R₅ form a bond and R₄ and R₆ form a bond, or
R₃, R₄ and NR₅R₆ together form a four to eight membered saturated or unsaturated heterocyclic ring wherein the nitrogen represents the only heteroatom;
R₇ and R₈ are independently selected from the group consisting of:
(a) hydrogen,
(b) (C₁-C₆)-alkyl optionally substituted with (C₁-C₅)-alkoxy or a four to eight membered heterocyclic ring which contains one to four heteroatoms selected from the group consisting of nitrogen, oxygen and sulfur wherein said heterocyclic ring contains at least one carbon atom,
(c) (C₆-C₁₀)-aryl optionally substituted with one to three substituents selected from the group consisting of halogen, (C₁-C₅)-alkoxy or (C₁-C₅)-alkyl optionally substituted by halogen,
(d) (C₆-C₁₀)-aryl-(C₁-C₅)-alkyl wherein the (C₆-C₁₀)-aryl is optionally substituted with one to three substituents selected from the group consisting of halogen, (C₁-C₅)-alkoxy or (C₁-C₅)-alkyl optionally substituted by halogen, and
(e) (C₃-C₈)-cycloalkyl is optionally substituted with one to three substituents selected from the group consisting of halogen, (C₁-C₅)-alkyl, or (C₁-C₅)— alkoxy;
R₉ is hydrogen or (C₁-C₅)-alkyl;
R₁₀ is selected from the group consisting of:
(a) (C₁-C₅)-alkyl optionally substituted with C₃-C₈-carbocyclic ring or C₆-C₁₀-aryl optionally substituted with halogen,
(b) (C₁-C₅)-alkoxy,
(c) (C₃-C₈)-cycloalkyl optionally substituted with one to three substituents selected from the group consisting of halogen, (C₁-C₅)-alkoxy and (C₁-C₅)-alkyl optionally substituted by halogen,
(d) a bicyclo cycloalkyl ring wherein each ring is independently a five to six membered cycloalkyl ring,
(e) a tricyclo cycloalkyl ring wherein each ring is independently a five to six membered cycloalkyl ring,
(f) (C₆-C₁₀)-aryl optionally substituted with one to three substituents selected from the group consisting of halogen, (C₁-C₅)-alkoxy and (C₁-C₅)-alkyl optionally substituted by halogen,
(g) —NR₁₁R₁₂, and
(h) a four to eight membered saturated or unsaturated heterocyclic ring which contains one to four heteroatoms selected from the group consisting of nitrogen, oxygen and sulfur wherein said heterocyclic ring contains at least two carbon atoms wherein said heterocyclic ring is optionally substituted with one to four substituents selected from the group consisting of
(h1) nitro,
(h2) NR₉C(═O)R₁₀,
(h3) oxo,
(h4) (C₁-C₅)-alkyl optionally substituted with halogen,
(h5) C(═O)R₁₅,
(h6) C(═O)OR₁₅,
(h7) C(═O)NR₁₃R₁₄,
(h8) (C₆-C₁₀)-aryl optionally substituted with halogen, and
(h9) (C₃-C₈)-cycloalkyl ring;
R₁₁, R₁₂, R₁₃, R₁₄, R₁₇ and R₁₈ are independently selected from the group consisting of:
(a) hydrogen,
(b) (C₁-C₅)-alkyl,
(c) (C₃-C₈)-cycloalkyl,
(d) (C₆-C₁₀)-aryl, and
(e) (C₆-C₁₀)-aryl-(C₁-C₅)-alkyl;
R¹⁵ is hydrogen or (C₁-C₅)-alkyl;
or a purified stereoisomer or stereoisomer mixture of said compound, or salt of said compound, stereoisomer or stereoisomer mixture.

2. The compound of claim 1, wherein
n is 0, 1 or 2;
R is selected from the group consisting of.
(a) (C₁-C₆)-alkyl optionally substituted by (C₃-C₈)-cycloalkyl,
(b) (C₁-C₅)-alkenyl,
(c) (C₁-C₅)-alkynyl, and
(d) (C₆-C₁₀)-aryl which is optionally substituted with one to three substituents selected from the group consisting of:
(d1) halogen,
(d2) nitro,
(d3) (C₁-C₅)-alkyl optionally substituted with halogen,
(d4) (C₁-C₅)-alkenyl optionally substituted with (C₁-C₅)-alkyl,
(d5) (C₁-C₅)-alkynyl optionally substituted with (C₁-C₅)-alkyl,
(d6) (C₁-C₅)-alkoxy,
(d7) NR₉C(═O)R₁₀,
(d8) NR₉S(═O)ₙ—R₁₀,
(d9) NR₁₁R₁₂,
(d10) C(═O)NR₁₃R₁₄,
(d11) (C₆-C₁₀)-aryl optionally substituted with one to three substituents selected from the group consisting of
(d11a) halogen,
(d11b) (C₁-C₅)-alkyl, and
(d11c) (C₁-C₅)-alkoxy,
(d12) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atom, and the other ring is a saturated or unsaturated three to eight membered cycloalkyl ring,
(d13) OH, and
(d14) CN;
R₁ and R₂ are independently selected from the group consisting of:
(a) hydrogen, and
(b) (C₁-C₅)-alkyl optionally substituted with halogen or hydroxy;
R₃, R_{3′}, R₄ and R_{4′} are independently selected from the group consisting of:
(a) hydrogen, and
(b) (C₁-C₅) alkyl;
R₃ and R₄ together form a four to eight membered saturated or unsaturated carbocyclic ring, or
R₄ and R₄ together form a (C₃-C₈)-cycloalkyl ring;
R₅ and R₆ are independently selected from the group consisting of hydrogen and (C₁-C₅)-alkyl;
R₉ is hydrogen or (C₁-C₅)-alkyl;
R₁₀ is selected from the group consisting of:
(a) (C₁-C₅)-alkyl optionally substituted with (C₃-C₈)-carbocyclic ring or (C₆-C₁₀)-aryl optionally substituted with halogen,
(b) (C₃-C₈)-cycloalkyl optionally substituted with one to three substituents selected from the group consisting of halogen, (C₁-C₅)-alkoxy or (C₁-C₅)-alkyl optionally substituted by halogen,
(c) (C₆-C₁₀)-aryl optionally substituted with one to three substituents selected from the group consisting of halogen, (C₁-C₅)-alkoxy and (C₁-C₅)-alkyl optionally substituted by halogen, and
(d) —NR₁₁R₁₂;
R₁₁, R₁₂, R₁₃, R₁₄, R₁₇ and R₁₈ are independently selected from the group consisting of:
(a) hydrogen,
(b) (C₁-C₅)-alkyl,
(c) (C₃-C₈)-cycloalkyl,
(d) (C₆-C₁₀)-aryl, and
(e) (C₆-C₁₀)-aryl-(C₁-C₅)-alkyl

3. The compound of claim 1 wherein
n is 0, 1 or 2;
R is selected from the group consisting of:
(a) (C₆-C₁₀)-aryl which is optionally substituted with one to three substituents selected from the group consisting of:
(a1) halogen,
(a2) nitro,
(a3) (C₁-C₅)-alkyl optionally substituted with halogen,
(a4) (C₁-C₅)-alkenyl optionally substituted with (C₁-C₅)-alkyl,
(a5) (C₁-C₅)-alkynyl optionally substituted with (C₁-C₅)-alkyl,
(a6) (C₁-C₅)-alkoxy,
(a7) NR₉C(═O)R₁₀,
(a8) NR₉S(═O)ₙ—R₁₀,
(a9) NR₁₁R₁₂,
(a10) C(═O)NR₁₃R₁₄,
(a11) (C₆-C₁₀)-aryl optionally substituted with one to three substituents selected from the group consisting of:
(a11a) halogen,
(a11b) (C₁-C₅)-alkyl,
(a11c) (C₁-C₅)-alkoxy,
(a12) a fused bicyclo ring wherein one ring is a four to eight membered saturated or unsaturated heterocyclic ring which contains one to four heteroatoms selected from the group consisting of nitrogen, oxygen and sulfur, wherein said heterocyclic ring contains at least one carbon atom, and the other ring is a saturated or unsaturated three to eight membered cycloalkyl ring;
(a13) OH, and
(a14) CN;
R₁ and R₂ are independently selected from the group consisting of:
(a) hydrogen, and
(b) (C₁-C₅)-alkyl optionally substituted with halogen or hydroxy;
R₃, R_{3′}, R₄ and R_{4′} are independently selected from the group consisting of:
(a) hydrogen, and
(b) (C₁-C₅) alkyl;
R₅ and R₆ are independently selected from the group consisting of hydrogen and methyl,
R₉ is hydrogen or (C₁-C₅)-alkyl;
R₁₀ is selected from the group consisting of:
(a) (C₁-C₅)-alkyl optionally substituted with (C₃-C₈)-carbocyclic ring or (C₆-C₁₀)-aryl optionally substituted with halogen,
(b) (C₃-C₅)-cycloalkyl optionally substituted with one to three substituents selected from the group consisting of halogen, (C₁-C₅)-alkoxy or (C₁-C₅)-alkyl optionally substituted by halogen,
(c) (C₆-C₁₀)-aryl optionally substituted with one to three substituents selected from the group consisting of halogen, (C₁-C₅)-alkoxy and (C₁-C₅)-alkyl optionally substituted by halogen, and
(d) —NR₁₁R₁₂;
R₁, R₁₂, R₁₃, R₁₄, R₁₇ and R₁₈ are independently selected from the group consisting of:
(a) hydrogen,
(b) (C₁-C₅)-alkyl, and
(c) (C₃-C₈)-cycloalkyl.

4. A pharmaceutical composition for the treating or preventing a disease and/or behavior involving the 5-HT_{2C} receptor which comprises a therapeutically effective amount of a compound of claim 1 and one or more pharmaceutically acceptable ingredients.

5. The pharmaceutical composition of claim 4 which further comprises an additional pharmaceutical agent other than a compound of claim 1 for the treatment or prevention a disease and/or behavior involving the 5-HT_{2C} receptor.

6. The pharmaceutical composition of claim 5 wherein said additional agent is an appetite suppressant selected from the group consisting of benzphetamine, diethylpropion, mazindol, phendimetrazine and phentermine.

7. The pharmaceutical composition of claim 5 wherein said additional agent is an agent for treating obesity related disorders selected from the group consisting of insulin-dependent diabetes, non-insulin dependent diabetes, abnormal feeding behavior, eating disorders and premenstrual tension.

8. The pharmaceutical composition of claim 5 wherein said agent for treating obesity related disorders is selected from the group consisting of insulin, tolbutamide, chlorpropamide, tolazamide, acetohexamide, glycburide, glipizide, gliclazide, tricyclic monoamine oxidase (MAO) inhibitors and serotonin reuptake inhibitors.

9. A method of treating or preventing a disease and/or behavior involving the 5-HT_{2C} receptor which comprises administering a therapeutically effective amount of a compound of claim 1 or the composition of claim 4.

10. The method of claim 9 wherein said disease and/or behavior involving the 5-HT_{2C} receptor is selected from the group consisting of obesity, obesity related disorders, abnormal feeding behavior, eating disorders, and premenstrual tension.

11. The method of claim 10 wherein said disease and/or behavior involving the 5-HT_{2C} receptor is obesity.

12. The method of claim 10 wherein said eating disorders are bulimia or anorexia nervosa

13. A method of treating or preventing a disease correlated to obesity selected from the group consisting of Type II diabetes (NIDDM), hypertension, hyperlipidemia, myocardial infarction and dermatological disease which comprises administering a therapeutically effective amount of a compound of claim 1 or the composition of claim 4.
